(19) United States
(12) Patent Application Publication
(10) Pub. No.: US 2025/0084062 A1
(43) Pub. Date: Mar. 13, 2025

(54) SUBSTITUTED QUINOLINE DERIVATIVE

(71) Applicant: ASTELLAS PHARMA INC., Tokyo (JP)

(72) Inventors: Ryuichi SEKIOKA, Tokyo (JP); Eriko HONJO, Tokyo (JP); Kei OHNUKI, Tokyo (JP); Yohei KOGANEMARU, Tokyo (JP); Takuya WASHIO, Tokyo (JP); Ayaka MORIKAWA, Tokyo (JP); Kenji NEGORO, Tokyo (JP); Shota SATO, Tokyo (JP); Sadanori MIYOSHI, Tokyo (JP); Takao SUZUKI, Shanghai (CN)

(73) Assignee: ASTELLAS PHARMA INC., Tokyo (JP)

(21) Appl. No.: 18/723,117

(22) PCT Filed: Dec. 26, 2022
(86) PCT No.: PCT/JP2022/047962

§ 371 Date: Jun. 21, 2024

(30) Foreign Application Priority Data
Dec. 27, 2021 (WO) PCT/CN2021/141500

(51) Int. Cl. C07D 401/12; A61K 31/40; A61K 31/4375; A61K 31/4545; A61K 31/4709; A61K 31/496; A61K 31/5377; A61P 21/00; C07D 207/48; C07D 401/14; C07D 471/04; C07D 487/10

(52) U.S. Cl. CPC C07D 401/12; A61K 31/40; A61K 31/4375; A61K 31/4545; A61K 31/4709; A61K 31/496; A61K 31/5377; A61P 21/00; C07D 207/48; C07D 401/14; C07D 471/04; C07D 487/10

(57) ABSTRACT

The present inventors have studied and found that the substituted quinoline derivatives have acetylcholine receptor clustering-inducing action and can be useful as an active ingredient in the pharmaceutical compositions for preventing and/or treating neuromuscular diseases. The substituted quinoline derivative of the present invention may be used as an agent for preventing and/or treating neuromuscular diseases.

### TECHNICAL FIELD

The present invention relates to substituted quinoline derivatives that induce acetylcholine receptor clustering and may be useful as an active ingredient of a pharmaceutical composition, for example, a pharmaceutical composition for preventing and/or treating neuromuscular diseases.

### BACKGROUND ART

The neuromuscular junction (NMJ) is a chemical synapse formed between a motor nerve axon terminal and skeletal muscle and is essential for controlling skeletal muscle contraction via an endogenous neurotransmitter, acetylcholine. Acetylcholine receptors are highly clustered in the postsynaptic region formed on the skeletal muscle end plate, and such clustering plays an important role in functional NMJ formation. It is known that failure or breakdown of acetylcholine receptor higher clustering is associated with decreased skeletal muscle contractile function in neuromuscular diseases, including congenital myasthenia caused by mutations or defects in NMJ-related genes and myasthenia gravis caused by autoantibodies against NMJ constituent proteins (NPL 1).

The acetylcholine receptor expressed in skeletal muscle is a pentameric ligand-gated ion channel formed by the assembly of two subunits of alpha 1 and one subunit each of beta 1, delta, and epsilon. In acetylcholine receptors of the non-innervated muscles, such as fetuses in the developmental stage, the pentamer is similar to innervated muscle except a gamma subunit is substituted for the epsilon subunit. It is known that the postsynaptic compartment is formed by significant clustering of the pentameric acetylcholine receptor, and that the acetylcholine receptors exist in the postsynaptic region at a density approximately 1000 times higher than the surroundings (NPL 2).

It has been reported that activation of the receptor tyrosine kinase MuSK on the skeletal muscle end plate, via low-density lipoprotein receptor-related protein 4 (Lrp4), by glycoprotein Agrin secreted from the motor nerve axon terminal is essential for significant clustering and maintenance of acetylcholine receptors (NPL 3). It is believed that, during MuSK activation, a MuSK, in which a dimer of Agrin combines with a dimer of Lrp4 to form a tetramer, approaches another MuSK in the same state and causes autophosphorylation in the cytoplasmic tyrosine kinase domain of MuSK. The activation of MuSK causes changes in the localization and function of various proteins, including Rapsyn, which acts as a scaffold protein, leading to acetylcholine receptor clustering. It has also been reported that DOK7, identified as one of the causative genes for congenital myasthenia, promotes phosphorylation of MuSK within the cell and induces muscle-autonomous activation of acetylcholine receptor clustering before motor innervation at ontogenesis (NPL 4). In this way, various mechanisms of NMJ formation and maintenance have been elucidated in recent years, but many of the details are still unknown.

Artificial induction of significant acetylcholine receptor clustering has been investigated as a study of therapeutic methods for diseases targeting NMJs, and improvement of pathological conditions in multiple disease animal model have been reported (NPL 5). In addition to neuromuscular diseases models such as congenital myasthenia and myasthenia gravis, models of neurodegenerative disease such as amyotrophic lateral sclerosis and myelopathic muscular atrophy, as well as sarcopenia characterized by age-related skeletal muscle mass decrease, therapeutic effect of induction of acetylcholine receptor clustering have been reported (NPL 6). It has shown that induction of acetylcholine receptor clustering can provide treatment for various diseases associated with decreased NMJ function and decreased skeletal muscle contraction function.

Techniques for inducing acetylcholine receptor clustering have been reported: introducing the DOK7 gene with adeno-associated viruses to induce MuSK activation from inside of the cell (NPL 7, NPL 8, and NPL 9), antibody-induced dimerization of MuSK from outside of the cell (PTL 1, NPL 10, and NPL 11), decomposing Agrin into small molecules by genetic modification (PTL 2, NPL 12, NPL 13 and NPL 14), and the like.

NPL 15 have reported that salbutamol, as shown in the following formula, has an inducing action on acetylcholine receptor clustering.

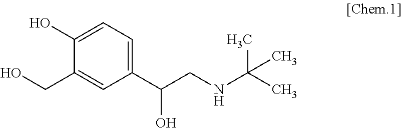 [SMILES: CC(C)(C)NCC(O)c1ccc(O)c(CO)c1]

NPL 16 discloses that the combination of the compound shown in the following formula and Agrin promotes an inducing action on acetylcholine receptor clustering.

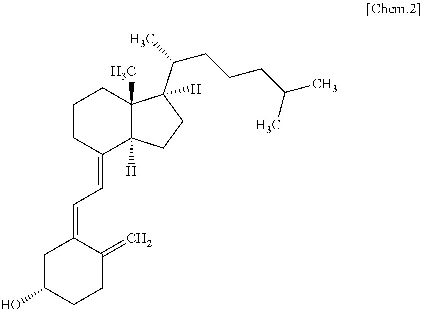 [SMILES: C=C1CC[C@H](O)C/C1=C/C=C1\CCC[C@]2(C)[C@@H]([C@H](C)CCCC(C)C)CC[C@@H]12]

### CITATION LIST

### Patent Literature

PTL 1: WO2013/074636
PTL 2: WO2011/026615

### Non-Patent Literature

NPL 1: Annu. Rev. Physiol., 80, 159-188 (2018)
NPL 2: J. Cell Biol., 69, 144-158 (1976)
NPL 3: Expert Opin. Ther. Targets, 21:10, 949-958 (2017)
NPL 4: Sci. Signal., 2, ra7 (2009)
NPL 5: Front. Mol. Neurosci., 13, 610964 (2020)
NPL 6: iScience, 23, 101385 (2020)
NPL 7: Science, 345, 1505-1508 (2014)
NPL 8: EMBO Mol. Med., 9, 880-889 (2017)
NPL 9: iScience, 23, 101385 (2020)
NPL 10: eLife, 7, 34375 (2018)
NPL 11: Nature, 595, 404-408 (2021)
NPL 12: PLOS One, e88739 (2014)
NPL 13: Muscle Nerve, 57, 814-820 (2018)
NPL 14: Front. Cell. Neurosci., 12, 17 (2018)
NPL 15: Journal of Neuromuscular Diseases, 5: pp 231-240 (2018)
NPL 16: Biochemical and Biophysical Research Communications, 525: pp 80-86 (2020)

### SUMMARY OF INVENTION

### Technical Problem

Provided are pharmaceutical compositions, in particular compounds, that have an inducing action on acetylcholine receptor clustering and are useful as active ingredients in pharmaceutical compositions for preventing and/or treating neuromuscular diseases.

### Solution to Problem

The present inventors have studied and found that substituted quinoline derivatives have inducing action on acetylcholine receptor clustering and can be useful as an active ingredient in the pharmaceutical compositions for preventing and/or treating neuromuscular diseases.

The present invention relates to a compound of formula (I) or a salt thereof and a pharmaceutical composition comprising a compound of formula (I) or a salt thereof and one or more excipients.

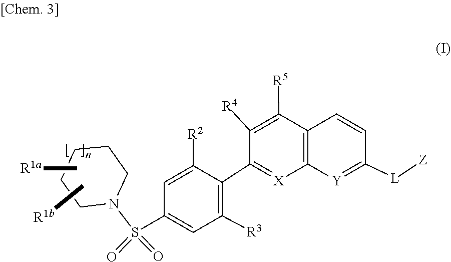

wherein,
R^{1a} and R^{1b} each are the same or different, and are H, optionally substituted C₁₋₆ alkyl, halogen, hydroxy, or —O-(optionally substituted C₁₋₆ alkyl), and when R^{1a} and R^{1b} are attached to the same carbon atom, R^{1a} and R^{1b} may be linked to each other to form C₃₋₈ cycloalkyl group together with the carbon atom to which R^{1a} and R^{1b} are attached,
R² is H, optionally substituted C₁₋₆ alkyl, halogen, cyano, or —O-(optionally substituted C₁₋₆ alkyl),
R³ is H or halogen,
R⁴ is H, methyl, or halogen,
R⁵ is methyl, ethyl, or fluoromethyl,
X is N or CR^{X} and Y is N or CR^{Y},
R^{X} is H or halogen,
R^{Y} is H or halogen,
L is a bond, C₁₋₆ alkylene, —O—(C₁₋₆ alkylene), C₂₋₆ alkenylene, C₃₋₈ Cycloalkylene, or C₄₋₈ cycloalkenylene,
Z is —COOH or —CONR^{Z1}R^{Z2}, or
L and Z may together form optionally substituted C₁₋₆ alkyl, —O-(optionally substituted C₁₋₆ alkyl), or optionally substituted 3- to 8-membered heterocyclic ring group comprising 1 to 2 nitrogen atoms,
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₁₋₆ alkyl or C₃₋₈ cycloalkyl,
R^{Z2} is H or C₁₋₆ alkyl, or
R^{Z1} and R⁷² may be linked to each other to form optionally substituted 3- to 8-membered heterocyclic ring group comprising 1 to 2 nitrogen atoms together with the nitrogen atom to which R^{Z1} and R^{Z2} are attached, and,
n is 0 or 1.

The present invention also relates to a compound of formula (I) or a salt thereof and a pharmaceutical composition comprising a compound of formula (I) or a salt thereof and one or more excipients.

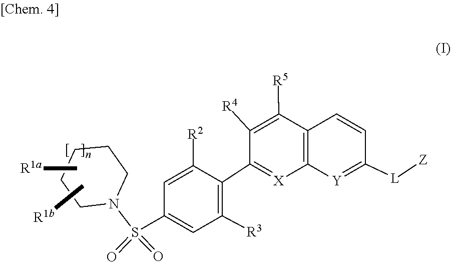

wherein,
R^{1a} and R^{1b} each are the same or different, and are H, optionally substituted C₁₋₆ alkyl, halogen, hydroxy, or —O-(optionally substituted C₁₋₆ alkyl), and when R^{1a} and R^{1b} are attached to the same carbon atom, R^{1a} and R^{1b} may be linked to each other to form C₃₋₈ cycloalkyl group together with the carbon atom to which R^{1a} and R^{1b} are attached,
R² is H, optionally substituted C₁₋₆ alkyl, halogen, cyano, or —O-(optionally substituted C₁₋₆ alkyl),
R³ is H or halogen,
R⁴ is H, methyl, or halogen,
R⁵ is methyl, ethyl, or fluoromethyl,
X is N or CR^{X}, Y is N or CR^{Y}, provided that X and Y are not CR^{X} and CR^{Y} at the same time,
R^{X} is H or halogen,
R^{Y} is H or halogen,
L is a bond, C₁₋₆ alkylene, —O—(C₁₋₆ alkylene), C₂₋₆ alkenylene, C₃₋₈ cycloalkylene, or C₄₋₈ cycloalkenylene,
Z is —COOH or —CONR^{Z1}R^{Z2}, or
L and Z may together form optionally substituted C₁₋₆ alkyl, —O-(optionally substituted C₁₋₆ alkyl), or optionally substituted 3- to 8-membered heterocyclic ring group comprising 1 to 2 nitrogen atoms,
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₁₋₆ alkyl or C₃₋₈ cycloalkyl,
R^{Z2} is H or C₁₋₆ alkyl, or
R^{Z1} and R^{Z2} may be linked to each other to form optionally substituted 3- to 8-membered heterocyclic ring group comprising 1 to 2 nitrogen atoms together with the nitrogen atom to which R^{Z1} and R^{Z2} are attached, and
n is 0 or 1.
   Unless otherwise indicated herein, when the symbol in one chemical formula is also used in another chemical formula, the same symbol indicates the same meaning.

Further, the present invention relates to a pharmaceutical composition for preventing and/or treating neuromuscular diseases that contains a compound of formula (I) or a salt thereof and pharmaceutically acceptable excipients. The pharmaceutical composition contains an agent for preventing and/or treating neuromuscular diseases that contain(s) a compound of formula (I) or a salt thereof.

The present invention relates to a compound of formula (I) or a salt thereof, which is an acetylcholine receptor clustering inducing agent, a compound of formula (I) or a salt thereof for use as an acetylcholine receptor clustering inducing agent, an acetylcholine receptor clustering inducing agent comprising a compound of formula (I) or a salt thereof, use of a compound of formula (I) or a salt thereof for the manufacture of a pharmaceutical composition for preventing and/or treating neuromuscular diseases, use of a compound of formula (I) or a salt thereof for preventing and/or treating of neuromuscular diseases, a compound of formula (I) or a salt thereof for use in preventing and/or treating of neuromuscular diseases, and a method for preventing and/or treating neuromuscular diseases, comprising administering an effective amount of a compound of formula (I) or a salt thereof to a subject.

The term “subject” refers to a human or animal, and in one embodiment, a human.

### Advantageous Effects of Invention

The compound of formula (I) or a salt thereof has an acetylcholine receptor clustering-inducing action and can be useful as an agent for preventing and/or treating neuromuscular diseases.

### BRIEF DESCRIPTION OF DRAWINGS

FIG. 1 shows the results of evaluating the effect of the compound of Example 2 on suppressing the decrease in grip strength in a MuSK-type myasthenia gravis animal model. The vertical axis shows the muscle strength (kg) of the extremities of the mouse measured by the grip strength measuring device, and is shown the mean plus/minus standard error of mean.

FIG. 2 shows the results of evaluating the effect of the compound of Example 18 and Example 19 on suppressing the decrease in grip strength in a MuSK-type myasthenia gravis animal model. The vertical axis shows the muscle strength (kg) of the extremities of the mouse measured by the grip strength measuring device, and is shown the mean plus/minus standard error of mean.

### DESCRIPTION OF EMBODIMENTS

Hereinafter, the present invention will be described in detail.

In the present specification, the following terms have the meanings shown below unless otherwise specified. The definitions below are intended to clarify the terms defined and are not intended to limit the definitions. If the term used herein is not specifically defined, it will have a meaning generally accepted by a person skilled in the art.

In the present invention, “alkyl” is a linear or branched alkyl. Accordingly, “C₁₋₆ alkyl” is a linear or branched alkyl having 1 to 6 carbon atoms (Hereinafter, the number of carbon atoms is described in the same manner.), for example, methyl, ethyl, n-propyl, isopropyl, n-butyl, isobutyl, sec-butyl, tert-butyl, n-pentyl, or n-hexyl. “C₁₋₆ alkyl” is C₁₋₃ alkyl in one embodiment, methyl, ethyl or isopropyl in another embodiment, methyl or ethyl in another embodiment, and methyl in yet another embodiment.

“C₁₋₃ alkyl” is, for example, methyl, ethyl, n-propyl or isopropyl. “C₁₋₃ alkyl” is methyl or ethyl in one embodiment, and methyl in another embodiment.

“Alkylene” is a divalent group formed by removing a hydrogen atom from above “Alkyl”. Accordingly, “C₁₋₆ alkylene” is, for example, methylene, ethylene, trimethylene, tetramethylene, pentamethylene, hexamethylene, propylene, methylmethylene, ethylethylene, 1,2-dimethylethylene, or 1,1,2,2-tetramethylethylene. “C₁₋₆ alkylene” is methylene, ethylene or propylene in one embodiment, methylene in another embodiment, and ethylene in yet another embodiment.

“Cycloalkyl” is a saturated hydrocarbon ring group optionally crosslinked or spirocyclized. “C₃₋₈ cycloalkyl” is, for example, cyclopropyl, cyclobutyl, cyclopentyl, cyclohexyl, cycloheptyl, or cyclooctyl. “C₃₋₈ cycloalkyl” is cyclopropyl in one embodiment.

“Cycloalkylene” is a divalent group formed by removing a hydrogen atom from above “Cycloalkyl”. Accordingly, “C₃₋₈ cycloalkylene” is, for example, cyclopropanediyl, cyclobutanediyl, cyclopentanediyl, cyclohexanediyl, cycloheptanediyl, or cyclooctanediyl. “C₃₋₈ cycloalkylene” is cyclohexanediyl in one embodiment.

“Alkenyl” is a linear or branched alkyl having one double bond in the “Alkyl”. Accordingly, “C₂₋₆ alkenyl” is, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, or 5-hexenyl. “C₂₋₆ alkenyl” is ethenyl in one embodiment.

“Alkenylene” is a divalent group formed by removing a hydrogen atom from above “Alkenyl”. Accordingly, “C₂₋₆ alkenylene” is, for example, ethenylene, 1-propenylene, 2-propenylene, 2-methyl-1-propenylene, 1-butenylene, 2-butenylene, 3-butenylene, 3-methyl-2-butenylene, 1-pentenylene, 2-pentenylene, 3-pentenylene, 4-pentenylene, 4-methyl-3-pentenylene, 1-hexenylene, 3-hexenylene, or 5-hexenylene. “C₂₋₆ alkenylene” is ethenylene in one embodiment.

“Cycloalkenyl” is an unsaturated hydrocarbon ring group and has one double bond in the ring. Accordingly, “C₄₋₈ cycloalkenyl” is, for example, cyclobutenyl, cyclopentenyl, cyclohexenyl, cycloheptenyl or cyclooctenyl.

“Cycloalkenylene” is a divalent group formed by removing a hydrogen atom from above “Cycloalkenyl”. Accordingly, “C₄₋₈ cycloalkenylene” is, for example, cyclobutenediyl, cyclopentenediyl, cyclohexenediyl, cycloheptenediyl or cyclooctenediyl. “C₄₋₈ cycloalkenylene” is cyclohexenediyl in one embodiment.

“Heterocyclic ring group” is a 3- to 8-membered heterocyclic ring group, having 1 to 4 heteroatoms selected from the group consisting of nitrogen atom, oxygen atom, and sulfur atom as the ring-constituting atom optionally crosslinked with C₁₋₆ alkylene, optionally forming additional 3 to 6-membered spiro-ring which may have a nitrogen atom as the ring-constituting atom, and optionally having oxidized sulfur atom as the ring-constituting atom, and optionally have a double bond in the ring, and specifically, for example, azepanyl, diazepanyl, oxazepanyl, thiazepanyl, aziridinyl, azctidinyl, pyrrolidinyl, imidazolidinyl, piperidinyl, pyrazolydinyl, pipcrazinyl, azepanyl, azocanyl, thiomorpholinyl, thiazolidinyl, isothiazolidinyl, oxazolidinyl, morpholinyl, tetrahydrothiopyranyl, oxathiolanyl, oxiranyl, oxctanyl, dioxolanyl, tetrahydrofuranyl, tetrahydropyranyl, tetrahydropyridyl 1,4-dioxanyl or 2,6-diazaspiro[3.3]heptanyl.

Among the “Heterocyclic ring group”, “3- to 8-membered heterocyclic ring group comprising 1 to 2 nitrogen atoms” is 3- to 8-membered heterocyclic ring group, having 1 to 2 nitrogen atoms which may also have a double bond in the ring, for example, aziridinyl, azctidinyl, pyrrolidinyl, piperidinyl, piperazinyl, azepanyl, azocanyl, tetrahydropyridyl or 2,6-diazaspiro[3.3]heptanyl. “3- to 8-membered heterocyclic ring group comprising 1 to 2 nitrogen atoms” is azetidinyl, pyrrolidinyl, piperidinyl, piperazinyl, tetrahydropyridyl or 2,6-diazaspiro[3.3]heptanyl in one embodiment, piperidinyl, piperazinyl or tetrahydropyridyl in another embodiment, azetidinyl or pyrrolidinyl in another embodiment, azetidinyl or 2,6-diazaspiro[3.3]heptanyl in another embodiment, azetidinyl in another embodiment, 2,6-diazaspiro[3.3]heptanyl in yet another embodiment.

“Halogen” is fluoro, chloro, bromo or iodo, and is fluoro, chloro, or bromo in one embodiment, fluoro or chloro in one embodiment, fluoro in another embodiment, and chloro in yet another embodiment.

In the present invention, “optionally substituted” means unsubstituted or substituted with one or more substituents and is optionally substituted with 1 to 5 substituents in one embodiment, and is optionally substituted with 1 to 2 substituents in one embodiment. The substitution may be performed at any position in the group where hydrogen is normally present.

The substituents in the “optionally substituted C₁₋₆ alkyl” in Ria, R^{1b} and R² are hydroxy or —O—(C₁₋₆ alkyl) in one embodiment, hydroxy or methoxy in another embodiment, hydroxy in yet another embodiment.

The substituents of the “optionally substituted C₁₋₆ alkyl” and “—O-(optionally substituted C₁₋₆ alkyl)”, each of which is formed by L and Z together, are hydroxy, —NH₂, —NH(C₁₋₆ alkyl) or —N(C₁₋₆ alkyl)₂ in one embodiment, —NH₂, —NH(C₁₋₆ alkyl) or —N(C₁₋₆ alkyl)₂ in another embodiment, hydroxy, —NH₂ and —NH(C₁₋₆ alkyl) in another embodiment, hydroxy, —NH₂ and —NH-methyl in another embodiment, hydroxy or —NH₂ in another embodiment, —NH₂ or —NH(C₁₋₆ alkyl) in another embodiment, —NH₂ or —NH-methyl in yet another embodiment.

The substituents in the “optionally substituted C₁₋₆ alkyl” in R⁷¹ are —NH₂, —NH(C₁₋₆ alkyl), —N(C₁₋₆ alkyl)₂, —COOH, or heterocyclic ring group optionally substituted with hydroxy in one embodiment, —NH₂, —NH-methyl, —N(methyl)₂, —COOH, hydroxyazetidinyl or morpholinyl in another embodiment.

The substituents of the “optionally substituted 3- to 8-membered heterocyclic ring group comprising 1 to 2 nitrogen atoms” are C₁₋₆ alkyl, —NH₂, —COOH or oxo in one embodiment, methyl, —NH₂, —COOH or oxo in another embodiment, C₁₋₆ alkyl or oxo in another embodiment, methyl or oxo in another embodiment, C₁₋₆ alkyl, —NH₂ or —COOH in another embodiment, methyl, —NH₂ or —COOH in another embodiment, C₁₋₆ alkyl in another embodiment, methyl in yet another embodiment.

In the present invention, “acetylcholine receptor clustering inducing agent” means a compound which induce an acetylcholine receptor clustering. “Acetylcholine receptor clustering inducing agent” in one embodiment, is a compound having a Max value of 30% or more at the compound concentration of 30 μM or less in the method of Test Example 1 described herein.

One or more embodiments can be combined with another embodiment, even if the combination is not specifically described. That is, all embodiments can be freely combined.

In the present invention, “neuromuscular diseases” is a group of diseases, including but not limited to, myasthenia gravis, congenital myasthenia, amyotrophic lateral sclerosis, myelopathic muscular atrophy, peripheral neuropathy, or age-related sarcopenia, and in one embodiment, refers to myasthenia gravis, congenital myasthenia, amyotrophic lateral sclerosis, or myelopathic muscular atrophy, and in another embodiment, refers to myasthenia gravis.

Some embodiments of the present invention of the compound of formula (I), or a salt thereof are shown below.
(1-1) A compound or a salt thereof, wherein R^{1a} and R^{1b} each are the same or different, and are H, optionally substituted C₁₋₆ alkyl, halogen, hydroxy, or —O-(optionally substituted C₁₋₆ alkyl), and when R^{1a} and R^{1b} are attached to the same carbon atom, R^{1a} and R^{1b} may be linked to each other to form C₃₋₈ cycloalkyl group together with the carbon atom to which Ria and R^{1b} are attached.
(1-2) A compound or a salt thereof, wherein Ria and R^{1b} each are the same or different, and are H, methyl, hydroxymethyl, fluoro, hydroxy, or methoxy, and when R^{1a} and R^{1b} are attached to the same carbon atom, R^{1a} and R^{1b} may be linked to each other to form a cyclopropyl group together with the carbon atom to which R^{1a} and R^{1b} are attached.
(1-3) A compound or a salt thereof, wherein R^{1a} and R^{1b} each are the same or different and, are H or fluoro.
(2-1) A compound or a salt thereof, wherein R² is H, optionally substituted C₁₋₆ alkyl, halogen, cyano, or —O-(optionally substituted C₁₋₆ alkyl).
(2-2) A compound or a salt thereof, wherein R² is H, methyl, hydroxymethyl, methoxymethyl, fluoro, cyano, or methoxy.
(2-3) A compound or a salt thereof, wherein R² is H or fluoro.
(2-4) A compound or a salt thereof, wherein R² is fluoro.
(3-1) A compound or a salt thereof, wherein R³ is H or halogen.
(3-2) A compound or a salt thereof, wherein R³ is H or fluoro.
(3-3) A compound or a salt thereof, wherein R³ is fluoro.
(4-1) A compound or a salt thereof, wherein R⁴ is H, methyl, or halogen.
(4-2) A compound or a salt thereof, wherein R⁴ is H, or halogen.
(4-3) A compound or a salt thereof, wherein R⁴ is H or fluoro.
(5-1) A compound or a salt thereof, wherein R⁵ is methyl, ethyl, or fluoromethyl.
(5-2) A compound or a salt thereof, wherein R⁵ is methyl.
(6-1) A compound or a salt thereof, wherein X is N or CR^{X} and Y is N or CR^{Y}, provided that X and Y are not CR^{X} and CR^{Y} at the same time.
(6-2) A compound or a salt thereof, wherein X is N and Y is CR^{Y}.
(6-3) A compound or a salt thereof, wherein X is CR^{X} and Y is N.
(6-4) A compound or a salt thereof, wherein X is N and Y is N.
(6-5) A compound or a salt thereof, wherein X is N or CR^{X} and Y is N or CR^{Y}.
(6-6) A compound or a salt thereof, wherein X is CR^{X} and Y is CR^{Y}.
(7-1) A compound or a salt thereof, wherein, R^{X} is H or halogen.
(7-2) A compound or a salt thereof, wherein, R^{X} is H or fluoro.
(7-3) A compound or a salt thereof, wherein, R^{X} is H.
(8-1) A compound or a salt thereof, wherein, R^{Y} is H or halogen.
(8-2) A compound or a salt thereof, wherein, R^{Y} is H or fluoro.
(8-3) A compound or a salt thereof, wherein, R^{Y} is H.
(9-1) A compound or a salt thereof, wherein
L is a bond, C₁₋₆ alkylene, —O—(C₁₋₆ alkylene), C₂₋₆ alkenylene, C₃₋₈ cycloalkylene, or C₄₋₈ cycloalkenylene,
Z is —COOH or —CONR^{Z1}R^{Z2}, or
L and Z may together form optionally substituted C₁₋₆ alkyl, —O-(optionally substituted C₁₋₆ alkyl), or optionally substituted 3- to 8-membered heterocyclic ring group comprising 1 to 2 nitrogen atoms.
(9-2) A compound or a salt thereof, wherein
L is a bond, C₁₋₆ alkylene, —O—(C₁₋₆ alkylene), C₂₋₆ alkenylene, C₃₋₈ Cycloalkylene, or C₄₋₈ cycloalkenylene,
Z is —COOH or —CONR^{Z1}R^{Z2}, or
L and Z may together form C₁₋₆ alkyl optionally substituted with the 1 to 2 substituents selected from the group consisting of hydroxy and —NHR^{LZ}, —O—(C₁₋₆ alkyl optionally substituted with 1 to 2 substituents selected from the group consisting of hydroxy and —NHR^{LZ}), or 3- to 8-membered heterocyclic ring group comprising 1 to 2 nitrogen atoms optionally substituted with the 1 to 2 substituents selected from the group consisting of C₁₋₆ alkyl and oxo, and
R^{LZ} is H or C₁₋₆ alkyl.
(9-3) A compound or a salt thereof, wherein L is a bond, methylene, ethylene, —OCH₂—, ethenylene, cyclohexanediyl, or cyclohexenediyl,
Z is —COOH or —CONR^{Z1}R^{Z2}, or
L and Z may together form C₁₋₃ alkyl optionally substituted with a substituent selected from the group consisting of —NH₂ and —NH-methyl, —O—(C₁₋₃ alkyl optionally substituted with 1 to 2 substituents selected from the group consisting of hydroxy and —NH 2), or 3- to 8-membered heterocyclic ring group comprising 1 to 2 nitrogen atoms optionally substituted with a substituent selected from the group consisting of methyl and oxo.
(9-4) A compound or a salt thereof, wherein
L is a bond,
Z is —COOH or —CONR^{Z1}R^{Z2}.
(9-5) A compound or a salt thereof, wherein
L is a bond,
Z is —COOH.
(9-6) A compound or a salt thereof, wherein
L is a bond,
Z is —CONR^{Z1}R^{Z2}.
(10-1) A compound or a salt thereof, wherein
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₁₋₆ alkyl or C₃₋₈ cycloalkyl,
R^{Z2} is H or C₁₋₆ alkyl, or
R^{Z1} and R⁷² may be linked to each other to form optionally substituted 3- to 8-membered heterocyclic ring group comprising 1 to 2 nitrogen atoms together with the nitrogen atom to which R^{Z1} and R^{Z2} are attached.
(10-2) A compound or a salt thereof, wherein
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₃₋₈ cycloalkyl,
R^{Z2} is H or C₁₋₆ alkyl, or
R^{Z1} and R^{Z2} may be linked to each other to form optionally substituted 3- to 8-membered heterocyclic ring group comprising 1 to 2 nitrogen atoms together with the nitrogen atom to which R^{Z1} and R^{Z2} are attached.
(10-3) A compound or a salt thereof, wherein
R^{Z1} is C₁₋₃ alkyl optionally substituted with 1 to 2 substituents selected from the group consisting of —NH₂, —NH-methyl, —N(methyl)₂ and morpholinyl, 3- to 8-membered heterocyclic ring group comprising 1 to 2 nitrogen atoms optionally substituted with a methyl, or —SO₂—R^{Z3},
or R^{Z1} is the substituent selected from the group consisting of the following formula (i), formula (ii) and formula (iii),

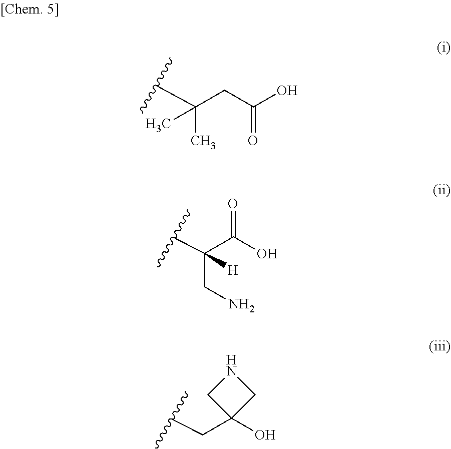

R^{Z3} is cyclopropyl,
R^{Z2} is H or methyl, or
R^{Z1} and R^{Z2} may be linked to each other to form structure(s) of the following formula (iv) or formula (v) together with the nitrogen atom to which R^{Z1} and R⁷² are attached, and

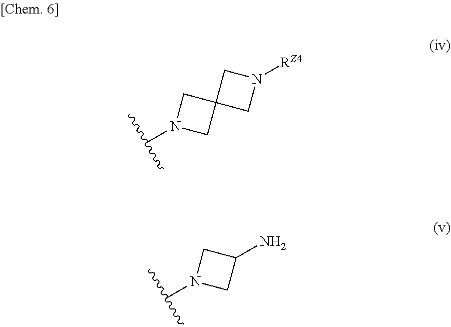

R^{Z4} is H or methyl.
(10-4) A compound or a salt thereof, wherein
R^{Z1} and R^{Z2} are linked to each other to form structure(s) of the following formula (vi) or formula (v) together with the nitrogen atom to which R^{Z1} and R^{Z2} are attached.

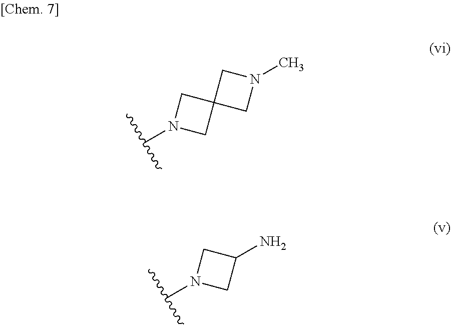

(10-5) A compound or a salt thereof, wherein

R^{Z1} and R^{Z2} are linked to each other to form a structure of the following formula (vi) together with the nitrogen atom to which R^{Z1} and R⁷² are attached.

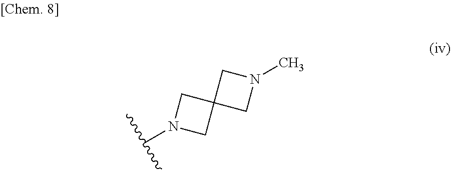 [SMILES: *N1CC2(C1)CN(C)C2]

(10-6) A compound or a salt thereof, wherein
R^{Z1} and R^{Z2} are linked to each other to form a structure of the following formula (v) together with the nitrogen atom to which R^{Z1} and R⁷² are attached.

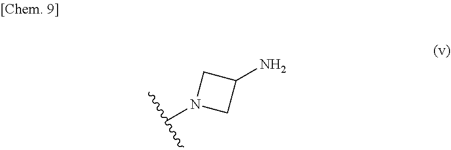 [SMILES: *N1CC(N)C1]

(11-1) A compound or a salt thereof, wherein n is 0 or 1.
(11-2) A compound or a salt thereof, wherein n is 0.
(11-3) A compound or a salt thereof, wherein n is 1.
(12) A compound or a salt thereof which is a combination of two or more embodiments described in (1-1) to (11-3) above that are consistent with each other. Examples of the combination include the followings, though not limited thereto.
(12-1) A compound or a salt thereof which is a combination of the embodiments (1-1), (2-1), (3-1), (4-1), (5-1), (6-5), (7-1), (8-1), (9-1), (10-1), and (11-1) above.
(12-2) A compound or a salt thereof which is a combination of embodiments (1-2), (2-2), (3-2), (4-3), (5-2), (6-5), (7-2), (8-2), (9-3), (10-3), and (11-1) above.
(12-3) A compound or a salt thereof which is a combination of the embodiments (1-1), (2-1), (3-1), (4-1), (5-1), (6-1), (7-1), (8-1), (9-1), (10-1), and (11-1) above.
(12-4) A compound or a salt thereof which is a combination of the embodiments (1-1), (2-1), (3-1), (4-2), (5-2), (6-1), (7-1), (8-1), (9-2), (10-2), and (11-1) above.
(12-5) A compound or a salt thereof which is a combination of embodiments (1-2), (2-2), (3-2), (4-3), (5-2), (6-1), (7-2), (8-2), (9-3), (10-3), and (11-1) above.
(12-6) A compound or a salt thereof which is a combination of embodiments (1-3), (2-4), (3-3), (4-3), (5-2), (6-2), (8-3), (9-4), (10-4), and (11-1) above.

Examples of the specific compound included in the present invention are as follows:
2-{2,6-Difluoro-4-[(3S)-3-fluoropyrrolidine-1-sulfonyl]phenyl}-4-methylquinoline-7-carboxylic acid,
Sodium 2-{2,6-difluoro-4-[(3S)-3-fluoropyrrolidine-1-sulfonyl]phenyl}-4-methylquinoline-7-carboxylate,
{2-[2,6-Difluoro-4-(4-fluoropiperidine-1-sulfonyl)phenyl]-4-methylquinolin-7-yl}(6-methyl-2,6-diazaspiro[3.3]heptan-2-yl) methanone,
(3-Aminoazetidin-1-yl) {2-[2,6-difluoro-4-(4-fluoropiperidine-1-sulfonyl)phenyl]-4-methylquinolin-7-yl}methanone,
2-[2,6-Difluoro-4-(4-fluoropiperidine-1-sulfonyl)phenyl]-3-fluoro-4-methylquinoline-7-carboxylic acid,
2-[2,6-Difluoro-4-(4-fluoropiperidine-1-sulfonyl)phenyl]-4-methylquinoline-7-carboxylic acid,
2-{2,6-Difluoro-4-[(3S)-3-fluoropyrrolidine-1-sulfonyl]phenyl}-3-fluoro-4-methylquinoline-7-carboxylic acid.

Examples of specific compounds included in the present invention include the following compounds in one embodiment.

A compound or a salt thereof selected from the group consisting of
2-{2,6-Difluoro-4-[(3S)-3-fluoropyrrolidine-1-sulfonyl]phenyl}-4-methylquinoline-7-carboxylic acid,
Sodium 2-{2,6-difluoro-4-[(3S)-3-fluoropyrrolidine-1-sulfonyl]phenyl}-4-methylquinoline-7-carboxylate,
{2-[2,6-Difluoro-4-(4-fluoropiperidine-1-sulfonyl)phenyl]-4-methylquinolin-7-yl}(6-methyl-2,6-diazaspiro[3.3]heptan-2-yl) methanone,
(3-Aminoazetidin-1-yl) {2-[2,6-difluoro-4-(4-fluoropiperidine-1-sulfonyl)phenyl]-4-methylquinolin-7-yl}methanone,
2-[2,6-Difluoro-4-(4-fluoropiperidine-1-sulfonyl)phenyl]-3-fluoro-4-methylquinoline-7-carboxylic acid,
2-[2,6-Difluoro-4-(4-fluoropiperidine-1-sulfonyl)phenyl]-4-methylquinoline-7-carboxylic acid,
2-{2,6-Difluoro-4-[(3S)-3-fluoropyrrolidine-1-sulfonyl]phenyl}-3-fluoro-4-methylquinoline-7-carboxylic acid.

With regards to the compound of formula (I), tautomers or geometrical isomers thereof may exist, depending on the types of the substituents. In the present specification, the compound of formula (I), or a salt thereof may be described in one form of isomer, but the present invention includes other isomers, isolated forms of the isomers, or mixtures thereof.

In addition, the compound of formula (I), or a salt thereof may have an asymmetric center or an axial chirality and an enantiomer (optical isomer) thereof due to the asymmetric center or the axial chirality may exist. The compound of formula (I), or a salt thereof includes any of the isolated individual enantiomers, such as (R)-form and (S)-form, and mixtures thereof (including racemic and non-racemic mixtures). In one embodiment, an enantiomer is “stereochemically pure.” “Stereochemically pure” means a degree of purity that can be recognized by a person skilled in the art as being substantially stereochemically pure. In another embodiment, an enantiomer is, for example, a compound having a stereochemical purity of 90% ee (enantiomeric excess) or higher, 95% ee or higher, 98% ee or higher, or 99% ee or higher.

Furthermore, the present invention also includes pharmaceutically acceptable prodrugs of the compound of formula (I). A pharmaceutically acceptable prodrug is a compound having a group that can be converted into an amino group, a hydroxy group, a carboxyl group, or the like by solvolysis or under physiological conditions. Examples of the groups forming prodrugs include those described in Prog. Med., 5, 2157-2161 (1985) or “Pharmaceutical Research and Development” (Hirokawa Publishing Company, 1990), Vol. 7, Molecular Design 163-198.

The salt of the compound of formula (I) is a pharmaceutically acceptable salt and may include an acid addition salt or a salt with a base depending on the type of substituent. Examples, include but are not limited to, acid addition salts with inorganic acids, such as hydrochloric acid, hydrobromic acid, hydroiodic acid, sulfuric acid, nitric acid and phosphoric acid; acid addition salts with organic acids, such as formic acid, acetic acid, propionic acid, oxalic acid, malonic acid, succinic acid, fumaric acid, maleic acid, lactic acid, malic acid, mandelic acid, tartaric acid, dibenzoyl tartaric acid, ditoluoyl tartaric acid, citric acid, methanesulfonic acid, ethanesulfonic acid, benzenesulfonic acid, p-toluenesulfonic acid, aspartic acid, and glutaminic acid; salts with inorganic bases, such as sodium, potassium, magnesium, calcium and aluminum; and salts with organic bases, such as methylamine, ethylamine, ethanolamine, lysine and ornithine, salts with various amino acids, such as acetylleucine and amino acid derivatives, and ammonium salts.

Furthermore, the present invention also includes various hydrates, solvates, and polymorph of the compound of formula (I) and salt thereof.

The present invention also includes all pharmaceutically acceptable compounds of formula (I), or salts thereof labeled with one or more radioactive or non-radioactive isotopes. Examples of suitable isotopes used in the isotopic labeling of the compounds of the present invention include, but are not limited to, hydrogen (²H and ³H, etc.), carbon (¹¹C, ¹³C, and ¹⁴C, etc.), nitrogen (¹³N and ¹⁵N, etc.), oxygen (¹⁵O, ¹⁷O, and ¹⁸O, etc.), fluorine (¹⁸F, etc.), chlorine (³⁶Cl, etc.), iodine (¹²³I and ¹²⁵I, etc.), phosphorus (³²P, etc.), and sulfur (³⁵S, etc.). Isotope-labeled compounds of the present invention can be used for studies, such as drug and/or substrate tissue distribution studies. For example, radioactive isotopes, such as tritium (³H) and carbon-14 (¹⁴C), can be used for this purpose because of their case of labeling and convenience of detection.

Substitution with heavier isotopes, for example, substitution of hydrogen with deuterium (²H), can be therapeutically advantageous due to improved metabolic stability (for example, increased half-life in vivo, reduced dosage, reduced drug interaction). Substitution with positron emitting isotopes (¹¹C, ¹⁸F, ¹⁵O, and ¹³N, etc.) can be used in positron emission tomography (PET) tests to determine substrate receptor occupancy. Isotope-labeled compounds of the present invention are generally prepared by conventional methods known to a person skilled in the art or by the same methods as described in the Examples using appropriate isotope-labeled reagents in place of unlabeled reagents.

### (Preparation Method)

The compound of formula (I) and a salt thereof can be produced by applying various known synthetic methods based on the characteristics of the basic structure or the type of substituent. In some cases, depending on the type of substituent or functional group, it is effective to replace the functional group with an appropriate protecting group (a group that can be easily converted to the functional group) at the starting material to the intermediate stages. Examples of such protecting groups include, but are not limited to, the protecting groups described in “Greene's Protective Groups in Organic Synthesis (4th Edition, 2006)” by P. G. M. Wuts and T. W. Greene, which may be appropriately selected and used according to the method described herein. In such a method, a desired compound can be obtained by introducing the protecting group, carrying out the reaction, and then removing the protecting group, if necessary.

Further, the prodrug of the compound of formula (I) can be prepared by introducing a specific group at the starting material to the intermediate stages as in the case of the above-mentioned protecting group, or by performing a chemical reaction using the compound of formula (I). This reaction can be performed using a method well-known to a person skilled in the art, such as conventional esterification, amidation, or dehydration.

Hereinafter, a typical method for producing the compound of formula (I) is described. Each Preparation Method can also be performed with reference to the documents cited in the present specification. The Preparation Method of the present invention is not limited to the examples shown below.

The following abbreviations may be used herein.
HATU: 1-[bis(dimethylamino)methylene]-1H-1,2,3-triazolo[4,5-b]pyridinium 3-oxide hexafluorophosphate, DMF: N,N-dimethylformamide, THF: tetrahydrofuran, WSC: 1-ethyl-3-(3-dimethylaminopropyl) carbodiimide, COMU: N-[({[(1Z))-1-cyano-2-ethoxy-2-oxoethylidene]amino}oxy)(morpholin-4-yl)methylene]-N-methylmethanaminium hexafluorophosphate, DCC: N,N′-dicyclohexylcarbodiimide, CDI: 1,1′-carbonyl diimidazole, Xantphos: 4,5-bis(diphenylphosphino)-9,9-dimethylxanthene, Ruphos: 2-dicyclohexylphosphino-2′,6′-diisopropoxidephenyl, Xphos: 2-dicyclohexylphosphino-2′,4′,6′-triisopropylbiphenyl, BINAP: 2,2′-bis(diphenylphosphino)-1,1′-binaphthyl.

### Preparation Method 1

This Preparation Method is a method for producing, among the compounds of formula (I), a compound of formula (Ib) in which Z is —COOH, or a
a compound of formula (Ic) which is a salt thereof.

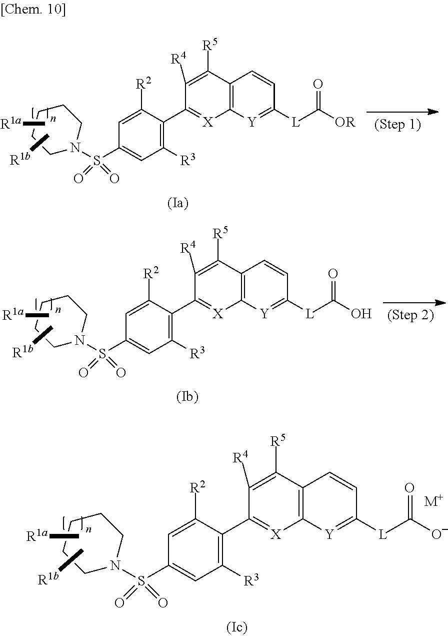

(wherein, R represents C₁₋₆ alkyl, and M⁺ represents a metal ion, particularly, but not limited to, Na⁺ or K⁺. Hereinafter, the same applies.)

### Step 1

This step is to obtain the compound of formula (Ib) by subjecting the compound of formula (Ia) to hydrolysis reaction conditions and neutralization.

This reaction is performed by using the compound of formula (Ia) and an excess amount of basic aqueous solution and stirring the mixture in a solvent inert to the reaction at room temperature to reflux condition for about 1 hour to about 1 day. After that, neutralization is performed with acid aqueous solution. The basic aqueous solution used here is not particularly limited, and examples include aqueous sodium hydroxide solution, aqueous potassium hydroxide solution, and aqueous lithium hydroxide solution. The solvent is not particularly limited, and examples include alcohols, such as methanol, ethanol and n-propanol, ether solvents, such as THF, diethyl ether, and 1,4-dioxane, and mixtures thereof. The acid aqueous solution used here is not particularly limited, and examples include an aqueous hydrochloric acid solution.

### Step 2

This step is to obtain the compound of formula (Ic) by subjecting the compound of formula (Ib) to salification reaction conditions.

This reaction is performed by using the compound of formula (Ib) and an excess amount of basic aqueous solution and stirring the mixture in a solvent inert to the reaction at 0 degrees Celsius to room temperature for about 1 hour to about 1 day. The basic aqueous solution used here is not particularly limited, and examples include aqueous sodium hydroxide solution and aqueous potassium hydroxide solution. The solvent is not particularly limited and examples, include ether solvents, such as THF, diethyl ether and 1,4-dioxane, alcohols, such as methanol, ethanol, and n-propanol, and toluene, and mixtures thereof.

### Preparation Method 2

This Preparation Method is a method for producing, among the compounds of formula (I), a compound of formula (Id) in which Z is —CONR^{Z1}R^{Z2}.

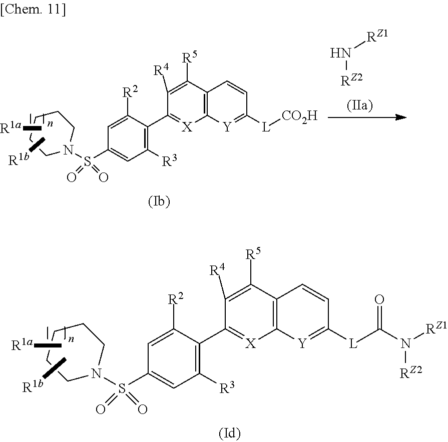

In this Preparation Method, the compound of formula (Id) is obtained by subjecting the compound of formula (Ib) and the compound of formula (IIa) to condensation reaction conditions.

This reaction is performed by using the compound of formula (Ib) obtained by Preparation Method 1 and the compound of formula (IIa) in equivalent or in excess amounts of either, adding a condensing agent and a base to the mixture, and stirring the mixture in a solvent inert to the reaction at room temperature for about 1 hour to about 1 day. The condensing agent used here is not particularly limited, and examples include HATU, WSC or its hydrochloride, DCC, CDI, COMU. The base is not particularly limited, and examples include organic bases, such as triethylamine, N,N-diisopropylethylamine and pyridine, and inorganic bases, such as potassium carbonate, sodium carbonate and cesium carbonate. The solvent is not particularly limited, and examples include halogenated hydrocarbons, such as dichloromethane, 1,2-dichloroethane, and chloroform, ether solvents, such as THF, diethyl ether, and 1,4-dioxane, alcohols, such as methanol, ethanol, and n-propanol, and DMF, and mixtures thereof. When the compound obtained by the condensation reaction has a protecting group, the compound of formula (Id) can be obtained by subjecting the compound following the condensation reaction to deprotection reaction conditions.

### Preparation Method 3

This Preparation Method is a method for producing, among the compounds of formula (I), a compound of formula (If) in which L and Z together form-O-(optionally substituted C₁₋₆ alkyl).

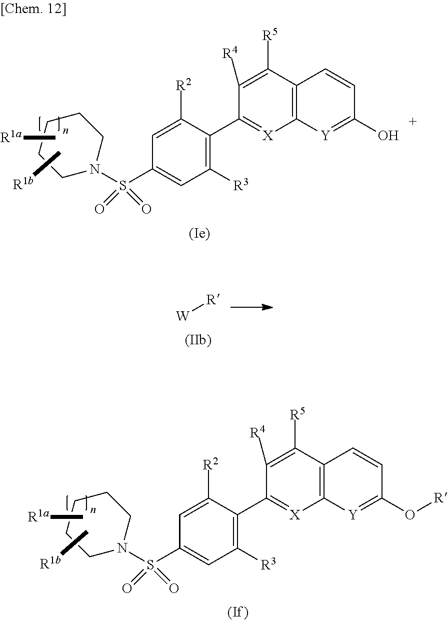

(wherein, R′ represents optionally substituted C₁₋₆ alkyl, and W represents halogen. Hereinafter, the same applies.)

In this Preparation Method, the compound of formula (If) is obtained by subjecting the compound of formula (Ie) to alkylation reaction conditions.

This reaction is performed by using the compound of formula (Ie) and the compound of formula (IIb) in equivalent or in excess amounts of either, adding a base to the mixture, and stirring the mixture in a solvent inert to the reaction at room temperature to about 100 degrees Celsius, preferably at about 60 degrees Celsius to about 100 degrees Celsius, under reflux for about 1 hour to about 1 day. The base used here is not particularly limited, and examples include inorganic bases, such as potassium carbonate, sodium carbonate, and cesium carbonate. The solvent is not particularly limited, and examples include ether solvents, such as THF and 1,4-dioxane, toluene, and DMF. When the compound obtained by the above-mentioned alkylation reaction has a protecting group, the compound of formula (If) can be obtained by subjecting the compound following the alkylation reaction to deprotection reaction conditions.

### Preparation Method 4

This Preparation Method is a method for producing, among compounds of formula (I), a compound of formula (Ih) in which L is a bond and Z is —NR^{Z1}R^{Z2}.

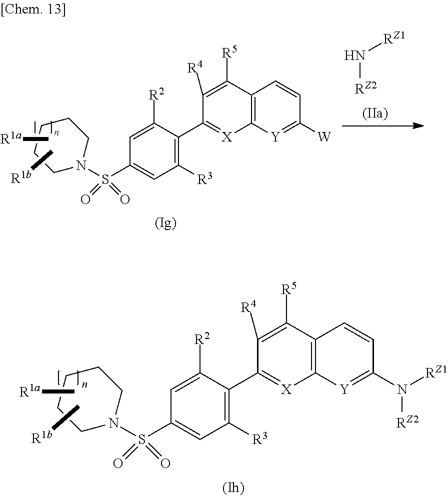

(wherein, W represents halogen. Hereinafter, the same applies.)

In this Preparation Method, the compound of formula (Ih) is obtained by subjecting the compound of formula (Ig) to carbon-nitrogen bond forming reaction conditions.

This reaction is performed by using the compound of formula (Ig) and the compound of formula (IIa) in equivalent or in excess amounts of either, adding a metal catalyst, a ligand, and a base to the mixture, and stirring the mixture in a solvent inert to the reaction at about 80 degrees Celsius to about 100 degrees Celsius under reflux condition for about 1 hour to about 1 day. The metal catalyst used here is not particularly limited, and examples include palladium acetate, tris(dibenzylideneacetone)dipalladium, [1,1′-bis(diphenylphosphino)ferrocene]palladium(II) dichloride, and [1,1′-bis(diphenylphosphino)ferrocene]palladium(II) dichloride dichloromethane adduct. The ligand is not particularly limited, and examples thereof include Xantphos, Ruphos, Xphos, BINAP. The base is not particularly limited, and examples include inorganic bases, such as potassium carbonate, sodium carbonate, cesium carbonate, and sodium tert-butoxide, and organic bases, such as triethylamine and N,N-diisopropylethylamine. The solvent is not particularly limited, and examples include 1,4-dioxane, toluene, DMF, and mixtures thereof. Further, this reaction may be performed under microwave irradiation. When the compound obtained by the carbon-nitrogen bond forming reaction has a protecting group, the compounds of formula (Ih) can be obtained by subjecting the compound following the carbon-nitrogen bond forming reaction to deprotection reaction conditions.

### Starting Material Synthesis 1

This Preparation Method is a method for producing a compound of formula (In) in which L is a bond, an exemplary compound of formula (Ia) used as a starting material in Preparation Method 1.

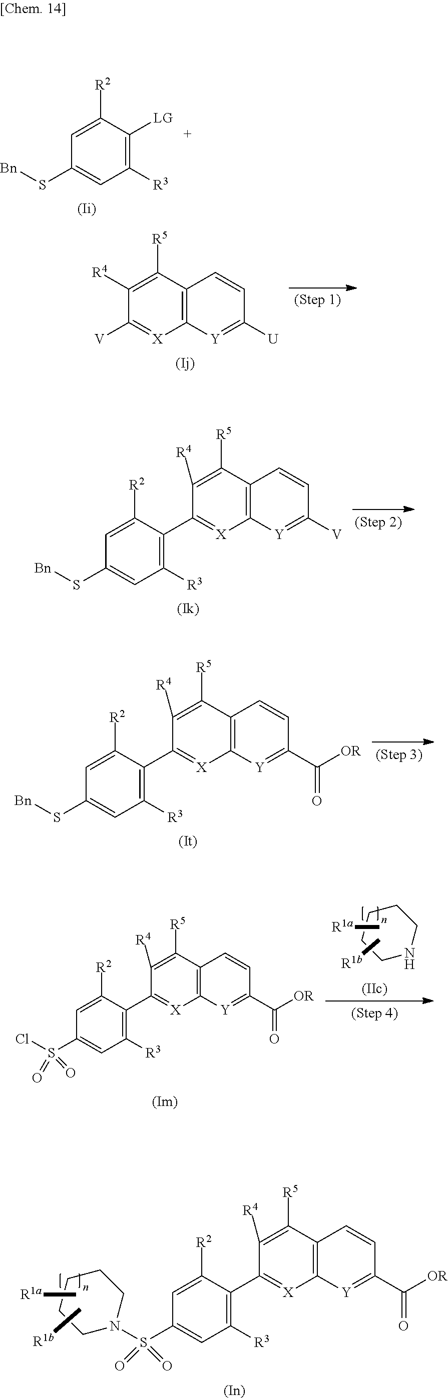

(wherein, LG represents boronic acid residue, boronate ester residue or potassium tri-fluoroborate residue. V represents halogen or trifluoromethanesulfonate residue. U represents halogen or hydroxy. Hereinafter, the same applies.)

### Step 1-1

This step is to obtain a compound of formula (Ik) by subjecting a compound of formula (Ii) and a compound of formula (Ij) to carbon-carbon bond forming reaction conditions.

This reaction is performed by using the compound of formula (Ii) and the compound of formula (Ij) in equivalent or in excess amounts of either, adding a metal catalyst and a base to the mixture, and stirring the mixture in a solvent inert to the reaction at room temperature to about 100 degrees Celsius, preferably at about 80 degrees Celsius to about 100 degrees Celsius, under reflux condition for about 1 hour to about 1 day. The metal catalyst used here is not particularly limited, and examples include tetrakis(triphenylphosphine)palladium (0), [1,1′-bis(diphenylphosphino)ferrocene]palladium(II) dichloride, and [1,1′-bis (diphenylphosphino)ferrocene]palladium(II) dichloride dichloromethane adduct. The base is not particularly limited, and examples include inorganic bases, such as potassium carbonate, cesium carbonate, and potassium phosphate, and organic bases, such as triethylamine and N,N′-diisopropylethylamine. The solvent is not particularly limited, and examples include ethers, such as 1,4-dioxane and THF, alcohols, such as ethanol and methanol, toluene, DMF, water and mixtures thereof. Further, this reaction may be performed under microwave irradiation.

### Step 1-2

When U is a hydroxy group, the compound of formula (Ik) is obtained by subjecting the compound after the above carbon-carbon bond forming reaction to triflation reaction conditions described below.

This reaction is performed by adding equivalent or excess amounts of triflating reagent and base to the compound obtained in the carbon-carbon bond forming reaction, and stirring the mixture in a solvent inert to the reaction at 0 degrees Celsius to room temperature for about 30 minutes to about 2 hours. The triflating reagent used here is not particularly limited, and examples include trifluoromethanesulfonic anhydride and N-phenylbis(trifluoromethanesulfonimide). The base is not particularly limited, and examples include organic bases, such as 2,6-lutidine, triethylamine, and N,N-diisopropylethylamine. The solvent is not particularly limited, and examples include halogenated hydrocarbons, such as dichloromethane, dichloroethane, and chloroform, ether solvents, such as THE, diethyl ether, and 1,4-dioxane, and toluene.

### Step 2

This step is to obtain a compound of formula (It) by subjecting a compound of formula (Ik) to carbon monoxide insertion reaction conditions.

This reaction is performed by using the compound of formula (Ik) and a metal catalyst, a ligand, a base, alcohol and carbon monoxide gas which is blown into the mixture, and stirring the mixture in a solvent inert to the reaction at about 90 degrees Celsius to reflux condition for about 2 hours to about 6 hours under carbon monoxide atmosphere. The metal catalyst used here is not particularly limited, and examples include palladium(II) acetate, tris(dibenzylideneacetone)dipalladium, [1,1′-bis(diphenyl phosphino)ferrocene]palladium(II) dichloride, and [1,1′-bis(diphenylphosphino)ferrocene]palladium(II) dichloride dichloromethane adduct. The ligand is not particularly limited, and examples thereof include 1,3-bis(dicyclohexyl phosphino) propane bis(tetrafluoroborate), and 1,1′-bis(diphenylphosphino)ferrocene. The base is not particularly limited, and examples include inorganic bases, such as potassium carbonate, sodium carbonate, and cesium carbonate, and organic bases, such as triethylamine,
N,N-diisopropylethylamine, and pyridine. The alcohol is not particularly limited, and examples include methanol and ethanol. The solvent is not particularly limited, and examples include DMF, dimethyl sulfoxide, or toluene.

### Step 3

This step is to obtain a compound of formula (Im) by subjecting a compound of formula (It) to chlorination reaction conditions.

This reaction is performed by using the compound of formula (It) and an excess amount of chlorinating reagent, and stirring the mixture in a solvent inert to the reaction at about 0 degrees Celsius to room temperature, preferably at room temperature, for about 1 hour to about 3 hours. The chlorinating reagent used here is not particularly limited, and examples include N-chlorosuccinimide, 1,3-dichloro-5,5-dimethylhydantoin, and sulfuryl chloride. The solvent is not particularly limited, and examples include acetic acid, water, acetone, and acetonitrile, and mixtures thereof.

### Step 4

This step is to obtain a compound of formula (In) by subjecting a compound of formula (Im) and a compound of formula (IIc) to sulfonamidation reaction conditions.

This reaction is performed by using the compound of formula (Im) and the compound of formula (IIc) in equivalent or in excess amounts of either, and stirring the mixture in the presence of a base in a solvent inert to the reaction at about 0 degrees Celsius to room temperature, preferably at room temperature, for about 1 hour to about 3 hours. The base used here is not particularly limited, and examples include organic bases, such as triethylamine and N,N-diisopropylethylamine, and inorganic bases, such as potassium carbonate, sodium carbonate, cesium carbonate, and sodium hydroxide. The solvent is not particularly limited, and examples include halogenated hydrocarbons, such as dichloromethane, dichloroethane, and chloroform, ether solvents, such as THF, diethyl ether, and 1,4-dioxane, and alcohol solvents, such as methanol, ethanol, and isopropanol, water, acetonitrile, toluene, and mixtures thereof.

### Starting Material Synthesis 2

This Preparation Method is a method for producing a compound of formula (Is) in which R⁴ is fluoro, V is trifluoromethanesulfonate, and X is N, an exemplary compound of formula (Ij) used in Starting material synthesis 1.

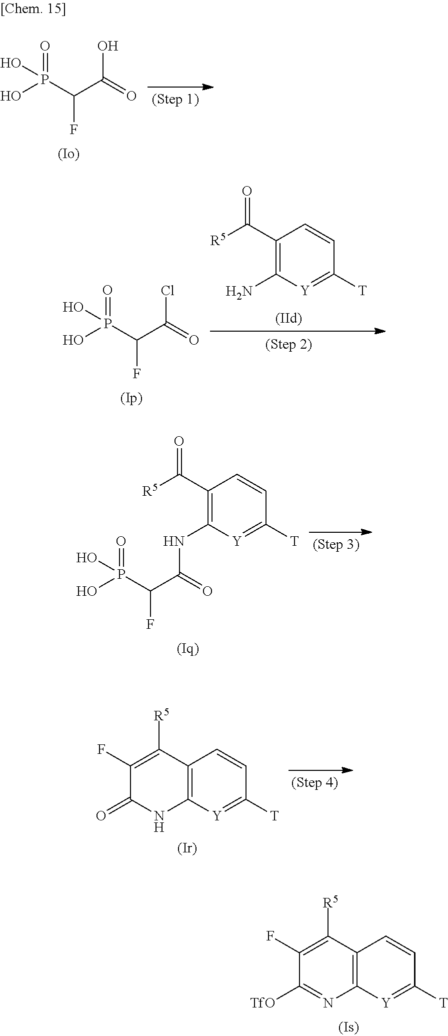

(wherein, T represents halogen. TfO represents trifluoromethanesulfonate. Hereinafter, the same applies.)

### Step 1

This step is to obtain a compound of formula (Ip) by subjecting a compound of formula (Io) to chlorination reaction conditions.

This reaction is performed by using the compound of formula (Io) and equivalent or excess amounts of a chlorinating reagent, and stirring the mixture in a solvent inert to the reaction at about 0 degrees Celsius to room temperature, preferably at room temperature, for about 12 hours to about 1 day. The chlorinating reagent used here is not particularly limited, and examples include oxalyl chloride and thionyl chloride. The solvent is not particularly limited, and examples include halogenated hydrocarbons, such as dichloromethane, dichloroethane, and chloroform, ether solvents, such as THF, diethyl ether, and 1,4-dioxane, toluene, and DMF.

### Step 2

This step is to obtain a compound of formula (Iq) by subjecting a compound of formula (Ip) and a compound of formula (IId) to acylation reaction conditions.

This reaction is performed by using the compound of formula (Ip) and the compound of formula (IId) in equivalent or in excess amounts of either, adding a base to the mixture, and stirring the mixture in a solvent inert to the reaction at about 0 degrees Celsius to room temperature, preferably at room temperature, for about 30 minutes to about 1 hour. The base used here is not particularly limited, and examples include organic bases, such as pyridine, triethylamine, and N,N-diisopropylethylamine, and inorganic bases, such as potassium carbonate, sodium carbonate, and cesium carbonate. The solvent is not particularly limited, and examples include halogenated hydrocarbons, such as dichloromethane, dichloroethane, and chloroform, ether solvents, such as THF, diethyl ether, and 1,4-dioxane, toluene, and DMF.

### Step 3

This step is to obtain a compound of formula (Ir) by subjecting a compound of formula (Iq) to cyclization reaction conditions.

This reaction is performed by stirring the compound of formula (Iq) with a base in a solvent inert to the reaction at about 0 degrees Celsius to room temperature condition, preferably at room temperature, for about 1 hour to about 3 hours. The base used here is not particularly limited, and examples include, inorganic bases, such as sodium hydride and potassium t-butoxide. The solvent is not particularly limited, and examples include ether solvents, such as THF, diethyl ether, and 1,4-dioxane, and toluene.

### Step 4

This step is to obtain a compound of formula (Is) by subjecting a compound of formula (Ir) to triflation reaction conditions.

This reaction is performed by stirring the compound of formula (Ir) with a triflating reagent and a base in a solvent inert to the reaction at about 0 degrees Celsius to room temperature, preferably at room temperature, for about 30 minutes to about 2 hours. The triflating reagent used here is not particularly limited, and examples include trifluoromethanesulfonic anhydride and N-phenylbis(trifluoromethanesulfonimide). The base is not particularly limited, and examples include organic bases, such as 2,6-lutidine, triethylamine, and N, N-diisopropylethylamine. The solvent is not particularly limited, and examples include halogenated hydrocarbons, such as dichloromethane, dichloroethane, and chloroform, ether solvents, such as THF, diethyl ether, and 1,4-dioxane, and toluene.

### Starting Material Synthesis 3

This Preparation Method is another method for producing a compound of formula (In) in which L is a bond, an exemplary compound of formula (Ia) used as a starting material in Preparation Method 1.

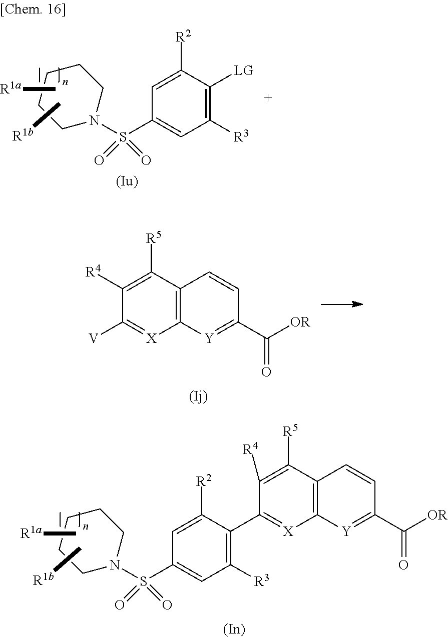

(wherein, R represents C₁₋₆ alkyl. Hereinafter, the same applies.)

### Step 1

The compound of the formula (In) is obtained from the compound formula (Iu) and (Ij) on the same reaction conditions as step 1-1 of Starting material synthesis 1.

### Starting Material Synthesis 4

This Preparation Method is a method for producing a compound of formula (Ix) in which LG is boronate ester, an exemplary compound of formula (Iu) used as a starting material in Starting material synthesis 3.

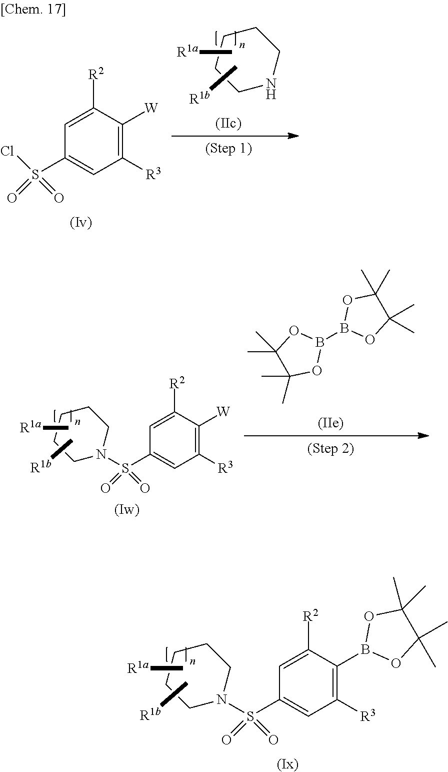

### Step 1

This step is to obtain a compound of formula (Iw) by subjecting a compound of formula (Iv) and corresponding amine (IIc) to sulfonamidation reaction condition. The compound of the formula (Iw) is obtained from the compound formula (Iv) on the same reaction conditions as step 4 of Starting material synthesis 1.

### Step 2

This step is to obtain a compound of formula (Ix) by subjecting a compound of formula (Iw) and a compound of formula (IIe) to Carbon-Boron bond forming reaction conditions.

This reaction is performed by using the compound of formula (Iw) and a compound of formula (IIe) in equivalent or in excess amounts of either, adding a metal catalyst and a base to the mixture, and stirring the mixture in a solvent inert to the reaction at about 60 degrees Celsius to about 100 degrees Celsius, preferably at about 80 degrees Celsius to about 100 degrees Celsius, under reflux condition for about 1 hour to about 1 day. The metal catalyst used here is not particularly limited, and examples include [1,1′-bis(diphenylphosphino)ferrocene]palladium(II) dichloride, and [1,1′-bis (diphenylphosphino)ferrocene]palladium(II) dichloride dichloromethane adduct. The base is not particularly limited, and examples include inorganic bases, such as potassium acetate, potassium carbonate, cesium carbonate, and potassium phosphate. The solvent is not particularly limited, and examples include ethers, such as 1,4-dioxane and THF, water and mixtures thereof. Further, this reaction may be performed under microwave irradiation.

The compound of formula (I) is isolated and purified as a free compound, a salt thereof, a hydrate of free compound or salt thereof, a solvate of free compound or salt thereof, or a polymorph of free compound or salt thereof. The salt of the compound of formula (I) can also be produced by subjecting it to a conventional salification reaction. Isolation and purification are performed by applying conventional chemical operations, such as extraction, fractional crystallization, and various fractional chromatography. Various isomers can be produced by selection of an appropriate starting material compound or by separation using the difference in physicochemical properties between the isomers. For example, an optical isomer can be obtained by a general optical resolution method of a racemate (for example, fractional crystallization leading to a diastereomeric salt with an optically active base or acid, chromatography using a chiral column, etc.) or produced from a suitable optically active starting material compound.

The pharmacological activity of the compound of formula (I) can be confirmed by the following test or its modified test which is apparent to the person skilled in the art. The following abbreviations may be used in the following examples herein.

ATCC: United States Cultured Cell Lineage Preservation Agency (American Type Culture Collection), DAPI: 4′,6-diamidino-2-phenylindole, DMEM: Dulbecco's Modified Eagle Medium, PBS: Phosphate Buffer Solution, PFA: Paraformaldehyde, FBS: Fetal Bovine Serum

### Test Example 1: Evaluation of the Induction of Acetylcholine Receptor Clustering in Mouse Myoblasts C2Cl2 Cells

Induction of acetylcholine receptor clustering by the test compound was evaluated by using fluorescently-labeled Bungarotoxin to image and quantify the acetylcholine receptor cluster region induced by the differentiation of myoblasts into myotubes, and measure the change in area. The following test method was optimized with reference to various reports including FEBS let., 586, 3111-3116 (2012). When a concentration response curve is obtained by plotting the logarithmic concentration of the test compound on the horizontal axis and the ratio of the area of acetylcholine receptor cluster region on the vertical axis, the inducing action of the test compound can be determined.

### (Test Method)

A concentration response curve as an index indicating acetylcholine receptor clustering inducing action of the test compound in mouse C2Cl2 cells was prepared. C2Cl2 cells (ATCC Number: CRL-1772) were seeded at about 5000 cells/well on a 384-well plate (354667; Corning) coated with type I collagen and allowed to incubate at 37 degrees Celsius overnight in DMEM+20% FBS+1% penicillin/streptomycin medium. The following day, the medium was removed and replaced with a differentiation medium containing DMEM+2% FBS+1% penicillin/streptomycin, and allowed to incubate at 37 degrees Celsius for 4 days. The test compound was dissolved in DMSO and added to a differentiation medium containing 0.1 ng/ml Agrin (550-AG-100; R&D systems) such that the final concentration was 0.0015 to 30 PM (3-fold serial dilution) or 0.00017 to 3.3 μM (3-fold serial dilution). The final concentration of DMSO was 0.3%. After the 4 days of incubation, the differentiation medium was replaced with a differentiation medium containing the test compound and 0.1 ng/ml Agrin or a differentiation medium containing 0.1 ng/ml Agrin or 10 ng/ml Agrin alone, and allowed to incubate at 37 degrees Celsius overnight. The next day, 10 μl of DMEM containing Bungarotoxin-Alexa488 (B13422; Life technologies) was added to the differentiation medium containing test compound and Agrin and Agrin alone so that the final concentration of Bungarotoxin-Alexa488 was 1 μg/ml, and was allowed to incubate at 37 degrees Celsius for 1 hour to label the acetylcholine receptor. After removing all the medium, 4% PFA was added to each well and allowed to incubate at room temperature for 10 minutes, then washed with PBS, and allowed to incubate at room temperature for 30 minutes in PBS containing 5% goat serum and 0.3% Triton X-100. After removing the PBS solution, PBS-Tween20 (28352; Thermo fisher scientific) containing Anti-Myosin Heavy Chain eFluor (registered trademark) 660 (50-6503-82; eBioscience) and DAPI (340-07971; Dojindo) was added to each well and allowed to incubate at room temperature for 1 hour to label the myotubes and nuclei. After washing three times with PBS-Tween20, the fluorescence was imaged using IN Cell Analyzer 6000 (GE Healthcare). The control groups did not have any test compound and (1) Agrin at a final concentration of 0.1 ng/ml and (2) Agrin at a final concentration of 10 ng/ml. The response of control group (1) was regarded as 0% and the response of the control group (2) was regarded as 100%. For quantification, the signal of only the acetylcholine receptor present in the myotube was used to eliminate non-specific fluorescence.

### (Evaluation of Activity)

EC₅₀ and Max value were used as indicators of activity in order to quantitatively evaluate the inducing action of the test compound on acetylcholine receptor clustering. EC₅₀ value is a test compound concentration which indicates 50% in terms of the area of acetylcholine receptor cluster region compared to that of 10 ng/ml Agrin (regarded as 100%) in the concentration response curve. EC₅₀ was calculated by nonlinear regression analysis from the concentration response curve using the area of the cluster region of the test compound as an index. Max value is the maximum response value. The Max value was indicated by the maximum response of the test compound in percentage in terms of the area of acetylcholine receptor cluster region compared to that of 10 ng/ml Agrin (regarded as 100%).

The results (EC₅₀ and Max value) of the example compounds of the present invention are shown in Tables 1 and 2 below. In Tables 1 and 2, Ex refers to the Example number described later. EC₅₀ of Ex 12, 15, 16, 52, 53, 56, 57, 60, 61, 63, 64 was calculated with molecular weight as monohydrochloride. EC₅₀ of Ex. 10, 13, 17, 58, 59, 62 was calculated with molecular weight as dihydrochloride. EC₅₀ of other Ex number was calculated with molecular weight as free compound.

TABLE 1

| Ex | EC₅₀(μM) | Max(%) |
|---|---|---|
| 1 | 0.064 | 91.9 |
| 3 | 0.002 | 91.8 |
| 4 | 0.013 | 81.0 |
| 5 | 0.095 | 86.8 |
| 6 | 1.819 | 76.3 |
| 7 | 1.851 | 78.4 |
| 8 | 0.045 | 88.2 |
| 9 | 34.9% at 1.1 μM | 34.9 |
| 10 | 0.016 | 96.7 |
| 11 | 0.303 | 80.2 |
| 12 | 0.268 | 69.7 |
| 13 | 0.003 | 117.1 |
| 14 | 0.021 | 83.5 |
| 15 | 0.002 | 105.2 |
| 35 | 0.020 | 99.5 |
| 36 | 0.392 | 80.2 |
| 37 | 3.224 | 70.1 |
| 38 | 0.724 | 78.5 |
| 39 | 2.574 | 78.2 |
| 40 | 0.177 | 80.0 |
| 41 | 0.052 | 87.0 |
| 42 | 0.019 | 78.1 |
| 43 | 0.021 | 91.6 |
| 44 | 0.005 | 81.8 |
| 45 | 0.009 | 86.1 |
| 46 | 0.043 | 80.9 |
| 47 | 0.428 | 102.3 |
| 48 | 3.748 | 74.0 |

TABLE 2

| Ex | EC₅₀(μM) | Max(%) |
|---|---|---|
| 16 | 0.045 | 91.1 |
| 17 | 0.065 | 78.7 |
| 18 | 0.003 | 99.8 |
| 19 | 0.016 | 108.3 |
| 20 | 0.013 | 86.5 |
| 21 | 0.147 | 97.3 |
| 22 | 1.903 | 84.8 |
| 23 | 0.069 | 85.9 |
| 24 | 0.011 | 92.3 |
| 25 | 0.182 | 104.8 |
| 26 | 0.019 | 99.8 |
| 27 | 0.103 | 94.6 |
| 28 | 4.028 | 74.2 |
| 29 | 46.2% at 30 μM | 46.2 |
| 30 | 1.121 | 94.7 |
| 31 | 48.8% at 30 μM | 48.8 |
| 32 | 38.8% at 30 μM | 38.8 |
| 33 | 0.065 | 86.1 |
| 34 | 0.016 | 94.1 |
| 49 | 1.090 | 81.9 |
| 50 | 3.518 | 70.5 |
| 51 | 0.159 | 77.8 |
| 52 | 0.033 | 94.2 |
| 53 | 0.096 | 77.9 |
| 54 | 1.486 | 60.8 |
| 55 | 7.165 | 38.9 |
| 56 | 0.053 | 97.0 |
| 57 | 0.089 | 87.0 |
| 58 | 0.033 | 89.6 |
| 59 | 0.049 | 95.8 |
| 60 | 0.011 | 99.2 |
| 61 | 0.026 | 92.4 |
| 62 | 0.001 | 86.4 |
| 63 | 0.165 | 103.7 |
| 64 | 0.024 | 107.0 |
| 65 | 0.053 | 84.0 |
| 66 | 0.010 | 88.4 |
| 67 | 0.015 | 94.3 |

As shown in Tables 1 and 2, concentration response curve was obtained in some of the compounds of the present invention using the area of cluster region as an index. From this, it was clarified that the compound of formula (I) has an inducing action of acetylcholine receptor clustering.

### Test Example 2: Suppressing Action on Muscle Weakness in MuSK Type Myasthenia Gravis Animal Model

In a MuSK-type myasthenia gravis animal model, in which the production of autoantibodies against endogenous MuSK was induced by immunizing with human recombinant MuSK protein, decreased grip strength was evaluated as a pathological index. A suppressing action on decrease in grip strength by administration of the test compound indicates that the test compound has a therapeutic effect.

### (Experimental Device)

A grip strength measuring device (GPM-100B; Melquest) was used to measure the grip strength of the extremities.

### (Test Method)

Repeated daily oral administration of the test compound in the MuSK-type myasthenia gravis animal model was performed as follows.

Seven point five micrograms of human MuSK recombinant protein (9810-MK; R&D systems) prepared with Freund's Complete Adjuvant (263810; Becton, Dickinson and Company) was intradermally administered to the tail of 8-week-old female DBA/2 mice (Charles River Laboratories Japan). Two weeks later, 7.5 micrograms of human MuSK recombinant protein prepared with Freund's Incomplete Adjuvant (263910; Becton, Dickinson and Company) was intradermally administered to the tail to induce myasthenia gravis mice. A week later, antibody titers against human MuSK in serum and grip strengths by the grip strength test were measured, and the mice were divided into groups of 6 mice each based on the antibody titer value and subjected to administration of the test compound. The test compound was orally administered to the mice in the treatment group (3, 10 and 30 mg/kg, suspended in 0.5% methylcellulose twice daily), and vehicle (0.5% methylcellulose) was used for the mice in the control group in place of the test compound. As a normal group, mice of the same age, same sex, and same strain were used that were not immunized with human recombinant MuSK protein. A grip strength test was performed after the administration in the morning of the 5th day.

### (Data Analysis)

The significance test between the normal group and the control group was performed by the Student-t test (*p<0.05). In addition, Dunnett's multiple comparison test was used between the control group and the test compound group (#p<0.05). A p-value less than 5% was regarded as significant in all the tests.

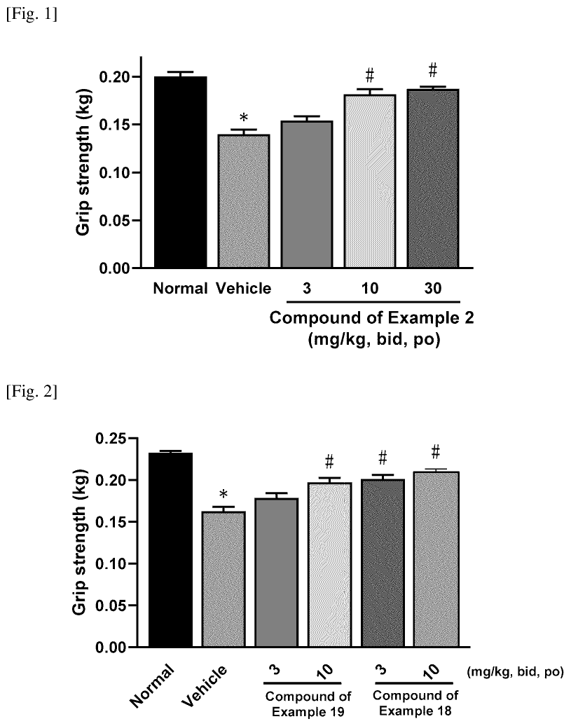

As shown in FIG. 1, the compound of Example 2 was found to have a suppressing action on grip strength decrease at 10 mg/kg and 30 mg/kg.

### Test Example 3: Suppressing Action on Muscle Weakness in MuSK Type Myasthenia Gravis Animal Model

In a MuSK-type myasthenia gravis animal model, in which the production of autoantibodies against endogenous MuSK was induced by immunizing with human recombinant MuSK protein, decreased grip strength was evaluated as a pathological index. A suppressing action on decrease in grip strength by administration of the test compound indicates that the test compound has a therapeutic effect.

### (Experimental Device)

A grip strength measuring device (GPM-100B; Melquest) was used to measure the grip strength of the extremities.

### (Test Method)

Repeated daily oral administration of the test compound in the MuSK-type myasthenia gravis animal model was performed as follows.

Seven point five micrograms of human MuSK recombinant protein (9810-MK; R&D systems) prepared with Freund's Complete Adjuvant (263810; Becton, Dickinson and Company) was intradermally administered to the tail of 8-week-old female DBA/2 mice (Charles River Laboratories Japan). Two weeks later, 7.5 micrograms of human MuSK recombinant protein prepared with Freund's Incomplete Adjuvant (263910; Becton, Dickinson and Company) was intradermally administered to the tail to induce myasthenia gravis mice. A week later, antibody titers against human MuSK in serum and grip strengths by the grip strength test were measured, and the mice were divided into groups of 6 mice each and subjected to administration of the test compound. The test compound was orally administered to the mice in the treatment group (3 and 10 mg/kg, suspended in 0.5% methylcellulose twice daily), and vehicle (0.5% methylcellulose) was used for the mice in the control group in place of the test compound. As a normal group, mice of the same age, same sex, and same strain were used that were not immunized with human recombinant MuSK protein. A grip strength test was performed after the administration in the morning of the 4th day.

### (Data Analysis)

The significance test between the normal group and the control group was performed by the Student-t test (*p<0.05). In addition, Dunnett's multiple comparison test was used between the control group and the test compound group (#p<0.05). A p-value less than 5% was regarded as significant in all the tests.

As shown in FIG. 2, the compound of Example 18 was found to have a suppressing action on grip strength decrease at 3 mg/kg and 10 mg/kg, the compound of Example 19 was found to have a suppressing action on grip strength decrease at 10 mg/kg.

Based in the foregoing, the compound of formula (I) or a salt thereof can be expected to be used for preventing and/or treating neuromuscular diseases.

A pharmaceutical composition comprising one or more of the compounds of formula (I) or a salt thereof as an active ingredient can be prepared by well-known methods using excipients commonly used in the art, that is, pharmaceutical excipients, pharmaceutical carriers, and the like.

Administration may be in any form, including oral administration by tablets, pills, capsules, granules, powders, liquids, or the like, or parenteral administration using injections, such as intra-articular, intravenous, and intramuscular, suppositories, eye drops, eye ointments, transdermal solutions, ointments, transdermal patches, transmucosal solutions, transmucosal patches, inhalants, or the like.

Solid compositions for oral administration, particularly, tablets, powders, granules, and the like are used. In such solid compositions, one or more active ingredients are mixed with at least one inert excipient. The composition may contain an inert additive, such as a lubricant, a disintegrant, a stabilizer, a solubilizing agent, in accordance with conventional methods. Tablets, powders, granules, or pills may optionally be coated, as necessary, with a film of wax, sugar coating, or gastric or enteric material.

Liquid compositions for oral administration contain pharmaceutically acceptable emulsions, solutions, suspensions, syrups, elixirs, or the like, and commonly used inert diluents such as purified water or ethanol. The liquid composition may contain auxiliary agents such as solubilizers, wetting agents, suspending agents, sweetening agents, flavoring agents, fragrance agents, and preservatives in addition to inert diluents.

Injections for parenteral administration contain sterile aqueous or non-aqueous solutions, suspensions, or emulsions. Examples of an aqueous solvent include, but are not limited to, distilled water for injection or physiological saline. Non-aqueous solvents include, but are not limited to, alcohols such as ethanol. Such compositions may further include tonicity agents, preservatives, wetting agents, emulsifiers, dispersants, stabilizers, or solubilizers. These are sterilized, for example, by filtration through a bacterial retention filter, formulation of fungicides, or irradiation. In addition, a sterile solid composition that is dissolved or suspended in sterile water or a sterile injectable solvent before use is also contemplated herein.

Examples of external preparations include ointments, plasters, creams, jellies, poultices, sprays, lotions, eye drops, and eye ointments, and commonly used ointment bases, lotion bases, aqueous or non-aqueous liquids, suspensions, and emulsions.

A transmucosal agent, such as an inhalant or a nasal agent, is used in the form of solid, liquid, or semi-solid, and can be produced in accordance with well-known methods. For example, known excipients, and further pH adjusters, preservatives, surfactants, lubricants, stabilizers, thickeners, and the like may be added thereto as appropriate.

In the case of oral administration, the normal daily dose is approximately 0.001 to 100 mg/kg body weight, preferably about 0.1 to about 30 mg/kg body weight, more preferably about 0.1 to about 10 mg/kg body weight, and administered once to 4 times per day in divided doses. When administered intravenously, the appropriate daily dose is approximately 0.0001 to 10 mg/kg body weight, and administered once to multiple times per day in divided doses.

The pharmaceutical composition of the present invention contains one or more compound of formula (I) or salt thereof as active ingredients at 0.01 to 100 wt %, and in some embodiments 0.01 to 50 wt %, which may vary depending on the route of administration, dosage form, administration site, type of excipient, or additive.

The compound of formula (I) can be used in combination with different agents for treating and/or preventing the disease for which the compound of formula (I) is effective. The combination may be administered simultaneously or separately, for example, consecutively or at desired time intervals. The simultaneously administered formulation may be a compounding agent or separately formulated.

### EXAMPLES

Hereinafter, a method for producing the compound of formula (I) will be described in more detail based on the Examples. The present invention is not limited to the compounds described in the following examples. Further, the Preparation Methods of the starting materials are shown as Preparation Examples. Further, the method for producing the compound of formula (I) is not limited to the Preparation Method of the specific examples shown below, and the compound of formula (I) can also be produced based on the combination of these Preparation Methods and/or other conventional methods known to a person skilled in the art.

In addition, the following abbreviations may be used in the Examples, Preparation Examples and Tables below.

PEx: Preparation Example number, Ex: Example number, PSyn: Preparation Example number produced by the same method, Syn: Example number produced by the same method, Str: Chemical structural formula, DAT: Physicochemical data, ESI+: m/z value in mass analysis (ionization method ESI, unless otherwise indicated [M+H]+), APCI/ESI+: APCI/ESI-MS (atmospheric pressure chemical ionization method APCI, APCI/ESI means simultaneous measurements of APCI and ESI. Unless otherwise indicated [M+H]+), API-ES+: API-ES MS (Atmospheric pressure ionization-electrospray method, unless otherwise indicated [M+H]+), J: Coupling constant, s: singlet, d: doublet, dd: double doublet, t: triplet, br: broad (example: brs), m: multiplet, rac: racemic mixture.

For convenience, the concentration mol/L is represented as M. For example, 1 M aqueous sodium hydroxide solution means 1 mol/L aqueous sodium hydroxide solution.

### Preparation Example 1

2-Bromo-1,3-difluoro-5-iodobenzene (15 g) was dissolved in 1,4-dioxane (150 mL), then phenylmethanethiol (5.7 mL), tris(dibenzylideneacetone)dipalladium (0) (2.2 g), xantphos (2.7 g), and N, N-diisopropylethylamine (16 mL) were added to the mixture and stirred at 90 degrees Celsius overnight under an argon atmosphere. After the mixture was allowed to cool to room temperature, hydrogen chloride (4 M in ethyl acetate, 24 mL) was added under ice-cooling, and the mixture was stirred at room temperature for 1 hour. The mixture was filtered through Celite and the filtrate was concentrated under reduced pressure to obtain the residue. Chloroform and basic silica gel were added to the obtained residue, and the mixture was stirred at room temperature for 1 hour. The mixture was filtered through Celite and washed with chloroform. The filtrate was concentrated under reduced pressure, and the obtained residue was purified by silica gel column chromatography (hexane/ethyl acetate) to obtain 5-(benzylsulfanyl)-2-bromo-1,3-difluorobenzene (13 g) as an oil.

### Preparation Example 2

To a mixture of methyl 3-fluoro-4-methyl-2-[(trifluoromethanesulfonyl)oxy]quinoline-7-carboxylate (410 mg), [4-(benzylsulfanyl)-2,6-difluorophenyl]boronic acid (380 mg), triethylamine (0.47 mL), and 1,4-dioxane (9 mL) was added tetrakis(triphenylphosphine)palladium (0) (130 mg), and the mixture was stirred at 100 degrees Celsius for 24 hours under an argon atmosphere. After the mixture was allowed to cool to room temperature, the mixture was concentrated under reduced pressure, and the resulting residue was purified by silica gel column chromatography (hexane/ethyl acetate) to obtain methyl 2-[4-(benzylsulfanyl)-2,6-difluorophenyl]-3-fluoro-4-methylquinoline-7-carboxylate (260 mg) as a solid.

### Preparation Example 3

To a mixture of 7-bromo-2-{2,6-difluoro-4-[(3S)-3-fluoropyrrolidine-1-sulfonyl]phenyl}-3-fluoro-4-methylquinoline (350 mg), tert-butyl 4-(4,4,5,5-tetramethyl-1,3,2-dioxaborolan-2-yl)-3,6-dihydropyridine-1 (2H)-carboxylate (330 mg), sodium carbonate (110 mg), 1,4-dioxane (8 mL), and water (2 mL) was added tetrakis(triphenylphosphine)palladium (0) (51 mg), and the mixture was stirred at 100 degrees Celsius for 24 hours under an argon atmosphere. After the mixture was allowed to cool to room temperature, water was added to the mixture, then the mixture was extracted with ethyl acetate. The organic layer was washed with brine and then dried over anhydrous magnesium sulfate, concentrated under reduced pressure, and the resulting residue was purified by silica gel column chromatography (hexane/ethyl acetate) to obtain the crude product. To the crude tert-butyl 4-(2-{2,6-difluoro-4-[(3S)-3-fluoropyrrolidine-1-sulfonyl]phenyl}-3-fluoro-4-methylquinolin-7-yl)-3,6-dihydropyridine-1 (2H)-carboxylate (440 mg) was added diisopropyl ether (9 mL) and the mixture was stirred at room temperature for 0.5 hours, then the solid was collected by filtration and dried under reduced pressure to obtain tert-butyl 4-(2-{2,6-difluoro-4-[(3S)-3-fluoropyrrolidine-1-sulfonyl]phenyl}-3-fluoro-4-methylquinolin-7-yl)-3,6-dihydropyridine-1 (2H)-carboxylate (380 mg) as a solid.

### Preparation Example 4

To a mixture of (3S)-1-(4-bromo-3,5-difluorobenzene-1-sulfonyl)-3-fluoropyrrolidine (50 mg), 1,4-dioxane (6 mL), and water (0.8 mL) was added [1,1′-bis (diphenylphosphino)ferrocene]dichloropalladium(II) dichloromethane adduct (12 mg), tert-butyl {2-[8-fluoro-5-methyl-7-(4,4,5,5-tetramethyl-1,3,2-dioxaborolan-2-yl) quinoline-2-carb oxamido]ethyl}(methyl)carbamate (60 mg), and potassium carbonate (50 mg), and the mixture was stirred at 100 degrees Celsius for 12 hours under an argon atmosphere. After the mixture was allowed to cool to room temperature, water and chloroform were added thereto to perform extraction, then the organic layer was concentrated. The obtained residue was purified by silica gel column chromatography (hexane/ethyl acetate) to obtain tert-butyl [2-(7-{2,6-difluoro-4-[(3S)-3-fluoropyrrolidine-1-sulfonyl]phenyl}-8-fluoro-5-methylquinoline-2-carboxamido)ethyl](methyl)carbamate (11 mg) as an oil.

### Preparation Example 5

A mixture of 1-(4-bromo-3-fluorobenzene-1-sulfonyl)-4-fluoropiperidine (800 mg), 4,4,4′,4′,5,5,5′,5′-octamethyl-2,2′-bi-1,3,2-dioxaborolane (720 mg), [1,1′-bis(diphenylphosphino)ferrocene]dichloropalladium(II) (86 mg), potassium acetate (280 mg), and 1,4-dioxane (8 mL) was stirred at 100 degrees Celsius for 1 hour under an argon atmosphere. The mixture was allowed to cool to room temperature, filtered through Celite, and the filtrate was concentrated under reduced pressure. The obtained residue was purified by silica gel column chromatography (hexane/ethyl acetate) to obtain 4-fluoro-1-[3-fluoro-4-(4,4,5,5-tetramethyl-1,3,2-dioxaborolan-2-yl)benzene-1-sulfonyl]piperidine (810 mg) as a solid.

### Preparation Example 6

To a mixture of tert-butyl 4-(7-bromo-5-methylquinolin-2-yl) piperazine-1-carboxylate (30 mg) and 1,4-dioxane (2 mL) was added 4,4,4′,4′,5,5,5′,5′-octamethyl-2,2′-bi-1,3,2-dioxaborolane (20 mg), [1,1′-bis(diphenylphosphino)ferrocene]dichloropalladium(II) dichloromethane adduct (8.0 mg) and potassium acetate (22 mg), and the mixture was stirred at 100 degrees Celsius overnight under an argon atmosphere. The mixture was allowed to cool to room temperature then water and ethyl acetate were added and the mixture was filtered through Celite. Water was added to the filtrate and the mixture was extracted with ethyl acetate. The organic layer was washed with brine, then dried over anhydrous sodium sulfate. Any insoluble material was filtered off and the filtrate was concentrated under reduced pressure. The obtained residue was purified by silica gel column chromatography (hexane/ethyl acetate) to obtain tert-butyl 4-[5-methyl-7-(4,4,5,5-tetramethyl-1,3,2-dioxaborolan-2-yl) quinolin-2-yl]piperazine-1-carboxylate (18 mg) as an oil.

### Preparation Example 7

5-(Benzylsulfanyl)-2-bromo-1,3-difluorobenzene (8.4 g) was dissolved in 1,4-dioxane (50 mL), 4,4,4′,4′,5,5,5′,5′-octamethyl-2,2′-bi-1,3,2-dioxaborolane (16 g), [1,1′-bis(diphenyl)phosphino)ferrocene]dichloropalladium(II) (3.9 g), and potassium acetate (6.3 g) were added, and the mixture was stirred at 100 degrees Celsius for 8 hours under an argon atmosphere. After the mixture was allowed to cool to room temperature, 4,4,4′,4′,5,5,5′,5′-octamethyl-2,2′-bi-1,3,2-dioxaborolane (4.1 g), [1,1′-bis(diphenylphosphino)ferrocene]dichloropalladium(II) (1.0 g) and potassium acetate (1.6 g) were added and stirred at 100 degrees Celsius for 16 hours under an argon atmosphere. After the mixture was allowed to cool to room temperature, 2-chloro-4-methylquinolin-7-ol (10 g), tetrakis(triphenylphosphine)palladium (0) (1.5 g), potassium carbonate (5.5 g), and water (9 mL) were added and stirred at 100 degrees Celsius for 24 hours under an argon atmosphere. After the mixture was allowed to cool to room temperature, chloroform and water were added thereto to perform extraction, and the organic layer was dried over anhydrous sodium sulfate. Any insoluble material was filtered off, the filtrate was concentrated, and the obtained residue was purified by silica gel column chromatography (chloroform/methanol, chloroform/ethyl acetate, hexane/ethyl acetate: using neutral silica gel at each purification) to obtain 2-[4-(benzylsulfanyl)-2,6-difluorophenyl]-4-methylquinolin-7-ol (2.4 g) as a solid, and 2-[4-(benzylsulfanyl)-2,6-difluorophenyl]-4-methylquinolin-7-ol (9.7 g) as an oil.

### Preparation Example 8

To a mixture of methyl 3-fluoro-4-methyl-2-oxo-1,2-dihydroquinoline-7-carboxylate (440 mg), 2,6-lutidine (0.44 mL), and dichloromethane (9 mL) was added trifluoromethanesulfonic anhydride (0.48 mL) under ice-cooling, and the mixture was stirred at the same temperature for 1.5 hours. A saturated aqueous sodium hydrogen carbonate solution was added to the mixture under ice-cooling. The mixture was extracted with chloroform, and the organic layer was dried over anhydrous sodium sulfate. Any insoluble material was filtered off, the filtrate was concentrated under reduced pressure, and the obtained residue was purified by silica gel column chromatography (hexane/ethyl acetate) to obtain methyl 3-fluoro-4-methyl-2-[(trifluoromethanesulfonyl)oxy]quinoline-7-carboxylate (410 mg) as a solid.

### Preparation Example 9

2-[4-(Benzylsulfanyl)-2,6-difluorophenyl]-4-methylquinolin-7-ol (2.4 g) was dissolved in dichloromethane (50 mL), then N, N-diisopropylethylamine (2.1 mL) and trifluoromethanesulfonic anhydride (1.5 mL) were added to the mixture under ice-cooling, and the mixture was stirred at room temperature for 16 hours. Chloroform and water were added to the mixture to perform extraction, and the organic layer was dried over anhydrous sodium sulfate. Any insoluble material was filtered off, the filtrate was concentrated, and the obtained residue was purified by silica gel column chromatography (hexane/ethyl acetate) to obtain 2-[4-(benzylsulfanyl)-2,6-difluorophenyl]-4-methylquinolin-7-yl trifluoromethanesulfonate (1.8 g) as a solid.

### Preparation Example 10

2-[4-(Benzylsulfanyl)-2,6-difluorophenyl]-4-methylquinolin-7-yl trifluoromethanesulfonate (1.8 g) was dissolved in DMF (18 mL) and methanol (9 mL), then palladium(II) acetate (77 mg), 1,1′-bis(diphenylphosphino)ferrocene (190 mg), and triethylamine (0.96 mL) were added to the mixture. Carbon monoxide was blown into the mixture for 5 minutes, then the mixture was stirred at 90 degrees Celsius for 3 hours under carbon monoxide atmosphere. After the mixture was allowed to cool to room temperature, water and ethyl acetate were added and the mixture was stirred for 10 minutes. Then, the insoluble material was filtered off through Celite and the filtrate was extracted with ethyl acetate. The organic layer was washed with brine and water (1:1) and dried over anhydrous sodium sulfate. Any insoluble material was filtered off, the filtrate was concentrated, and the obtained residue was purified by silica gel column chromatography (hexane/ethyl acetate, chloroform: using neutral silica gel at each purification) to obtain methyl 2-[4-(benzylsulfanyl)-2,6-difluorophenyl]-4-methylquinoline-7-carboxylate (440 mg) as an oil.

### Preparation Example 11

To a mixture of 3-bromo-2-fluoro-5-methylaniline (700 mg) and dichloromethane (10 mL) was added pyridine (0.54 mL) and (2E)-3-ethoxyprop-2-enoyl chloride (0.61 mL) under ice-cooling and the mixture was stirred at room temperature overnight under an argon atmosphere. To the mixture were added 1 M hydrochloric acid and water, the mixture was extracted with chloroform, and the organic layer was washed with brine then dried over anhydrous sodium sulfate. Any insoluble material was filtered off, and the filtrate was concentrated under reduced pressure. The obtained residue was purified by silica gel column chromatography (hexane/ethyl acetate) to obtain (2E)-N-(3-bromo-2-fluoro-5-methylphenyl)-3-ethoxyprop-2-enamide (700 mg) as a solid.

### Preparation Example 12

To a mixture of 4-bromo-3,5-difluorobenzene-1-sulfonyl chloride (11 g), N,N-diisopropylethylamine (15 mL), and dichloromethane (110 mL) was added 4-fluoropiperidine monohydrochloride (5.1 g) under ice-cooling and the mixture was stirred at room temperature for 1 hour. The mixture was concentrated then chloroform and water were added to the obtained residue to perform extraction, and the organic layer was dried over anhydrous magnesium sulfate, concentrated, and the obtained residue was dissolved in ethyl acetate and chloroform, silica gel was added to the solution. Any insoluble material was filtered off through Celite and the insoluble material was washed with ethyl acetate. The combined filtrate was concentrated, ethyl acetate (25 mL) was added to the obtained residue, and the mixture was stirred at room temperature for 0.5 hours. The obtained solid was collected by filtration and dried to obtain 1-(4-bromo-3,5-difluorobenzene-1-sulfonyl)-4-fluoropiperidine (10 g) as a solid.

### Preparation Example 13

Methyl 2-[4-(benzylsulfanyl)-2,6-difluorophenyl]-3-fluoro-4-methylquinoline-7-carboxylate (510 mg) was suspended in acetic acid (8 mL) and water (2 mL), then N-chlorosuccinimide (0.62 g) was added under ice-cooling, and the mixture was stirred at room temperature for 55 minutes. Ice was added to the mixture at room temperature and stirred until the ice was dissolved, then the solid was collected by filtration. The solid was dissolved in chloroform to perform extraction, and the organic layer was dried over anhydrous magnesium sulfate, concentrated under reduced pressure. Dichloromethane (7.5 mL) and triethylamine (0.47 mL) were added to the obtained residue, then (3S)-3-fluoropyrrolidine monohydrochloride (0.15 g) was added to the mixture, and the mixture was stirred at room temperature for 40 minutes. Water was added to the mixture and separated into two layers-aqueous and organic layers, and the organic layer was concentrated under reduced pressure. The obtained residue was purified by silica gel column chromatography (hexane/ethyl acetate, chloroform/methanol: using neutral silica gel for both) to obtain methyl 2-{2,6-difluoro-4-[(3S)-3-fluoropyrrolidine-1-sulfonyl]phenyl}-3-fluoro-4-methylquinoline-7-carboxylate (340 mg) as a solid.

### Preparation Example 14

1-(4-Bromo-3,5-difluorobenzene-1-sulfonyl)-4-fluoropiperidine (11 g) was dissolved in dehydrated THF (100 mL), then n-butyllithium (1.6 M in n-hexane, 20 mL) was added to the mixture under dry ice-acetone cooling under an argon atmosphere, and then the mixture was stirred at the same temperature for 15 minutes. Zinc chloride (4.2 g) was added to the mixture and stirred at the same temperature for 0.5 hours, then warmed to room temperature, and further stirred for 0.5 hours. Methyl 2-chloro-4-methylquinoline-7-carboxylate (6 g) and tetrakis(triphenylphosphine)palladium (0) (3 g) were added to the mixture and stirred at 60 degrees Celsius for 5 hours under an argon atmosphere. After the mixture was allowed to cool to room temperature, 1 M hydrochloric acid was added to the mixture, then water was added, and the mixture was extracted with ethyl acetate. The organic layer was washed with brine, then dried over anhydrous magnesium sulfate, concentrated under reduced pressure, and the obtained residue was purified by silica gel column chromatography (hexane/ethyl acetate) to obtain methyl 2-[2,6-difluoro-4-(4-fluoropiperidine-1-sulfonyl)phenyl]-4-methylquinoline-7-carboxy late (8.4 g) as a solid.

### Preparation Example 15

2-Chloro-4-methylquinoline-7-carboxylic acid (20 g) was dissolved in DMF (100 mL), then potassium carbonate (17 g) and iodomethane (9 mL) were added to the mixture under ice-cooling and stirred at room temperature for 0.5 hours. DMF (50 mL) was added to the mixture and stirred at room temperature for 5 hours. The mixture was ice-cooled, then ice water (200 mL) was added to the mixture, and the solid was collected by filtration, washed with water, and dried under reduced pressure. Acetonitrile (200 mL) was added to the obtained solid, and the mixture was stirred for 0.5 hours. Then, the solid was collected by filtration and dried under reduced pressure to obtain methyl 2-chloro-4-methylquinoline-7-carboxylate (15 g) as a solid.

### Preparation Example 16

5-(Benzylsulfanyl)-2-bromo-1,3-difluorobenzene (13 g) was dissolved in THF (130 mL), then n-butyl lithium (1.6 M in n-hexane, 31 mL) was added to the mixture under dry ice-methanol cooling under an argon atmosphere, and the mixture was stirred at the same temperature for 30 minutes. Trimethyl borate (6.8 mL) was added to the mixture at the same temperature, then the mixture was warmed to room temperature and stirred for 2 hours. To the mixture was added 1 M hydrochloric acid (180 mL) under ice-cooling, and the mixture was stirred at room temperature for 30 minutes. Water was added to the reaction mixture and the mixture was extracted with ethyl acetate. The organic layer was dried over anhydrous magnesium sulfate. Any insoluble material was filtered off, the filtrate was concentrated under reduced pressure. Acetonitrile was added to the obtained residue. The solid was collected by filtration, and dried under reduced pressure to obtain [4-(benzylsulfanyl)-2,6-difluorophenyl]boronic acid (6.5 g) as a solid.

### Preparation Example 17

To a mixture of (diethoxyphosphoryl) (fluoro) acetic acid (6.5 g), oxalyl chloride (10 mL), and dichloromethane (60 mL) was added DMF (0.04 mL) under ice-cooling and stirred at room temperature for 1.5 hours. The mixture was concentrated under reduced pressure, then toluene was added and the mixture was concentrated under reduced pressure again to obtain the residue. A mixture of the resulting residue and dichloromethane (30 mL) was added dropwise to a mixture of methyl 4-acetyl-3-aminobenzoate (4.1 g), pyridine (4 mL) and dichloromethane (30 mL) under ice-cooling. Afterwards, the mixture was warmed to room temperature and the mixture was stirred. After adding 1 M hydrochloric acid (50 mL) to the mixture under ice-cooling, the mixture was extracted with chloroform, and the organic layer was dried over anhydrous magnesium sulfate, concentrated under reduced pressure. Ethyl acetate (50 mL) was added to the obtained residue to triturate, and the mixture was stirred at room temperature for 1 hour. The solid was collected by filtration and dried under reduced pressure to obtain methyl 4-acetyl-3-[2-(diethoxyphosphoryl)-2-fluoroacetamido]benzoate (6.6 g) as a solid.

### Preparation Example 18

A mixture of methyl 4-acetyl-3-[2-(diethoxyphosphoryl)-2-fluoroacetamido]benzoate (1.0 g) and THF (20 mL) was added over 3 minutes to a mixture of sodium hydride (60% oily, 220 mg) and THF (10 mL) under water-cooling in an argon atmosphere then stirred at room temperature for 1.5 hours. A saturated aqueous ammonium chloride solution and water were added to the mixture under ice-cooling, and the mixture was concentrated under reduced pressure. After adding THF to the obtained residue, the mixture was stirred under ice-cooling for 45 minutes. The solid was collected by filtration and dried under reduced pressure to obtain methyl 3-fluoro-4-methyl-2-oxo-1,2-dihydroquinoline-7-carboxylate (440 mg) as a solid.

### Preparation Example 19

Methyl 2-{2,6-difluoro-4-[(3S)-3-fluoropyrrolidine-1-sulfonyl]phenyl}-3-fluoro-4-methylquinoline-7-carboxylate (380 mg) was suspended in THF (6 mL), and the mixture was ice-cooled under an argon atmosphere. Diisobutylaluminum hydride (1 M in toluene, 3 mL) was added to the mixture and stirred at room temperature for 1 hour. The reaction mixture was ice-cooled, then 1 M aqueous potassium sodium tartrate solution was added dropwise to the mixture, after ethyl acetate was added, and the mixture was stirred at room temperature for 30 minutes. Sodium potassium tartrate, water, and ethyl acetate were added to the mixture and stirred at room temperature for 1 hour. The mixture was extracted with ethyl acetate, and the organic layer was washed with brine, then dried over anhydrous magnesium sulfate, concentrated under reduced pressure, and the obtained residue was purified by silica gel column chromatography (hexane/ethyl acetate) to obtain (2-{2,6-difluoro-4-[(3S)-3-fluoropyrrolidine-1-sulfonyl]phenyl}-3-fluoro-4-methylquinolin-7-yl) methanol (350 mg) as a solid.

### Preparation Example 20

A mixture of 3-(2-{2,6-difluoro-4-[(3S)-3-fluoropyrrolidine-1-sulfonyl]phenyl}-3-fluoro-4-methylquinolin-7-yl) propanenitrile (110 mg) and dichloromethane (4 mL) was ice-cooled under an argon atmosphere, then diisobutylaluminum hydride (1 M in toluene, 350 microliters) was added and the mixture was stirred under ice-cooling for 40 minutes. Diisobutylaluminum hydride (1 M in toluene, 250 microliters) was added to the mixture under ice-cooling, and the mixture was stirred under ice-cooling for 30 minutes. To the mixture was added a 10% aqueous citric acid solution, water, and ethyl acetate under ice-cooling, and the mixture was stirred at room temperature overnight. The mixture was extracted with ethyl acetate, and the organic layer was washed with brine then dried over anhydrous magnesium sulfate, concentrated under reduced pressure, and the obtained residue was purified by silica gel column chromatography (hexane/ethyl acetate) to obtain 3-(2-{2,6-difluoro-4-[(3S)-3-fluoropyrrolidine-1-sulfonyl]phenyl}-3-fluoro-4-methylquinolin-7-yl) propanal (61 mg) as a solid.

### Preparation Example 21

(2-{2,6-Difluoro-4-[(3S)-3-fluoropyrrolidine-1-sulfonyl]phenyl}-3-fluoro-4-methylquinolin-7-yl) methanol (350 mg) was suspended in dichloromethane (7.5 mL), then 1,1,1-triacetoxy-1,1-dihydro-1,2-benziodoxol-3-(1H)-one (500 mg) was added to the mixture under ice-cooling and stirred under ice-cooling for 1 hour. A saturated aqueous sodium hydrogen carbonate solution was added to the mixture under ice-cooling, then the mixture was extracted with chloroform, and the organic layer was dried over anhydrous magnesium sulfate, concentrated under reduced pressure, and the obtained residue was purified by silica gel column chromatography (hexane/ethyl acetate) to obtain 2-{2,6-difluoro-4-[(3S)-3-fluoropyrrolidine-1-sulfonyl]phenyl}-3-fluoro-4-methylquinoline-7-carbaldehyde (330 mg) as a solid.

### Preparation Example 22

A mixture of sodium hydride (60% oily, 63 mg) and THF (3 mL) was ice-cooled under an argon atmosphere, then diethyl cyanomethylphosphonate (260 microliters) was added to the mixture, and the mixture was stirred at room temperature for 30 minutes. The mixture was ice-cooled and a mixture of 2-{2,6-difluoro-4-[(3S)-3-fluoropyrrolidine-1-sulfonyl]phenyl}-3-fluoro-4-methylquinoline-7-carbaldehyde (330 mg) and THF (9 mL) was added, and the mixture was stirred at room temperature for 30 minutes. Water was added to the mixture under ice-cooling, then the mixture was extracted with ethyl acetate and the organic layer was dried over anhydrous magnesium sulfate, concentrated under reduced pressure, and the obtained residue was purified by silica gel column chromatography (hexane/ethyl acetate) to obtain (2E)-3-(2-{2,6-difluoro-4-[(3S)-3-fluoropyrrolidine-1-sulfonyl]phenyl}-3-fluoro-4-methylquinolin-7-yl) prop-2-enenitrile (360 mg) as a solid.

### Preparation Example 23

(2E)-3-(2-{2,6-Difluoro-4-[(3S)-3-fluoropyrrolidine-1-sulfonyl]phenyl}-3-fluoro-4-methylquinolin-7-yl) prop-2-enenitrile (210 mg) was dissolved in THF (3 mL) and ethanol (3 mL), then 10% palladium carbon (50% water content, 49 mg) was added to the mixture under an argon atmosphere. After replacing argon with hydrogen, the mixture was stirred under normal pressure at room temperature for 3 hours. Celite was added to the mixture and any insoluble material was filtered off. The filtrate was concentrated under reduced pressure, and the obtained residue was purified by silica gel column chromatography (hexane/ethyl acetate) to obtain 3-(2-{2,6-difluoro-4-[(3S)-3-fluoropyrrolidine-1-sulfonyl]phenyl}-3-fluoro-4-methylquinolin-7-yl) propanenitrile (220 mg) as a solid.

### Preparation Example 24

The reaction was conducted in two batches. To a mixture of 7-bromo-8-fluoro-2-{2-fluoro-4-[(3S)-3-fluoropyrrolidine-1-sulfonyl]phenyl}-4-methylquinoline (150 mg) and tert-butyl 3-oxopiperazine-1-carboxylate (120 mg) in 1,4-dioxane (3 mL) were added cesium carbonate (200 mg), Xantphos (36 mg) and palladium acetate (10 mg), and it was bubbled with nitrogen for 2 minutes, then sealed in a tube. The mixture was heated in the microwave for 35 minutes at 140 degrees Celsius. The mixture was filtered, and the filtrate was concentrated under reduced pressure, and the obtained residue was purified by silica gel column chromatography (ethyl acetate/petroleum ether) to obtain the crude product. The crude product was further purified by preparative thin layer chromatography (petroleum ether/ethyl acetate) to obtain tert-butyl 4-(8-fluoro-2-{2-fluoro-4-[(3S)-3-fluoropyrrolidine-1-sulfonyl]phenyl}-4-methylquinolin-7-yl)-3-oxopiperazine-1-carboxylate (70 mg) as an oil.

### Preparation Example 25

To a mixture of 2-{2-fluoro-4-[(3S)-3-fluoropyrrolidine-1-sulfonyl]phenyl}-4-methylquinolin-7-ol (1.0 g) and DMF (10 mL) was added tert-butyl-N-(2-bromoethyl) carbamate (740 mg) and potassium carbonate (700 mg), and then stirred at 100 degrees Celsius for 3 hours. tert-Butyl-N-(2-bromoethyl) carbamate (170 mg) was added to the mixture and stirred at 100 degrees Celsius for 6 hours. After the mixture was allowed to cool to room temperature, ethyl acetate and water were added thereto to perform extraction, and the organic layer was dried over anhydrous magnesium sulfate. Any insoluble material was filtered off, the filtrate was concentrated under reduced pressure, and the obtained residue was purified by silica gel column chromatography (hexane/ethyl acetate, chloroform/methanol: using neutral silica gel for both) to obtain tert-butyl {2-[(2-{2-fluoro-4-[(3S)-3-fluoropyrrolidine-1-sulfonyl]phenyl}-4-methylquinolin-7-yl)oxy]ethyl}carbamate (930 mg) as a solid.

### Preparation Example 26

A mixture of {2-bromo-5-[(3S)-3-fluoropyrrolidine-1-sulfonyl]phenyl}methanol (0.21 g), 3,4-dihydro-2H-pyran (100 microliters), pyridinium p-toluenesulfonate (15 mg), and dichloromethane (3 mL) was stirred at room temperature for 3 hours. Water was added to the mixture, and the mixture was extracted with chloroform. The organic layer was concentrated under reduced pressure and the obtained residue was purified by silica gel column chromatography (hexane/ethyl acetate) to obtain (3S)-1-(4-bromo-3-{[(oxan-2-yl)oxy]methyl}benzene-1-sulfonyl)-3-fluoropyrrolidine (0.24 g) as a solid.

### Preparation Example 27

To a mixture of methyl 2-(4-[(3S)-3-fluoropyrrolidine-1-sulfonyl]-2-{[(oxan-2-yl)oxy]methyl}phenyl)-4-methylquinoline-7-carboxylate (180 mg), THF (2 mL), and methanol (2 mL) was added 1 M hydrochloric acid (1 mL), and the mixture was stirred overnight. A saturated aqueous sodium hydrogen carbonate solution and ethyl acetate were added to the mixture to perform extraction, and the organic layer was dried over anhydrous magnesium sulfate. Any insoluble material was filtered off, and the filtrate was concentrated under reduced pressure to obtain methyl 2-{4-[(3S)-3-fluoropyrrolidine-1-sulfonyl]-2-(hydroxymethyl)phenyl}-4-methylquinoline-7-carboxylate (150 mg) as a solid.

### Preparation Example 28

To a mixture of 2-bromo-5-[(3S)-3-fluoropyrrolidine-1-sulfonyl]benzoic acid (440 mg) and THF (10 mL) was added borane-THF complex (0.91 M in THF, 4 mL) under ice-cooling, then the mixture was warmed to room temperature and stirred overnight. Water and 1 M hydrochloric acid were added to the reaction mixture under ice-cooling and stirred, and then extracted with ethyl acetate. The organic layer was dried over anhydrous magnesium sulfate, and any insoluble material was filtered off and the filtrate was concentrated under reduced pressure. The obtained residue was purified by silica gel column chromatography (hexane/ethyl acetate) to obtain {2-bromo-5-[(3S)-3-fluoropyrrolidine-1-sulfonyl]phenyl}methanol (270 mg) as a solid.

### Preparation Example 29

A mixture of 7-bromo-2-{2,6-difluoro-4-[(3S)-3-fluoropyrrolidine-1-sulfonyl]phenyl}-4-methylquinoline (200 mg), 1-(tert-butyldimethylsilyloxy)-1-methoxyethene (0.18 mL), tris(dibenzylideneacetone)dipalladium (0) (38 mg), tri-tert-butylphosphine (0.02 mL), triethylamine (0.12 mL), and DMF (2 mL) was stirred at 120 degrees Celsius for 0.5 hours under microwave irradiation. After the mixture was allowed to cool to room temperature, water was added and the mixture was extracted with ethyl acetate. The organic layer was washed with 1 M hydrochloric acid and a saturated aqueous sodium hydrogen carbonate solution, and then dried over anhydrous magnesium sulfate. Any insoluble material was filtered off, the filtrate was concentrated under reduced pressure, and the obtained residue was purified by silica gel column chromatography (hexane/ethyl acetate: using basic silica gel) to obtain methyl (2-{2,6-difluoro-4-[(3S)-3-fluoropyrrolidine-1-sulfonyl]phenyl}-4-methylquinolin-7-yl) acetate (65 mg) as an oil.

### Preparation Example 30

7-[4-(Benzylsulfanyl)-2,6-difluorophenyl]-5-methyl-1,8-naphthyridine-2-carbonitrile (3.7 g) was suspended in methanol (30 mL), then sodium methoxide (5 M in methanol, 4.4 mL) was added and the mixture was stirred at room temperature for 5 hours. To the mixture was added 6 M hydrochloric acid (8.8 mL) under ice-cooling, and the mixture was stirred at room temperature for 0.5 hours. A saturated aqueous sodium hydrogen carbonate solution and ethyl acetate were added to the mixture under ice-cooling, the mixture was concentrated under reduced pressure. The obtained residue was extracted with ethyl acetate, and the organic layer was washed with brine, and then dried over anhydrous magnesium sulfate, concentrated under reduced pressure. The obtained residue was dissolved in ethyl acetate (10 mL). Diethyl ether (5 mL) was added thereto, and the mixture was stirred overnight at room temperature. The resulting solid was collected by filtration and dried under reduced pressure to obtain methyl 7-[4-(benzylsulfanyl)-2,6-difluorophenyl]-5-methyl-1,8-naphthyridine-2-carboxylate (3.6 g) as a solid.

### Preparation Example 31

To a mixture of 2-[4-(benzylsulfanyl)-2,6-difluorophenyl]-7-chloro-4-methyl-1,8-naphthyridine (5.9 g) and DMF (60 mL) was added zinc cyanide (3.5 g) and tetrakis(triphenylphosphine)palladium (0) (1.7 g), and the mixture was stirred at 120 degrees Celsius for 1.5 hours under an argon atmosphere. The mixture was allowed to cool to room temperature and concentrated under reduced pressure. Basic silica gel (120 mL) and chloroform (120 mL) was added to the mixture of the obtained residue, and the mixture was stirred at room temperature for 0.5 hours. Any insoluble material was filtered off, washed with chloroform, and the filtrate was concentrated under reduced pressure, and the obtained residue was purified by silica gel column chromatography (chloroform) to obtain the crude product. Ethyl acetate (35 mL) was added to the obtained crude 7-[4-(benzylsulfanyl)-2,6-difluorophenyl]-5-methyl-1,8-naphthyridine-2-carbonitrile (5.8 g) and the mixture was stirred for 4 hours at room temperature. The solid was collected by filtration and dried under reduced pressure to obtain 7-[4-(benzylsulfanyl)-2,6-difluorophenyl]-5-methyl-1,8-naphthyridine-2-carbonitrile (3.7 g) as a solid.

### Preparation Example 32

tert-Butyl 3-[6-chloro-2-(2,2-dimethylpropanamido)pyridin-3-yl]-3-hydroxybutanoate (27 g) was dissolved in THF (160 mL), then 3 M hydrochloric acid (160 mL) was added to the mixture and stirred at 90 degrees Celsius for 2 days. To the mixture was added 3 M hydrochloric acid (80 mL) and the mixture was stirred at 90 degrees Celsius for 2 hours. A saturated aqueous sodium hydrogen carbonate solution and 1 M aqueous sodium hydroxide solution were added to the mixture to perform neutralization, and then the mixture was stirred under ice-cooling for 0.5 hours. The solid was collected by filtration and dried under reduced pressure. The obtained solid was suspended in and washed with acetonitrile. The solid was collected by filtration and dried under reduced pressure to obtain 7-chloro-4-methyl-1,8-naphthyridin-2-ol (11 g) as a solid.

### Preparation Example 33

tert-Butyl acetate (24 mL) was added dropwise to a mixture of lithium hexamethyldisilazide (1.3 M in THF, 140 mL) and THF (150 mL) under dry ice-acetone cooling under an argon atmosphere and the mixture was stirred for 1 hour at the same temperature. A mixture of N-(3-acetyl-6-chloropyridin-2-yl)-2,2-dimethylpropanamide (21 g) and THF (50 mL) was added dropwise to the mixture, and the mixture was stirred at room temperature for 1 hour. Water was added to the mixture under ice-cooling, then the mixture was extracted with ethyl acetate, and the organic layer was dried over anhydrous magnesium sulfate. Any insoluble material was filtered off, the filtrate was concentrated under reduced pressure, and the obtained residue was purified by silica gel column chromatography (hexane/ethyl acetate) to obtain tert-butyl 3-[6-chloro-2-(2,2-dimethylpropanamido)pyridin-3-yl]-3-hydroxybutanoate (27 g) as a solid.

### Preparation Example 34

N-(6-Chloropyridin-2-yl)-2,2-dimethylpropanamide (13 g) was dissolved in THF (100 mL), then n-butyllithium (1.6 M in n-hexane, 100 mL) was added dropwise to the solution at −50 degrees Celsius under an argon atmosphere, and the mixture was stirred under ice brine cooling for 2 hours. A mixture of N-methoxy-N-methylacetamide (13 mL) and THF (20 mL) was added dropwise to the mixture at −40 degrees Celsius, and the mixture was stirred at the same temperature for 1.5 hours. A saturated aqueous ammonium chloride solution was added to the mixture, then the mixture was extracted with ethyl acetate and the organic layer was dried over anhydrous magnesium sulfate. Any insoluble material was filtered off, the filtrate was concentrated under reduced pressure, and the obtained residue was purified by silica gel column chromatography (hexane/ethyl acetate) to obtain N-(3-acetyl-6-chloropyridin-2-yl)-2,2-dimethylpropanamide (12 g) as a solid.

### Preparation Example 35

A mixture of 7-chloro-2-{2,6-difluoro-4-[(3S)-3-fluoropyrrolidine-1-sulfonyl]phenyl}-4-methyl-1,8-naphthyridine (150 mg), tris(dibenzylideneacetone)dipalladium (0) (32 mg), 5-(di-tert-butylphosphinyl)-1′,3′,5′-triphenyl-1′H-1,4′-bipyrazole (35 mg), and toluene (3 mL) was stirred at 90 degrees Celsius for 5 minutes under an argon atmosphere. To the mixture was added tert-butyl-2-hydroxyacetate (140 microliters) and cesium carbonate (340 mg), and the mixture was stirred at 90 degrees Celsius for 3 hours under an argon atmosphere. After the mixture was allowed to cool to room temperature, ethyl acetate and water were added thereto to perform extraction, and the organic layer was dried over anhydrous magnesium sulfate. Any insoluble material was filtered off, the filtrate was concentrated under reduced pressure, and the obtained residue was purified by silica gel column chromatography (hexane/ethyl acetate: using neutral silica gel, hexane/ethyl acetate: using basic silica gel) to obtain tert-butyl [(7-{2,6-difluoro-4-[(3S)-3-fluoropyrrolidine-1-sulfonyl]phenyl}-5-methyl-1,8-naphthyridin-2-yl)oxy]acetate (120 mg) as a solid.

### Preparation Example 36

A mixture of 7-chloro-2-{2,6-difluoro-4-[(3S)-3-fluoropyrrolidine-1-sulfonyl]phenyl}-4-methyl-1,8-naphthyridine (200 mg), tert-butyl 4-(4,4,5,5-tetramethyl-1,3,2-dioxaborolan-2-yl)-3,6-dihydropyridine-1 (2H)-carboxylate (290 mg), SPhos Pd G4 (18 mg, CAS number: 1599466-87-1, Sigma-Aldrich), cesium fluoride (210 mg), and 1,4-dioxane (3 mL) was stirred at 100 degrees Celsius for 3 hours under microwave irradiation. Ethyl acetate and water were added thereto to perform extraction, and the organic layer was dried over anhydrous magnesium sulfate. Any insoluble material was filtered off, the filtrate was concentrated under reduced pressure, and the obtained residue was purified by silica gel column chromatography (hexane/ethyl acetate: using neutral silica gel, hexane/ethyl acetate: using basic silica gel) to obtain tert-butyl 4-(7-{2,6-difluoro-4-[(3S)-3-fluoropyrrolidine-1-sulfonyl]phenyl}-5-methyl-1,8-naphthyridin-2-yl)-3,6-dihydropyridine-1 (2H)-carboxylate (170 mg) as an oil.

### Preparation Example 37

7-Bromo-2-chloro-5-methylquinoline (100 mg) was dissolved in toluene (4 mL), then tert-butyl piperazine-1-carboxylate (150 mg) was added and the mixture was stirred at 140 degrees Celsius for 2 hours under microwave irradiation. tert-Butyl piperazine-1-carboxylate (150 mg) was added to the mixture and the mixture was stirred at 140 degrees Celsius for 2 hours under microwave irradiation. The mixture was allowed to cool to room temperature and then purified by silica gel column chromatography (hexane/ethyl acetate) to obtain tert-butyl 4-(7-bromo-5-methylquinolin-2-yl) piperazine-1-carboxylate (31 mg) as an oil.

### Preparation Example 38

7-Bromo-2-iodo-5-methylquinoline (100 mg) was dissolved in 1,4-dioxane (3 mL), then copper (I) cyanide (35 mg), tris(dibenzylideneacetone)dipalladium (0) (15 mg), and 1,1′-bis(diphenylphosphino)ferrocene (9 mg) were added to the mixture and stirred at 120 degrees Celsius for 4 hours under microwave irradiation. The mixture was allowed to cool to room temperature and then purified by silica gel column chromatography (hexane/ethyl acetate) to obtain 7-bromo-5-methylquinoline-2-carbonitrile (33 mg) as a solid.

### Preparation Example 39

N,N-diethylaniline (0.1 mL) was added to a mixture of 7-bromo-8-fluoro-5-methylquinolin-2 (1H)-one (300 mg) and phosphoryl chloride (1.3 mL) at room temperature and stirred at 100 degrees Celsius overnight under an argon atmosphere. The mixture was allowed to cool to room temperature and poured into ice water, and the mixture was stirred at room temperature for 1 hour. The resulting mixture was extracted with chloroform and the organic layer was washed with brine then dried over anhydrous sodium sulfate. Any insoluble material was filtered off, and the filtrate was concentrated under reduced pressure. The obtained residue was purified by silica gel column chromatography (hexane/ethyl acetate) to obtain 7-bromo-2-chloro-8-fluoro-5-methylquinoline (300 mg) as a solid.

### Preparation Example 40

Sodium iodide (1.6 g) and acetyl chloride (0.23 mL) were added to a mixture of 7-bromo-2-chloro-8-fluoro-5-methylquinoline (290 mg) and acetonitrile (7 mL) at room temperature and stirred at 80 degrees Celsius for 6 hours under an argon atmosphere. The mixture was allowed to cool to room temperature and then concentrated under reduced pressure. A saturated aqueous sodium hydrogen carbonate solution was added to the obtained residue, then the mixture was extracted with chloroform and the organic layer was washed with sodium thiosulfate solution, water and brine, then dried over anhydrous sodium sulfate. Any insoluble material was filtered off and the filtrate was concentrated under reduced pressure to obtain 7-bromo-8-fluoro-2-iodo-5-methylquinoline (400 mg) as a solid.

### Preparation Example 41

Methyl 2-[4-(benzylsulfanyl)-2,6-difluorophenyl]-4-methylquinoline-7-carboxylate (440 mg) was dissolved in acetic acid (7 mL) and water (1.8 mL), then N-chlorosuccinimide (810 mg) was added under ice-cooling, and the mixture was stirred at room temperature for 3 hours. Cold water was added to the reaction mixture under ice-cooling, and then the solid was collected by filtration. After dissolving the solid in chloroform, cold water was added to perform extraction, and the organic layer was dried over anhydrous sodium sulfate. Any insoluble material was filtered off, and the filtrate was concentrated. The obtained residue (200 mg) was dissolved in dichloromethane (2 mL), then (3S)-3-fluoropyrrolidine monohydrochloride (91 mg) and N,N-diisopropylethylamine (0.17 mL) were added, and the mixture was stirred at room temperature for 0.5 hours. Dichloromethane and water were added to the reaction mixture, and the mixture was stirred at room temperature for 5 minutes and extraction was performed. The organic layer was dried over anhydrous sodium sulfate. Any insoluble material was filtered off, and the filtrate was concentrated. The obtained residue was purified by silica gel column chromatography (hexane/ethyl acetate) to obtain methyl 2-{2,6-difluoro-4-[(3S)-3-fluoropyrrolidine-1-sulfonyl]phenyl}-4-methylquinoline-7-carboxylate (90 mg) as a solid.

### Preparation Example 42

Methyl 2-[4-(benzylsulfanyl)-2,6-difluorophenyl]-3-fluoro-4-methylquinoline-7-carboxylate (500 mg) was suspended in acetic acid (8 mL) and water (2 mL). N-Chlorosuccinimide (590 mg) was added to the mixture under ice-cooling, and the mixture was stirred at room temperature for 1 hour. To the mixture was added ice at room temperature and stirred until the ice was dissolved, and then the solid was collected by filtration. The solid was dissolved in chloroform to perform extraction, and the organic layer was dried over anhydrous magnesium sulfate, concentrated under reduced pressure. Dichloromethane (12 mL) and triethylamine (0.46 mL) were added to the obtained residue, then 4-fluoropiperidine monohydrochloride (170 mg) was added thereto, and the mixture was stirred at room temperature for 15 minutes. Chloroform and water were added to the mixture to perform extraction, and the organic layer was concentrated under reduced pressure. The obtained residue was purified by silica gel column chromatography (hexane/ethyl acetate) to obtain methyl 2-[2,6-difluoro-4-(4-fluoropiperidine-1-sulfonyl)phenyl]-3-fluoro-4-methylquinoline-7-carboxylate (380 mg) as a solid.

### Preparation Example 110

Methyl 2-{4-[(3S)-3-fluoropyrrolidine-1-sulfonyl]-2-(hydroxymethyl)phenyl}-4-methylquinoline-7-carboxylate (90 mg) was dissolved in DMF (2 mL), then sodium hydride (60% oily, 16 mg) and iodomethane (30 microliters) were added thereto under ice-cooling, and the mixture was stirred at room temperature for 4 hours. After adding water to the mixture, ethyl acetate was added thereto to perform extraction, and the organic layer was dried over anhydrous magnesium sulfate. The insoluble material was filtered off, the filtrate was concentrated under reduced pressure. The obtained residue was purified by silica gel column chromatography (hexane/ethyl acetate) to obtain methyl 2-{4-[(3S)-3-fluoropyrrolidine-1-sulfonyl]-2-(methoxymethyl)phenyl}-4-methylquinoline-7-carboxylate (110 mg) as a solid.

### Preparation Example 120

A mixture of (2E)-N-(3-bromo-2-fluoro-5-methylphenyl)-3-ethoxyprop-2-enamide (700 mg) and concentrated sulfuric acid (6 mL) was stirred at room temperature overnight under an argon atmosphere. The reaction mixture was poured into ice, and the mixture was stirred at room temperature for 1 hour. The solid was collected by filtration, washed with water and dried under reduced pressure. The obtained solid was suspended in ethyl acetate. The solid was collected by filtration, washed with ethyl acetate and dried to obtain 7-bromo-8-fluoro-5-methylquinolin-2 (1H)-one (550 mg) as a solid.

### Preparation Example 123

To a mixture of diethyl (2,2-diethoxyethyl) propanedioate (0.47 g) and THF (5 mL) was added sodium hydride (60% oily, 82 mg) under ice-cooling in an argon atmosphere and the mixture was stirred at room temperature for 1 hour. A solution of 1-bromo-3-(bromomethyl)-5-methoxybenzene (0.50 g) in THF (3 mL) was added, and the mixture was stirred at room temperature for 4 hours. Methanol (3 mL) and 3 M aqueous potassium hydroxide solution (3 mL) were added, and the mixture was stirred at 120 degrees Celsius for 1 hour under microwave irradiation. The mixture was concentrated under reduced pressure. To the obtained residue was added DMF (3 mL) and iodomethane (0.3 mL) and the mixture was stirred at room temperature overnight. To the mixture was added 1 M hydrochloric acid (9 mL) under ice-cooling, then the mixture was extracted with ethyl acetate and the organic layer was washed with brine, and dried over anhydrous magnesium sulfate. Any insoluble material was filtered off, the filtrate was concentrated under reduced pressure. The obtained residue was purified by silica gel column chromatography (hexane/ethyl acetate) to obtain methyl 2-[(3-bromo-5-methoxyphenyl)methyl]-4,4-diethoxybutanoate (0.48 g) as an oil.

### Preparation Example 124

To a mixture of 4,5-dichloro-3,6-dioxocyclohexa-1,4-diene-1,2-dicarbonitrile (0.27 g) and 80% sulfuric acid (3.9 mL) was added methyl 2-[(3-bromo-5-methoxyphenyl)methyl]-4,4-diethoxybutanoate (0.39 g) in methanol (3.9 mL) under ice-cooling, and the mixture was stirred at room temperature for 0.5 hours. After adding water to the mixture under ice-cooling, the solid was collected by filtration. The solid was dissolved in ethyl acetate, then the organic layer was washed with water, brine, and dried over anhydrous sodium sulfate. Any insoluble material was filtered off, the filtrate was concentrated under reduced pressure. The obtained residue was purified by silica gel column chromatography (hexane/ethyl acetate) to obtain methyl 5-bromo-7-methoxynaphthalene-2-carboxylate (0.12 g) as a solid.

### Preparation Example 125

A mixture of methyl 5-bromo-7-methoxynaphthalene-2-carboxylate (0.12 g), 2,4,6-trimethyl-1,3,5,2,4,6-trioxatriborinane (180 microliters), SPhos Pd G4 (31 mg, CAS number: 1599466-87-1, Sigma-Aldrich), cesium carbonate (0.38 g), 1,4-dioxane (4 mL), and water (0.4 mL) was stirred at 100 degrees Celsius for 0.5 hours under microwave irradiation. Ethyl acetate was added, and the mixture was filtered through Celite and the filtrate was concentrated under reduced pressure. The obtained residue was purified by silica gel column chromatography (hexane/ethyl acetate) to obtain methyl 7-methoxy-5-methylnaphthalene-2-carboxylate (80 mg) as a solid.

### Preparation Example 126

To a mixture of methyl 7-methoxy-5-methylnaphthalene-2-carboxylate (43 mg) and dichloromethane (1.3 mL) was added tribromoborane (1 M in dichloromethane, 0.51 mL) under ice-cooling in an argon atmosphere, and the mixture was stirred at room temperature for 0.5 hours. Methanol was added to the mixture under ice-cooling, then the mixture was concentrated under reduced pressure. The obtained residue was purified by silica gel column chromatography (hexane/ethyl acetate) to obtain methyl 7-hydroxy-5-methylnaphthalene-2-carboxylate (27 mg) as a solid.

### Preparation Example 127

A mixture of methyl 5-methyl-7-[(trifluoromethanesulfonyl)oxy]naphthalene-2-carboxylate (35 mg), 4,4,4′,4′,5,5,5′,5′-octamethyl-2,2′-bi-1,3,2-dioxaborolane (35 mg), [1,1′-bis(diphenylphosphino)ferrocene]dichloropalladium(II) dichloromethane adduct (17 mg), potassium acetate (30 mg), and 1,4-dioxane (1 mL) was stirred at 100 degrees Celsius for 3 hours under an argon atmosphere. After the mixture was allowed to cool to room temperature, (3S)-1-(4-bromo-3,5-difluorobenzene-1-sulfonyl)-3-fluoropyrrolidine (56 mg), potassium carbonate (40 mg), and water (0.2 mL) were added and stirred at 100 degrees Celsius overnight under an argon atmosphere. After the mixture was allowed to cool to room temperature, the mixture was added chloroform, filtered through Celite and washed with chloroform. The filtrate was concentrated under reduced pressure, and the obtained residue was purified by silica gel column chromatography (hexane/ethyl acetate) to obtain methyl 7-{2,6-difluoro-4-[(3S)-3-fluoropyrrolidine-1-sulfonyl]phenyl}-5-methylnaphthalene-2-carboxylate (34 mg) as a solid.

### Example 1

Methyl 2-{2,6-difluoro-4-[(3S)-3-fluoropyrrolidine-1-sulfonyl]phenyl}-4-methylquinoline-7-carboxylate (90 mg) was dissolved in THF (1 mL) and ethanol (1 mL), then 1 M aqueous sodium hydroxide solution (1 mL) was added and the mixture was stirred at room temperature for 1 hour. After adding 1 M hydrochloric acid (1 mL) to the mixture, chloroform and brine were added thereto to perform extraction, and the organic layer was dried over anhydrous sodium sulfate. Any insoluble material was filtered off and the filtrate was concentrated, then diethyl ether (2 mL) was added to the residue. The obtained solid was collected by filtration and dried under reduced pressure to obtain 2-{2,6-difluoro-4-[(3S)-3-fluoropyrrolidine-1-sulfonyl]phenyl}-4-methylquinoline-7-carboxylic acid (80 mg) as a solid.

### Example 2

2-{2,6-Difluoro-4-[(3S)-3-fluoropyrrolidine-1-sulfonyl]phenyl}-4-methylquinoline-7-carboxylic acid (3.0 g) was dissolved in THF (61 mL), then 1 M aqueous sodium hydroxide solution (6.8 mL) was added to the mixture and stirred at room temperature for 2 hours. The reaction mixture was concentrated under reduced pressure, then ethanol was added and the mixture was concentrated again. Ethanol (30 mL) was added to the mixture and the mixture was stirred under ice-cooling. The solid was collected by filtration, and dried under reduced pressure to obtain sodium 2-{2,6-difluoro-4-[(3S)-3-fluoropyrrolidine-1-sulfonyl]phenyl}-4-methylquinoline-7-carboxylate (2.7 g) as a solid.

### Example 3

A mixture of 2-[2,6-difluoro-4-(4-fluoropiperidine-1-sulfonyl)phenyl]-4-methylquinoline-7-carboxylic acid (290 mg), 2-methyl-2,6-diazaspiro[3.3]heptane dihydrochloride (72 mg), N,N-diisopropylethylamine (0.3 mL), HATU (150 mg), and dichloromethane (3 mL) was stirred at room temperature overnight. The mixture was purified by silica gel column chromatography (hexane/ethyl acetate: using basic silica gel, chloroform/methanol: using neutral silica gel) to obtain {2-[2,6-difluoro-4-(4-fluoropiperidine-1-sulfonyl)phenyl]-4-methylquinolin-7-yl}(6-m ethyl-2,6-diazaspiro[3.3]heptan-2-yl) methanone (170 mg) as a solid.

### Example 4

A mixture of 2-[2,6-difluoro-4-(4-fluoropiperidine-1-sulfonyl)phenyl]-4-methylquinoline-7-carboxylic acid (200 mg), tert-butyl (azetidin-3-yl) carbamate monohydrochloride (140 mg), N,N-diisopropylethylamine (0.35 mL), HATU (340 mg), and dichloromethane (6 mL) was stirred at room temperature overnight. A saturated aqueous sodium hydrogen carbonate solution and water were added to the reaction mixture. The mixture was extracted with dichloromethane, and the organic layer was concentrated under reduced pressure. The obtained residue was purified by silica gel column chromatography (hexane/ethyl acetate). The resulting product was dissolved in dichloromethane (6 mL), then trifluoroacetic acid (1 mL) was added and the mixture was stirred at room temperature for 1 hour. The mixture was concentrated under reduced pressure, then a saturated aqueous sodium hydrogen carbonate solution and water were added and the mixture was extracted with chloroform. The organic layer was concentrated under reduced pressure, and the obtained residue was purified by silica gel column chromatography (chloroform/methanol) to obtain (3-aminoazetidin-1-yl) {2-[2,6-difluoro-4-(4-fluoropiperidine-1-sulfonyl)phenyl]-4-methylquinolin-7-yl}methanone (160 mg) as a solid.

### Example 5

Methyl 2-[2-fluoro-4-(4-fluoropiperidine-1-sulfonyl)phenyl]-4-methylquinoline-7-carboxylate (400 mg) was suspended in methanol (4 mL) and THF (4 mL), then 1 M aqueous sodium hydroxide solution (1.7 mL) was added at room temperature, and the mixture was stirred at 75 degrees Celsius for 1 hour. After the reaction mixture was allowed to cool to room temperature, 1 M hydrochloric acid was added to the mixture, the reaction mixture was concentrated under reduced pressure, and water and chloroform were added to perform extraction. The organic layer was concentrated under reduced pressure to obtain 2-[2-fluoro-4-(4-fluoropiperidine-1-sulfonyl)phenyl]-4-methylquinoline-7-carboxylic acid (370 mg) as a solid.

### Example 6

tert-Butyl [(7-{2,6-difluoro-4-[(3S)-3-fluoropyrrolidine-1-sulfonyl]phenyl}-5-methyl-1,8-naphthyridin-2-yl)oxy]acetate (120 mg) was dissolved in methanol (1 mL), then hydrogen chloride (4 M in 1,4-dioxane, 1 mL) was added and the mixture was stirred at room temperature overnight. The mixture was concentrated under reduced pressure. The resulting residue was dissolved in methanol (1 mL), then 1 M aqueous sodium hydroxide solution (1 mL) was added and the mixture was stirred at room temperature for 1 hour. To the mixture was added 1 M hydrochloric acid to perform neutralization, and the mixture was concentrated under reduced pressure. The obtained residue was purified by silica gel column chromatography (chloroform/methanol) to obtain [(7-{2,6-difluoro-4-[(3S)-3-fluoropyrrolidine-1-sulfonyl]phenyl}-5-methyl-1,8-naphthyridin-2-yl)oxy]acetic acid (85 mg) as a solid.

### Example 7

The reaction was conducted in two batches. To a mixture of 7-bromo-8-fluoro-2-{2-fluoro-4-[(3S)-3-fluoropyrrolidine-1-sulfonyl]phenyl}-4-methylquinoline (280 mg), potassium carbonate (160 mg) and dicyclohexyl (3-dicyclohexylphosphaniumylpropyl)phosphonium; ditetrafluoroborate (69 mg) in DMF (5.5 mL) was added palladium acetate (13 mg). The mixture was degassed and purged with CO three times and then stirred at 115 degrees Celsius under CO atmosphere for 12 hours. The mixture was filtered, and the filtrate was concentrated under reduced pressure. The crude product was purified by Biotage flash reversed-phase C-18 column chromatography (methanol/water, 0.1% acetic acid condition) to obtain 8-fluoro-2-{2-fluoro-4-[(3S)-3-fluoropyrrolidine-1-sulfonyl]phenyl}-4-methylquinoline-7-carboxylic acid (70 mg) as a solid.

### Example 8

7-{2,6-Difluoro-4-[(3S)-3-fluoropyrrolidine-1-sulfonyl]phenyl}-5-methylquinoline-2-carbonitrile (20 mg) was suspended in methanol (1 mL), then sodium methoxide (28% in methanol, 0.03 mL) was added and the mixture was stirred at room temperature for 3 hours. Sodium methoxide (28% in methanol, 0.02 mL) was further added to the mixture, and the mixture was stirred at room temperature overnight. To the mixture was added 1 M hydrochloric acid (0.11 mL) under ice-cooling, and the mixture was stirred at room temperature for 30 minutes. A saturated aqueous sodium hydrogen carbonate solution was added to the mixture, then the mixture was extracted with chloroform and the organic layer was washed with brine, and then dried over anhydrous sodium sulfate. Any insoluble material was filtered off, the filtrate was concentrated under reduced pressure. The obtained residue was purified by silica gel column chromatography (hexane/ethyl acetate). The obtained residue was dissolved in THF (0.3 mL) and methanol (0.3 mL), then water (0.3 mL) and lithium hydroxide (3 mg) were added and the mixture was stirred at room temperature for 4 hours. To the mixture was added 1 M aqueous sodium hydroxide solution (0.1 mL) and stirred at room temperature for 3 hours and then stirred at 50 degrees Celsius overnight. The mixture was neutralized with 1 M hydrochloric acid and then extracted with chloroform/2-propanol. The organic layer was washed with brine, then dried over anhydrous sodium sulfate. Any insoluble material was filtered off, and the filtrate was concentrated under reduced pressure. Ethyl acetate and hexane were added to the obtained residue, and the solid was collected by filtration, and dried under reduced pressure to obtain 7-{2-fluoro-4-[(3S)-3-fluoropyrrolidine-1-sulfonyl]-6-methoxyphenyl}-5-methylquinoline-2-carboxylic acid (4.4 mg) as a solid.

### Example 9

2-{2-Fluoro-4-[(3S)-3-fluoropyrrolidine-1-sulfonyl]phenyl}-4-methylquinoline-7-carboxylic acid (30 mg) and cyclopropanesulfonamide (17 mg) were suspended in dichloromethane (3 mL), then 4-dimethylaminopyridine (10 mg) and 1-ethyl-3-(3-dimethylaminopropyl) carbodiimide hydrochloride (21 mg) were added and the mixture was stirred at room temperature for 3 hours. Chloroform and water were added to the mixture to perform extraction, and the organic layer was dried over anhydrous sodium sulfate. Any insoluble material was filtered off, and the filtrate was concentrated under reduced pressure. The obtained residue was purified by silica gel column chromatography (chloroform/methanol) to obtain N-(cyclopropanesulfonyl)-2-{2-fluoro-4-[(3S)-3-fluoropyrrolidine-1-sulfonyl]phenyl}-4-methylquinoline-7-carboxamide (32 mg) as a solid.

### Example 10

A mixture of 2-{2,6-difluoro-4-[(3S)-3-fluoropyrrolidine-1-sulfonyl]phenyl}-4-methylquinoline-7-carboxylic acid (50 mg), N-(tert-butoxycarbonyl)-N-methyl-1,2-ethylenediamine (40 mg), N,N-diisopropylethylamine (0.06 mL), HATU (64 mg), and dichloromethane (1 mL) was stirred at room temperature for 1 hour. The mixture was purified by silica gel column chromatography (hexane/ethyl acetate). The obtained product (72 mg) was dissolved in ethyl acetate (1 mL), then hydrogen chloride (4 M in ethyl acetate, 1 mL) was added and the mixture was stirred at room temperature overnight. The mixture was concentrated under reduced pressure, then ethyl acetate was added and the mixture was triturated. The solid was collected by filtration and dried under reduced pressure to obtain 2-{2,6-difluoro-4-[(3S)-3-fluoropyrrolidine-1-sulfonyl]phenyl}-4-methyl-N-[2-(methyl amino)ethyl]quinoline-7-carboxamide hydrochloride (55 mg) as a solid.

### Example 11

To a mixture of 2-{2,6-difluoro-4-[(3S)-3-fluoropyrrolidine-1-sulfonyl]phenyl}-3-fluoro-4-methylquinoline-7-carboxylic acid (60 mg), ethyl 3-methylazetidine-3-carboxylate hydrochloride (31 mg), and dichloromethane (2 mL) was added N,N-diisopropylethylamine (67 mg) and HATU (98 mg), and the mixture was stirred at room temperature for 12 hours. DMF (1 mL) and N,N-diisopropylethylamine (10 drops) were added to the mixture, and the mixture was stirred at room temperature for 5 hours. A saturated aqueous sodium hydrogen carbonate solution and water were added to the reaction mixture. The mixture was extracted with ethyl acetate and the organic layer was washed with brine, then dried over anhydrous magnesium sulfate. Any insoluble material was filtered off, the filtrate was concentrated under reduced pressure. The obtained residue was dissolved in THF (1.5 mL) and methanol (1.5 mL), then 1 M aqueous sodium hydroxide solution (0.75 mL) was added and the mixture was stirred at room temperature for 1.5 hours. To the mixture was added 1 M hydrochloric acid (2 mL), the mixture was concentrated under reduced pressure. The obtained residue was purified by silica gel column chromatography (water/acetonitrile: using ODS silica gel) to obtain the crude product. The crude 1-(2-{2,6-difluoro-4-[(3S)-3-fluoropyrrolidine-1-sulfonyl]phenyl}-3-fluoro-4-methylquinoline-7-carbonyl)-3-methylazetidine-3-carboxylic acid (55 mg) was added to diisopropyl ether (2 mL) and hexane (3 mL), and the solid was collected by filtration, washed with hexane, and dried under reduced pressure to obtain 1-(2-{2,6-difluoro-4-[(3S)-3-fluoropyrrolidine-1-sulfonyl]phenyl}-3-fluoro-4-methylquinoline-7-carbonyl)-3-methylazetidine-3-carboxylic acid (36 mg) as a solid.

### Example 12

To a mixture of 2-{2,6-difluoro-4-[(3S)-3-fluoropyrrolidine-1-sulfonyl]phenyl}-4-methylquinoline-7-carboxylic acid (60 mg), methyl (2S)-2-amino-3-[(tert-butoxycarbonyl)amino]propanoate monohydrochloride (69 mg), and dichloromethane (2 mL) was added N,N-diisopropylethylamine (140 microliters) and HATU (100 mg) and stirred at room temperature for 17 hours. To the mixture was added a saturated aqueous sodium hydrogen carbonate solution and water, then the mixture was extracted with chloroform and the organic layer was concentrated under reduced pressure. The obtained residue was dissolved in THF (1.5 mL) and methanol (1.5 mL), and then 1 M aqueous sodium hydroxide solution (0.75 mL) was added and the mixture was stirred at room temperature for 1.5 hours. To the mixture was added 1 M hydrochloric acid (2 mL) and the mixture was concentrated under reduced pressure, and the obtained residue was purified by silica gel column chromatography (water/acetonitrile: using ODS silica gel). The resulting product (38 mg) was dissolved in THF (2 mL), then hydrogen chloride (4 M in 1,4-dioxane, 8 mL) was added, and the mixture was stirred at room temperature for 1 hour. The mixture was concentrated under reduced pressure, and the obtained residue was purified by silica gel column chromatography (water/acetonitrile: using ODS silica gel) to obtain the crude product. The obtained crude (2S)-3-amino-2-(2-{2,6-difluoro-4-[(3S)-3-fluoropyrrolidine-1-sulfonyl]phenyl}-4-methylquinoline-7-carboxamido) propanoic acid hydrochloride (30 mg) was added to diisopropyl ether (3 mL) and the solid was collected by filtration, and dried under reduced pressure to obtain (2S)-3-amino-2-(2-{2,6-difluoro-4-[(3S)-3-fluoropyrrolidine-1-sulfonyl]phenyl}-4-methylquinoline-7-carboxamido) propanoic acid hydrochloride (26 mg) as a solid.

### Example 13

tert-Butyl {2-[(2-{2-fluoro-4-[(3S)-3-fluoropyrrolidine-1-sulfonyl]phenyl}-4-methylquinolin-7-yl)oxy]ethyl}carbamate (5.7 g) was dissolved in chloroform (40 mL) and methanol (20 mL), then hydrogen chloride (4 M in 1,4-dioxane, 20 mL) was added and the mixture was stirred at room temperature for 5 hours. After concentrating the reaction mixture under reduced pressure, ethyl acetate was added to the residue, and the solid was collected by filtration and dried under reduced pressure to obtain 2-[(2-{2-fluoro-4-[(3S)-3-fluoropyrrolidine-1-sulfonyl]phenyl}-4-methylquinolin-7-yl)oxy]ethan-1-amine hydrochloride (5.0 g) as a solid.

### Example 14

To a mixture of tert-butyl 6-{2-[2,6-difluoro-4-(4-fluoropiperidine-1-sulfonyl) phenyl]-4-methylquinoline-7-carbonyl}-2,6-diazaspiro[3.3]heptane-2-carboxylate (150 mg) and dichloromethane (3 mL) was added trifluoroacetic acid (3 mL) and stirred at room temperature for 0.5 hours. The mixture was concentrated, then chloroform and 1 M aqueous sodium hydroxide solution were added. The aqueous layer was extracted with chloroform and the organic layer was concentrated. Dichloromethane and hexane were added to the obtained residue, the solid was collected by filtration and washed with hexane to obtain (2,6-diazaspiro[3.3]heptan-2-yl) {2-[2,6-difluoro-4-(4-fluoropiperidine-1-sulfonyl)phenyl]-4-methylquinolin-7-yl}methanone (68 mg) as a solid.

### Example 15

To a mixture of tert-butyl [2-(7-{2,6-difluoro-4-[(3S)-3-fluoropyrrolidine-1-sulfonyl]phenyl}-8-fluoro-5-methylquinoline-2-carboxamido)ethyl](methyl)carbamate (11 mg) and dichloromethane (1 mL) was added trifluoroacetic acid (0.05 mL), and the mixture was stirred at room temperature for 2 hours under an argon atmosphere. The mixture was purified by silica gel column chromatography (chloroform/methanol: using basic silica gel) and concentrated. Ethyl acetate (0.5 mL) was added to the obtained residue, then hydrogen chloride (4 M in ethyl acetate, 0.01 mL) was added and the mixture was concentrated under reduced pressure. To the obtained residue was added 2-propanol and toluene, and the mixture was triturated and concentrated under reduced pressure. The obtained residue was triturated with dichloromethane and hexane, concentrated under reduced pressure, and then dried under reduced pressure to obtain 7-{2,6-difluoro-4-[(3S)-3-fluoropyrrolidine-1-sulfonyl]phenyl}-8-fluoro-5-methyl-N-[2-(methylamino)ethyl]quinoline-2-carboxamide hydrochloride (6.6 mg) as a solid.

### Example 16

A mixture of 3-(2-{2,6-difluoro-4-[(3S)-3-fluoropyrrolidine-1-sulfonyl]phenyl}-3-fluoro-4-methylquinolin-7-yl) propanal (60 mg), methylamine (2 M in THF, 0.62 mL), and dichloromethane (1.5 mL) was stirred at room temperature for 10 minutes. Sodium triacetoxyborohydride (50 mg) was added to the mixture under ice-cooling, and the mixture was warmed over 2 hours to room temperature and then stirred at room temperature for 1 hour. A saturated aqueous sodium hydrogen carbonate solution was added to the mixture, then the mixture was extracted with chloroform, and the organic layer was dried over anhydrous sodium sulfate, concentrated under reduced pressure, and the obtained residue was purified by silica gel column chromatography (chloroform/methanol: using basic silica gel) to obtain the crude product. To a mixture of the resulting crude 3-(2-{2,6-difluoro-4-[(3S)-3-fluoropyrrolidine-1-sulfonyl]phenyl}-3-fluoro-4-methylquinolin-7-yl)-N-methylpropan-1-amine (38 mg) and ethyl acetate (3 mL) was added hydrogen chloride (4 M in ethyl acetate, 0.038 mL), and the mixture was stirred at room temperature for 15 minutes. The solid was collected by filtration and dried under reduced pressure to obtain 3-(2-{2,6-difluoro-4-[(3S)-3-fluoropyrrolidine-1-sulfonyl]phenyl}-3-fluoro-4-methylquinolin-7-yl)-N-methylpropan-1-amine hydrochloride (34 mg) as a solid.

### Example 17

A mixture of 2-{2,6-difluoro-4-[(3S)-3-fluoropyrrolidine-1-sulfonyl]phenyl}-4-methyl-7-(piperidin-4-yl)-1,8-naphthyridine hydrochloride (35 mg), formaldehyde (36% aqueous solution, 0.05 mL), sodium triacetoxyborohydride (26 mg), acetic acid (0.01 mL), dichloromethane (2 mL), and methanol (0.2 mL) was stirred at room temperature for 30 minutes. A saturated aqueous sodium hydrogen carbonate solution and chloroform were added to the mixture to perform extraction, and the organic layer was concentrated under reduced pressure. The obtained residue was purified by silica gel column chromatography (chloroform: using basic silica gel). The obtained solid was dissolved in methanol (1 mL), then hydrogen chloride (4 M in 1,4-dioxane, 0.2 mL) was added and the mixture was concentrated under reduced pressure to obtain 2-{2,6-difluoro-4-[(3S)-3-fluoropyrrolidine-1-sulfonyl]phenyl}-4-methyl-7-(1-methylpiperidin-4-yl)-1,8-naphthyridine hydrochloride (30 mg) as a solid.

### Example 18

To a mixture of methyl 2-[2,6-difluoro-4-(4-fluoropiperidine-1-sulfonyl)phenyl]-3-fluoro-4-methylquinoline-7-carboxylate (0.38 g) and THF (4 mL) was added 1 M aqueous sodium hydroxide solution (2.5 mL) under ice-cooling and the mixture was stirred at room temperature for 3 hours. The mixture was concentrated under reduced pressure, then 1 M hydrochloric acid (2.5 mL) was added to the residue under ice-cooling and stirred at room temperature for 0.5 hours. The solid was collected by filtration and dried under reduced pressure to obtain 2-[2,6-difluoro-4-(4-fluoropiperidine-1-sulfonyl)phenyl]-3-fluoro-4-methylquinoline-7-carboxylic acid (0.33 g) as a solid.

### Example 19

Methyl 2-[2,6-difluoro-4-(4-fluoropiperidine-1-sulfonyl)phenyl]-4-methylquinoline-7-carboxy late (8.4 g) was suspended in THF (150 mL), and 1 M aqueous sodium oxide solution (50 mL) was added and the mixture was stirred at room temperature overnight. The mixture was ice-cooled, then 1 M hydrochloric acid was added to perform neutralization, and the mixture was concentrated under reduced pressure to remove most of THF, then stirred overnight. The solid was collected by filtration and washed with water, then the obtained solid was washed with chloroform. The solid was collected by filtration and dried under reduced pressure to obtain 2-[2,6-difluoro-4-(4-fluoropiperidine-1-sulfonyl)phenyl]-4-methylquinoline-7-carboxylic acid (4.8 g) as a solid. Further, the filtrate was concentrated under reduced pressure, and the obtained residue was purified by silica gel column chromatography (chloroform/methanol) to obtain 2-[2,6-difluoro-4-(4-fluoropiperidine-1-sulfonyl)phenyl]-4-methylquinoline-7-carboxylic acid (0.7 g) as a solid. The resulting solids of 2-[2,6-difluoro-4-(4-fluoropiperidine-1-sulfonyl)phenyl]-4-methylquinoline-7-carboxylic acid (4.8 g and 0.7 g) were mixed, and chloroform, methanol and acetone were added to dissolve the solid, then activated carbon was added thereto, and the mixture was stirred for 15 minutes then filtered through Celite. The filtrate was concentrated under reduced pressure. Acetone (160 mL) was added to the obtained solid and the mixture was stirred, then water (18 mL) was added and the mixture was stirred at 65 degrees Celsius for 2 hours. After the mixture was allowed to cool to room temperature, water (140 mL) was added over 30 minutes, and the mixture was stirred at room temperature for 30 minutes and then further stirred under ice-cooling for 1 hour. The resulting solid was collected by filtration, washed with ice-cooled acetone and water (1:1) and then dried under reduced pressure to obtain 2-[2,6-difluoro-4-(4-fluoropiperidine-1-sulfonyl)phenyl]-4-methylquinoline-7-carboxylic acid (4.8 g) as a solid.

### Example 20

Methyl 2-{2,6-difluoro-4-[(3S)-3-fluoropyrrolidine-1-sulfonyl]phenyl}-3-fluoro-4-methylquinoline-7-carboxylate (200 mg) was suspended in THF (3 mL) and ethanol (3 mL), then 1 M aqueous sodium hydroxide solution (2 mL) was added and the mixture was stirred at room temperature for 1 hour. After adding 1 M hydrochloric acid (2 mL) to the reaction mixture, water (20 mL) was added, and the mixture was stirred under ice-cooling for 5 minutes. The solid was collected by filtration, dried under reduced pressure to obtain 2-{2,6-difluoro-4-[(3S)-3-fluoropyrrolidine-1-sulfonyl]phenyl}-3-fluoro-4-methylquinoline-7-carboxylic acid (170 mg) as a solid.

The compounds of Preparation Examples and Examples shown in the tables below were produced in the same manner as in the above Preparation Examples or Examples. In Tables 3 to 28 and 35 to 51, “y HCl” means the compound is free form or mono hydrochloride, and “m HCl” means the compound is free form, monohydrochloride or dihydrochloride. Compounds marked with “*” in Tables 3 to 28 and 35 to 51 are racemic mixture.

TABLE 3

| PEx | Str |
|---|---|
| 1 | 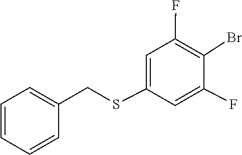 [SMILES: Fc1cc(SCc2ccccc2)cc(F)c1Br] |
| 2 | 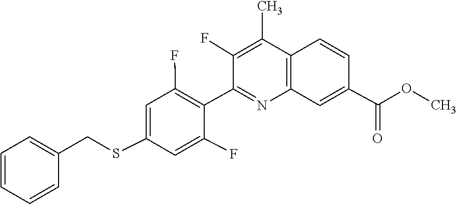 [SMILES: COC(=O)c1ccc2c(C)c(F)c(-c3c(F)cc(SCc4ccccc4)cc3F)nc2c1] |
| 3 | 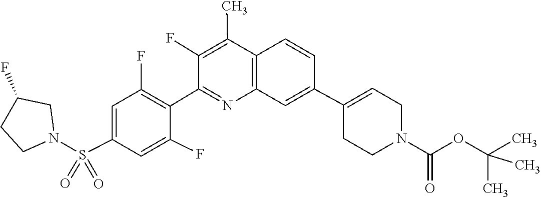 [SMILES: Cc1c(F)c(-c2c(F)cc(S(=O)(=O)N3CC[C@H](F)C3)cc2F)nc2cc(C3=CCN(C(=O)OC(C)(C)C)CC3)ccc12] |
| 4 | 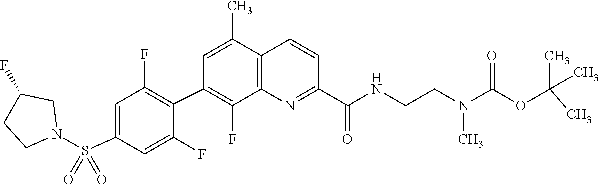 [SMILES: Cc1cc(-c2c(F)cc(S(=O)(=O)N3CC[C@H](F)C3)cc2F)c(F)c2nc(C(=O)NCCN(C)C(=O)OC(C)(C)C)ccc12] |
| 5 | 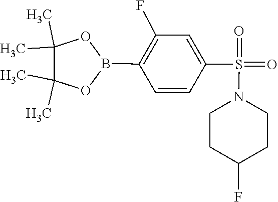 [SMILES: CC1(C)OB(c2ccc(S(=O)(=O)N3CCC(F)CC3)cc2F)OC1(C)C] |

TABLE 4

| PEx | Str |
|---|---|
| 6 | 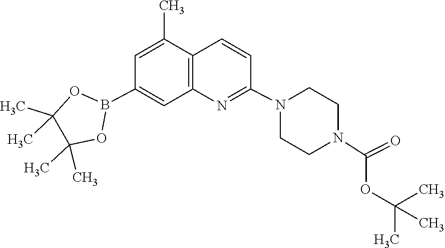 [SMILES: Cc1cc(B2OC(C)(C)C(C)(C)O2)cc2nc(N3CCN(C(=O)OC(C)(C)C)CC3)ccc12] |
| 7 | 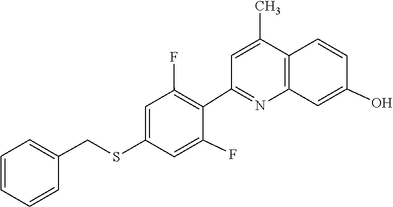 [SMILES: Cc1cc(-c2c(F)cc(SCc3ccccc3)cc2F)nc2cc(O)ccc12] |
| 8 | 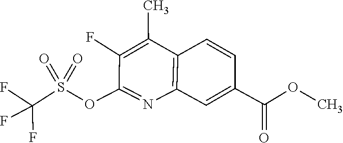 [SMILES: COC(=O)c1ccc2c(C)c(F)c(OS(=O)(=O)C(F)(F)F)nc2c1] |
| 9 | 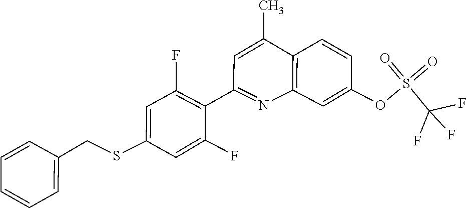 [SMILES: Cc1cc(-c2c(F)cc(SCc3ccccc3)cc2F)nc2cc(OS(=O)(=O)C(F)(F)F)ccc12] |
| 10 | 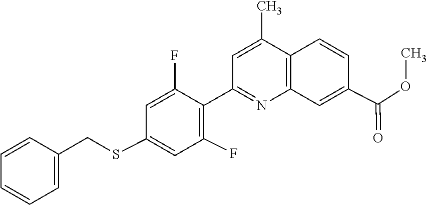 [SMILES: COC(=O)c1ccc2c(C)cc(-c3c(F)cc(SCc4ccccc4)cc3F)nc2c1] |

TABLE 5

| PEx | Str |
|---|---|
| 11 | 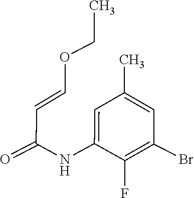 [SMILES: CCO/C=C/C(=O)Nc1cc(C)cc(Br)c1F] |
| 12 | 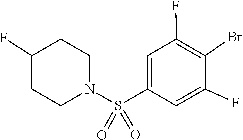 [SMILES: O=S(=O)(c1cc(F)c(Br)c(F)c1)N1CCC(F)CC1] |
| 13 | 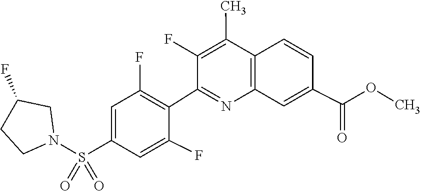 [SMILES: COC(=O)c1ccc2c(C)c(F)c(-c3c(F)cc(S(=O)(=O)N4CC[C@H](F)C4)cc3F)nc2c1] |
| 14 | 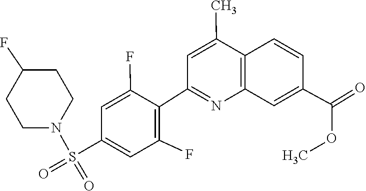 [SMILES: COC(=O)c1ccc2c(C)cc(-c3c(F)cc(S(=O)(=O)N4CCC(F)CC4)cc3F)nc2c1] |
| 15 | 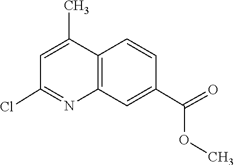 [SMILES: COC(=O)c1ccc2c(C)cc(Cl)nc2c1] |

TABLE 6

| PEx | Str |
|---|---|
| 16 | 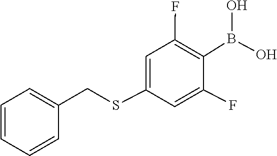 [SMILES: OB(O)c1c(F)cc(SCc2ccccc2)cc1F] |
| 17 | 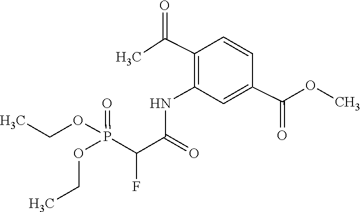 [SMILES: CCOP(=O)(OCC)C(F)C(=O)Nc1cc(C(=O)OC)ccc1C(C)=O] |
| 18 | 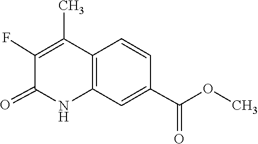 [SMILES: COC(=O)c1ccc2c(C)c(F)c(=O)[nH]c2c1] |
| 19 | 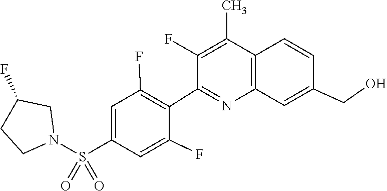 [SMILES: Cc1c(F)c(-c2c(F)cc(S(=O)(=O)N3CC[C@H](F)C3)cc2F)nc2cc(CO)ccc12] |
| 20 | 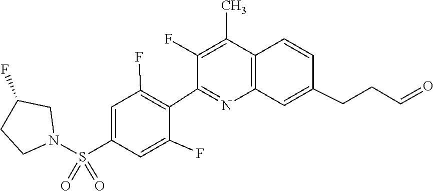 [SMILES: Cc1c(F)c(-c2c(F)cc(S(=O)(=O)N3CC[C@H](F)C3)cc2F)nc2cc(CCC=O)ccc12] |

TABLE 7

| PEx | Str |
|---|---|
| 21 | 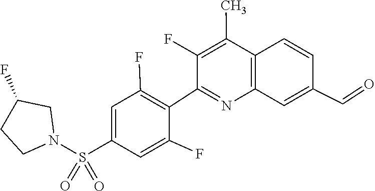 [SMILES: Cc1c(F)c(-c2c(F)cc(S(=O)(=O)N3CC[C@H](F)C3)cc2F)nc2cc(C=O)ccc12] |
| 22 | 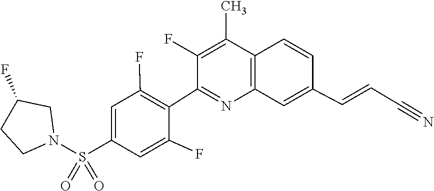 [SMILES: Cc1c(F)c(-c2c(F)cc(S(=O)(=O)N3CC[C@H](F)C3)cc2F)nc2cc(/C=C/C#N)ccc12] |
| 23 | 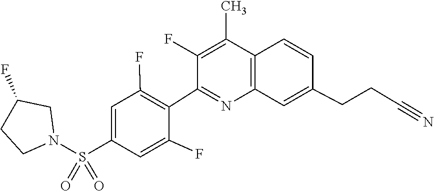 [SMILES: Cc1c(F)c(-c2c(F)cc(S(=O)(=O)N3CC[C@H](F)C3)cc2F)nc2cc(CCC#N)ccc12] |
| 24 | 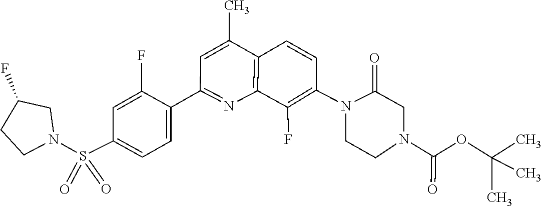 [SMILES: Cc1cc(-c2ccc(S(=O)(=O)N3CC[C@H](F)C3)cc2F)nc2c(F)c(N3CCN(C(=O)OC(C)(C)C)CC3=O)ccc12] |
| 25 | 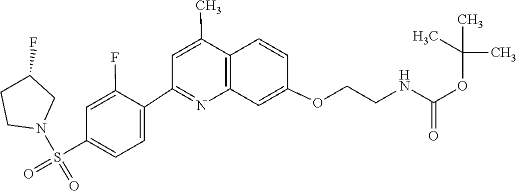 [SMILES: Cc1cc(-c2ccc(S(=O)(=O)N3CC[C@H](F)C3)cc2F)nc2cc(OCCNC(=O)OC(C)(C)C)ccc12] |

TABLE 8

| PEx | Str |
|---|---|
| 26 | 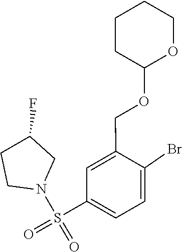 [SMILES: O=S(=O)(c1ccc(Br)c(COC2CCCCO2)c1)N1CC[C@H](F)C1] |
| 27 | 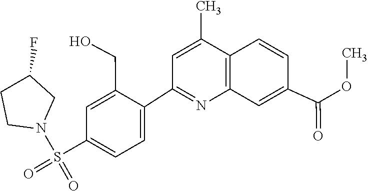 [SMILES: COC(=O)c1ccc2c(C)cc(-c3ccc(S(=O)(=O)N4CC[C@H](F)C4)cc3CO)nc2c1] |
| 28 | 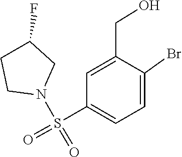 [SMILES: O=S(=O)(c1ccc(Br)c(CO)c1)N1CC[C@H](F)C1] |
| 29 | 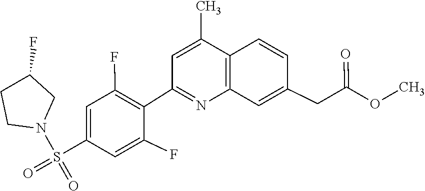 [SMILES: COC(=O)Cc1ccc2c(C)cc(-c3c(F)cc(S(=O)(=O)N4CC[C@H](F)C4)cc3F)nc2c1] |
| 30 | 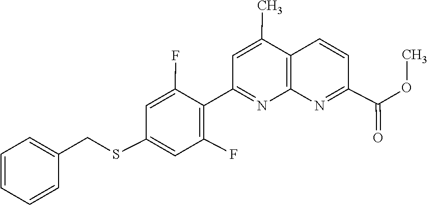 [SMILES: COC(=O)c1ccc2c(C)cc(-c3c(F)cc(SCc4ccccc4)cc3F)nc2n1] |

TABLE 9

| PEx | Str |
|---|---|
| 31 | 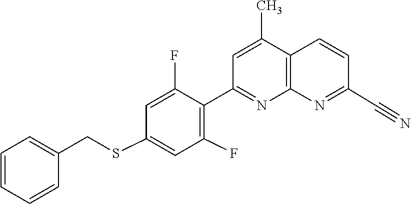 [SMILES: Cc1cc(-c2c(F)cc(SCc3ccccc3)cc2F)nc2nc(C#N)ccc12] |
| 32 | 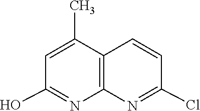 |
| 33 | 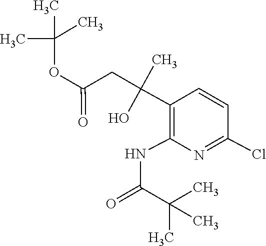 [SMILES: CC(C)(C)OC(=O)CC(C)(O)c1ccc(Cl)nc1NC(=O)C(C)(C)C] |
| 34 | 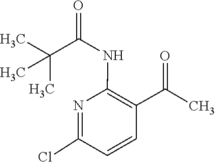 [SMILES: CC(=O)c1ccc(Cl)nc1NC(=O)C(C)(C)C] |
| 35 | 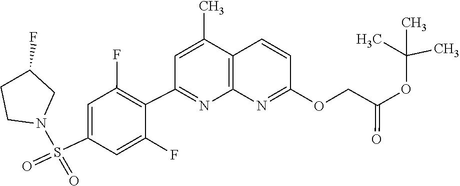 [SMILES: Cc1cc(-c2c(F)cc(S(=O)(=O)N3CC[C@H](F)C3)cc2F)nc2nc(OCC(=O)OC(C)(C)C)ccc12] |

TABLE 10

| PEx | Str |
|---|---|
| 36 | 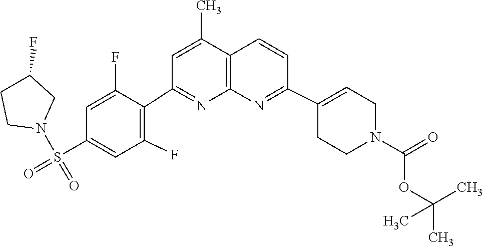 [SMILES: Cc1cc(-c2c(F)cc(S(=O)(=O)N3CC[C@H](F)C3)cc2F)nc2nc(C3=CCN(C(=O)OC(C)(C)C)CC3)ccc12] |
| 37 | 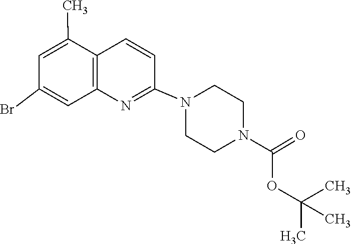 [SMILES: Cc1cc(Br)cc2nc(N3CCN(C(=O)OC(C)(C)C)CC3)ccc12] |
| 38 | 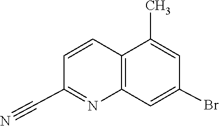 |
| 39 | 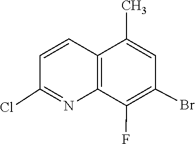 |
| 40 | 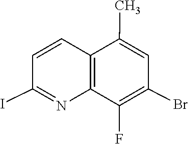 |

TABLE 11

| PEx | Str |
|---|---|
| 41 | 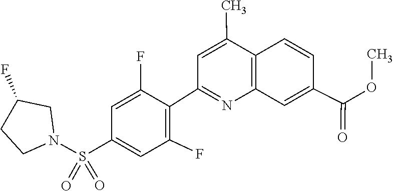 [SMILES: COC(=O)c1ccc2c(C)cc(-c3c(F)cc(S(=O)(=O)N4CC[C@H](F)C4)cc3F)nc2c1] |
| 42 | 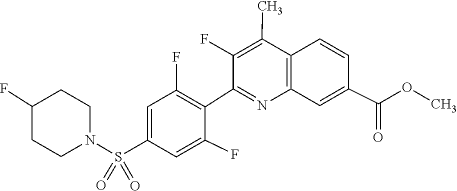 [SMILES: COC(=O)c1ccc2c(C)c(F)c(-c3c(F)cc(S(=O)(=O)N4CCC(F)CC4)cc3F)nc2c1] |
| 44 | 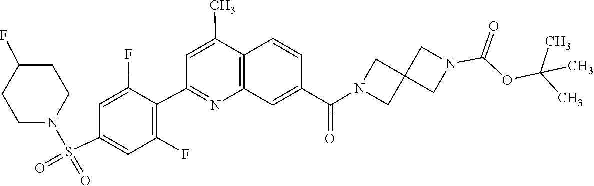 [SMILES: Cc1cc(-c2c(F)cc(S(=O)(=O)N3CCC(F)CC3)cc2F)nc2cc(C(=O)N3CC4(CN(C(=O)OC(C)(C)C)C4)C3)ccc12] |
| 45 | 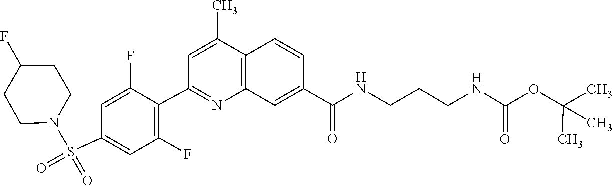 [SMILES: Cc1cc(-c2c(F)cc(S(=O)(=O)N3CCC(F)CC3)cc2F)nc2cc(C(=O)NCCCNC(=O)OC(C)(C)C)ccc12] |
| 46 | 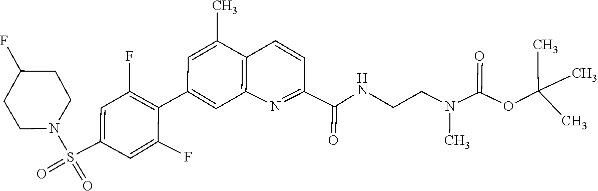 [SMILES: Cc1cc(-c2c(F)cc(S(=O)(=O)N3CCC(F)CC3)cc2F)cc2nc(C(=O)NCCN(C)C(=O)OC(C)(C)C)ccc12] |

TABLE 12

| PEx | Str |
|---|---|
| 47 | 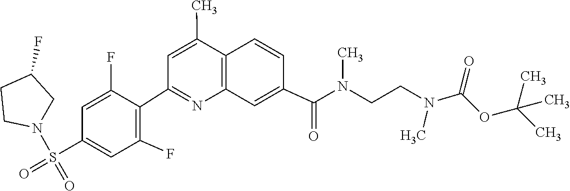 [SMILES: Cc1cc(-c2c(F)cc(S(=O)(=O)N3CC[C@H](F)C3)cc2F)nc2cc(C(=O)N(C)CCN(C)C(=O)OC(C)(C)C)ccc12] |
| 48 | 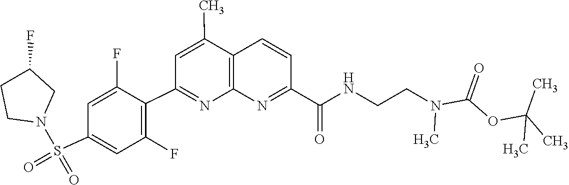 [SMILES: Cc1cc(-c2c(F)cc(S(=O)(=O)N3CC[C@H](F)C3)cc2F)nc2nc(C(=O)NCCN(C)C(=O)OC(C)(C)C)ccc12] |
| 49 | 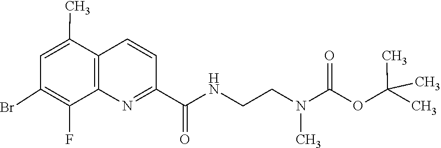 [SMILES: Cc1cc(Br)c(F)c2nc(C(=O)NCCN(C)C(=O)OC(C)(C)C)ccc12] |
| 50 | 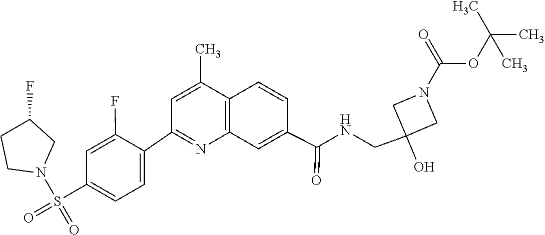 [SMILES: Cc1cc(-c2ccc(S(=O)(=O)N3CC[C@H](F)C3)cc2F)nc2cc(C(=O)NCC3(O)CN(C(=O)OC(C)(C)C)C3)ccc12] |
| 51 | 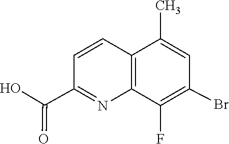 [SMILES: Cc1cc(Br)c(F)c2nc(C(=O)O)ccc12] |

TABLE 13

| PEx | Str |
|---|---|
| 52 | 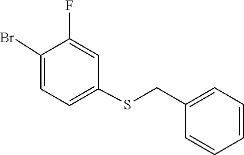 [SMILES: Fc1cc(SCc2ccccc2)ccc1Br] |
| 53 | 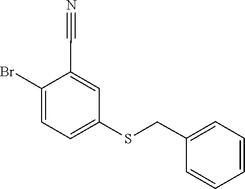 [SMILES: N#Cc1cc(SCc2ccccc2)ccc1Br] |
| 54 | 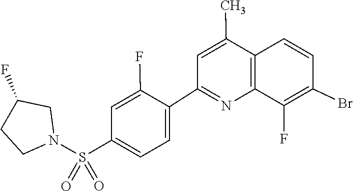 [SMILES: Cc1cc(-c2ccc(S(=O)(=O)N3CC[C@H](F)C3)cc2F)nc2c(F)c(Br)ccc12] |
| 55 | 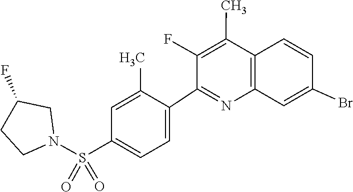 [SMILES: Cc1cc(S(=O)(=O)N2CC[C@H](F)C2)ccc1-c1nc2cc(Br)ccc2c(C)c1F] |
| 56 | 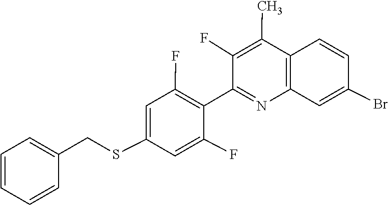 [SMILES: Cc1c(F)c(-c2c(F)cc(SCc3ccccc3)cc2F)nc2cc(Br)ccc12] |

TABLE 14

| PEx | Str |
|---|---|
| 57 | 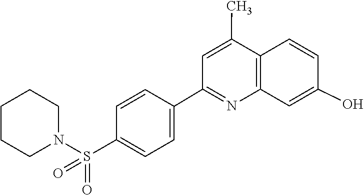 [SMILES: Cc1cc(-c2ccc(S(=O)(=O)N3CCCCC3)cc2)nc2cc(O)ccc12] |
| 58 | 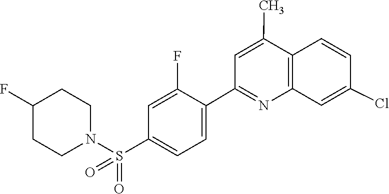 [SMILES: Cc1cc(-c2ccc(S(=O)(=O)N3CCC(F)CC3)cc2F)nc2cc(Cl)ccc12] |
| 59 | 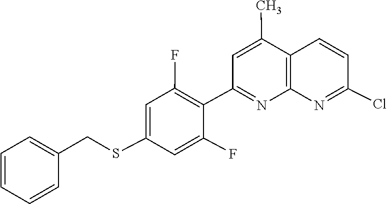 [SMILES: Cc1cc(-c2c(F)cc(SCc3ccccc3)cc2F)nc2nc(Cl)ccc12] |
| 60 | 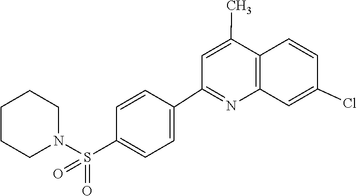 [SMILES: Cc1cc(-c2ccc(S(=O)(=O)N3CCCCC3)cc2)nc2cc(Cl)ccc12] |
| 61 | 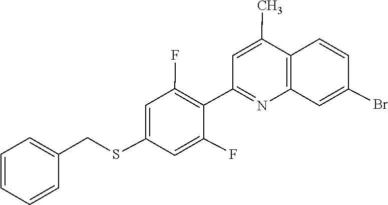 [SMILES: Cc1cc(-c2c(F)cc(SCc3ccccc3)cc2F)nc2cc(Br)ccc12] |

TABLE 15

| PEx | Str |
|---|---|
| 62 | 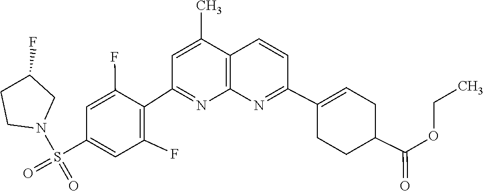 [SMILES: CCOC(=O)C1CC=C(c2ccc3c(C)cc(-c4c(F)cc(S(=O)(=O)N5CC[C@H](F)C5)cc4F)nc3n2)CC1] |
| 63 | 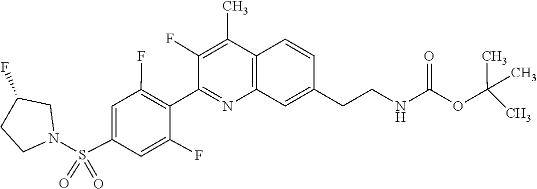 [SMILES: Cc1c(F)c(-c2c(F)cc(S(=O)(=O)N3CC[C@H](F)C3)cc2F)nc2cc(CCNC(=O)OC(C)(C)C)ccc12] |
| 64 | 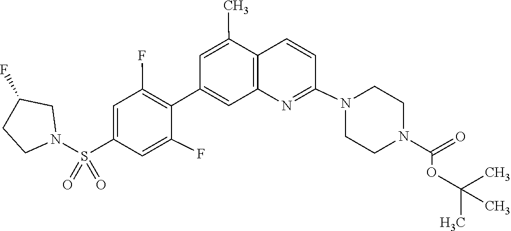 [SMILES: Cc1cc(-c2c(F)cc(S(=O)(=O)N3CC[C@H](F)C3)cc2F)cc2nc(N3CCN(C(=O)OC(C)(C)C)CC3)ccc12] |
| 65 | 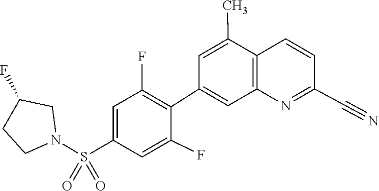 [SMILES: Cc1cc(-c2c(F)cc(S(=O)(=O)N3CC[C@H](F)C3)cc2F)cc2nc(C#N)ccc12] |
| 66 | 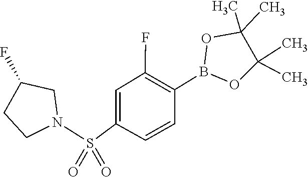 [SMILES: CC1(C)OB(c2ccc(S(=O)(=O)N3CC[C@H](F)C3)cc2F)OC1(C)C] |

TABLE 16

| PEx | Str |
|---|---|
| 67 | 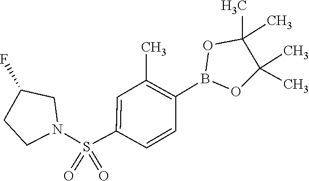 [SMILES: Cc1cc(S(=O)(=O)N2CC[C@H](F)C2)ccc1B1OC(C)(C)C(C)(C)O1] |
| 68 | 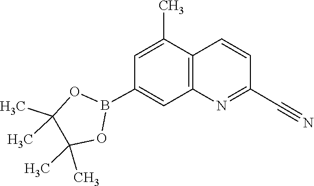 [SMILES: Cc1cc(B2OC(C)(C)C(C)(C)O2)cc2nc(C#N)ccc12] |
| 69 | 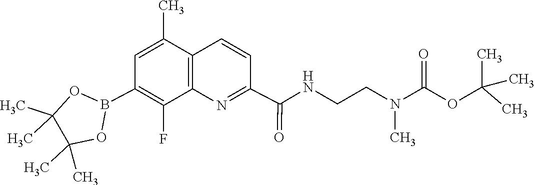 [SMILES: Cc1cc(B2OC(C)(C)C(C)(C)O2)c(F)c2nc(C(=O)NCCN(C)C(=O)OC(C)(C)C)ccc12] |
| 70 | 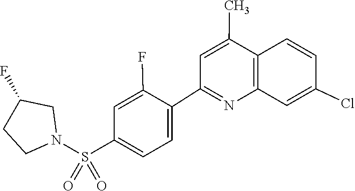 [SMILES: Cc1cc(-c2ccc(S(=O)(=O)N3CC[C@H](F)C3)cc2F)nc2cc(Cl)ccc12] |
| 71 | 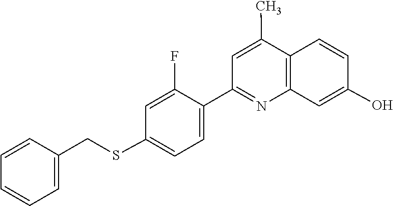 [SMILES: Cc1cc(-c2ccc(SCc3ccccc3)cc2F)nc2cc(O)ccc12] |

TABLE 17

| PEx | Str |
|---|---|
| 72 | 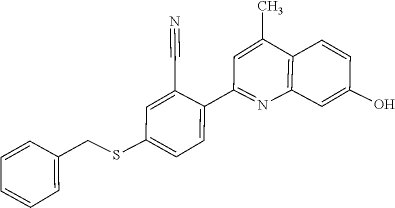 [SMILES: Cc1cc(-c2ccc(SCc3ccccc3)cc2C#N)nc2cc(O)ccc12] |
| 73 | 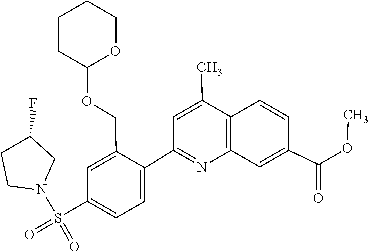 [SMILES: COC(=O)c1ccc2c(C)cc(-c3ccc(S(=O)(=O)N4CC[C@H](F)C4)cc3COC3CCCCO3)nc2c1] |
| 74 | 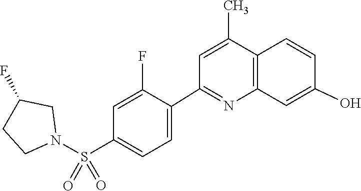 [SMILES: Cc1cc(-c2ccc(S(=O)(=O)N3CC[C@H](F)C3)cc2F)nc2cc(O)ccc12] |
| 75 | 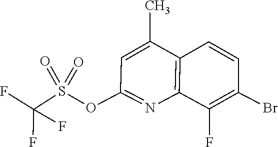 [SMILES: Cc1cc(OS(=O)(=O)C(F)(F)F)nc2c(F)c(Br)ccc12] |
| 76 | 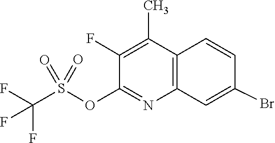 [SMILES: Cc1c(F)c(OS(=O)(=O)C(F)(F)F)nc2cc(Br)ccc12] |

TABLE 18

| PEx | Str |
|---|---|
| 77 | 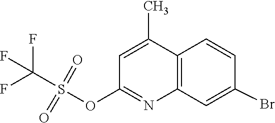 [SMILES: Cc1cc(OS(=O)(=O)C(F)(F)F)nc2cc(Br)ccc12] |
| 78 | 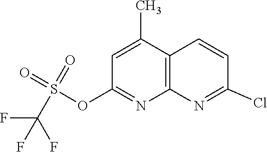 [SMILES: Cc1cc(OS(=O)(=O)C(F)(F)F)nc2nc(Cl)ccc12] |
| 79 | 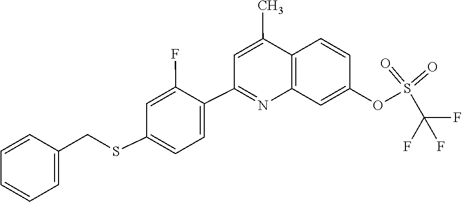 [SMILES: Cc1cc(-c2ccc(SCc3ccccc3)cc2F)nc2cc(OS(=O)(=O)C(F)(F)F)ccc12] |
| 80 | 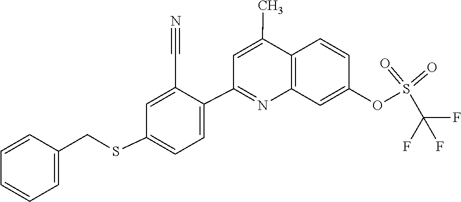 [SMILES: Cc1cc(-c2ccc(SCc3ccccc3)cc2C#N)nc2cc(OS(=O)(=O)C(F)(F)F)ccc12] |
| 81 | 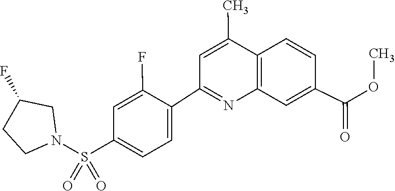 [SMILES: COC(=O)c1ccc2c(C)cc(-c3ccc(S(=O)(=O)N4CC[C@H](F)C4)cc3F)nc2c1] |

TABLE 19

| PEx | Str |
|---|---|
| 82 | 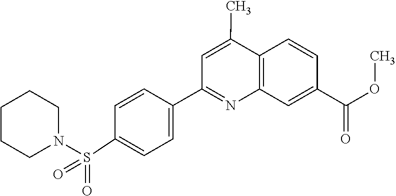 [SMILES: COC(=O)c1ccc2c(C)cc(-c3ccc(S(=O)(=O)N4CCCCC4)cc3)nc2c1] |
| 83 | 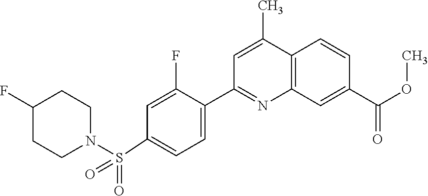 [SMILES: COC(=O)c1ccc2c(C)cc(-c3ccc(S(=O)(=O)N4CCC(F)CC4)cc3F)nc2c1] |
| 84 | 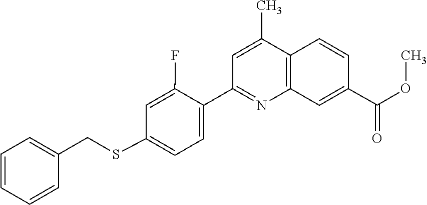 [SMILES: COC(=O)c1ccc2c(C)cc(-c3ccc(SCc4ccccc4)cc3F)nc2c1] |
| 85 | 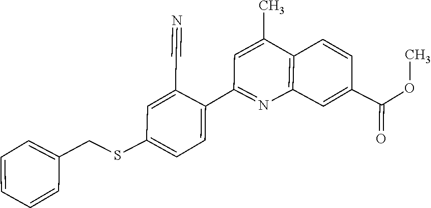 [SMILES: COC(=O)c1ccc2c(C)cc(-c3ccc(SCc4ccccc4)cc3C#N)nc2c1] |
| 86 | 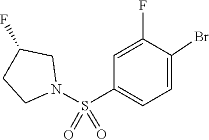 [SMILES: O=S(=O)(c1ccc(Br)c(F)c1)N1CC[C@H](F)C1] |

TABLE 20

| PEx | Str |
|---|---|
| 87 | 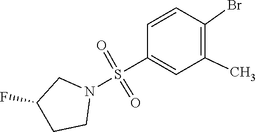 [SMILES: Cc1cc(S(=O)(=O)N2CC[C@H](F)C2)ccc1Br] |
| 88 | 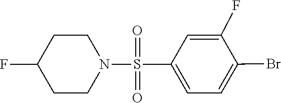 [SMILES: O=S(=O)(c1ccc(Br)c(F)c1)N1CCC(F)CC1] |
| 89 | 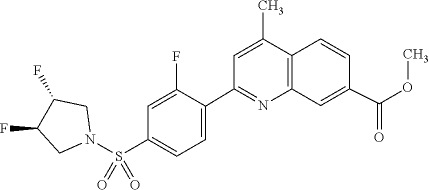 [SMILES: COC(=O)c1ccc2c(C)cc(-c3ccc(S(=O)(=O)N4C[C@@H](F)[C@H](F)C4)cc3F)nc2c1] |
| 90 | 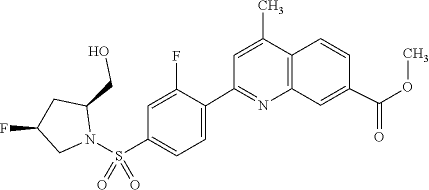 [SMILES: COC(=O)c1ccc2c(C)cc(-c3ccc(S(=O)(=O)N4C[C@@H](F)C[C@H]4CO)cc3F)nc2c1] |
| 91 | 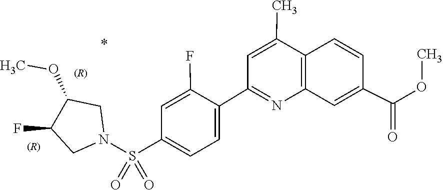 |

TABLE 21

| PEx | Str |
|---|---|
| 92 | 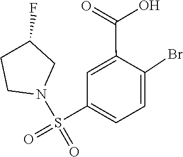 [SMILES: O=C(O)c1cc(S(=O)(=O)N2CC[C@H](F)C2)ccc1Br] |
| 93 | 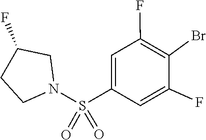 [SMILES: O=S(=O)(c1cc(F)c(Br)c(F)c1)N1CC[C@H](F)C1] |
| 94 | 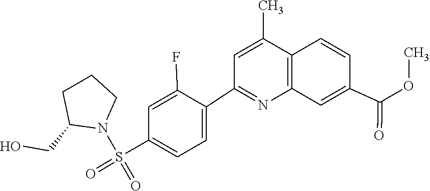 [SMILES: COC(=O)c1ccc2c(C)cc(-c3ccc(S(=O)(=O)N4CCC[C@H]4CO)cc3F)nc2c1] |
| 95 | 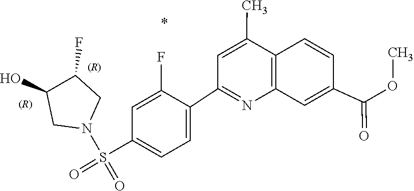 |
| 96 | 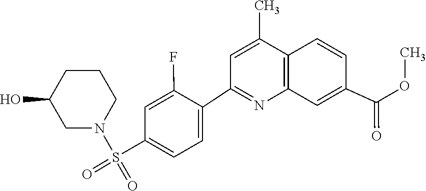 [SMILES: COC(=O)c1ccc2c(C)cc(-c3ccc(S(=O)(=O)N4CCC[C@H](O)C4)cc3F)nc2c1] |

TABLE 22

| PEx | Str |
|---|---|
| 97 | 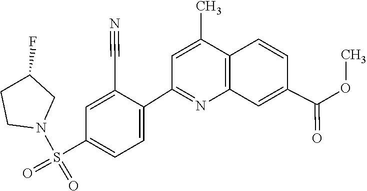 [SMILES: COC(=O)c1ccc2c(C)cc(-c3ccc(S(=O)(=O)N4CC[C@H](F)C4)cc3C#N)nc2c1] |
| 98 | 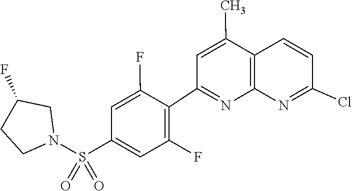 [SMILES: Cc1cc(-c2c(F)cc(S(=O)(=O)N3CC[C@H](F)C3)cc2F)nc2nc(Cl)ccc12] |
| 99 | 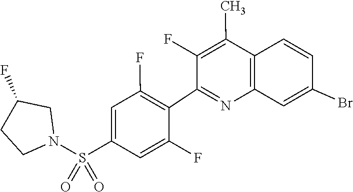 [SMILES: Cc1c(F)c(-c2c(F)cc(S(=O)(=O)N3CC[C@H](F)C3)cc2F)nc2cc(Br)ccc12] |
| 100 | 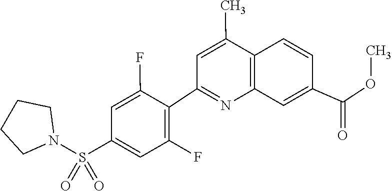 [SMILES: COC(=O)c1ccc2c(C)cc(-c3c(F)cc(S(=O)(=O)N4CCCC4)cc3F)nc2c1] |
| 101 | 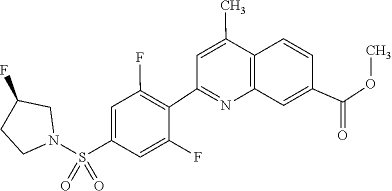 [SMILES: COC(=O)c1ccc2c(C)cc(-c3c(F)cc(S(=O)(=O)N4CC[C@@H](F)C4)cc3F)nc2c1] |

TABLE 23

| PEx | Str |
|---|---|
| 102 | 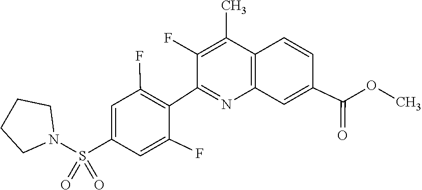 [SMILES: COC(=O)c1ccc2c(C)c(F)c(-c3c(F)cc(S(=O)(=O)N4CCCC4)cc3F)nc2c1] |
| 103 | 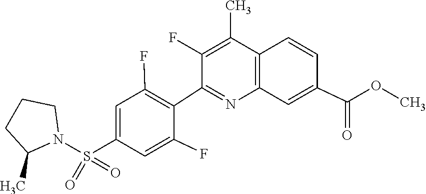 [SMILES: COC(=O)c1ccc2c(C)c(F)c(-c3c(F)cc(S(=O)(=O)N4CCC[C@@H]4C)cc3F)nc2c1] |
| 104 | 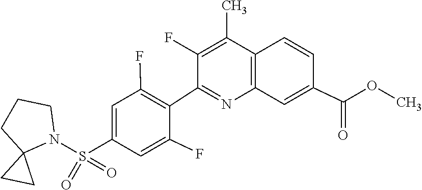 [SMILES: COC(=O)c1ccc2c(C)c(F)c(-c3c(F)cc(S(=O)(=O)N4CCCC45CC5)cc3F)nc2c1] |
| 105 | 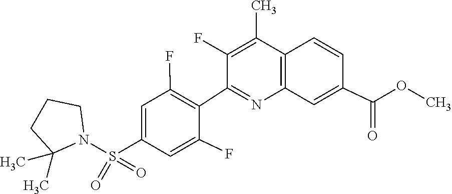 |
| 106 | 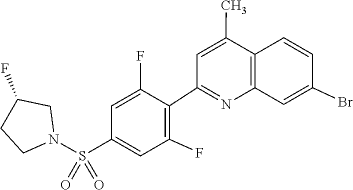 [SMILES: Cc1cc(-c2c(F)cc(S(=O)(=O)N3CC[C@H](F)C3)cc2F)nc2cc(Br)ccc12] |

TABLE 24

| PEx | Str |
|---|---|
| 107 | 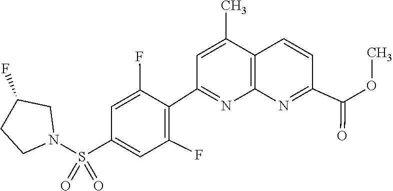 [SMILES: COC(=O)c1ccc2c(C)cc(-c3c(F)cc(S(=O)(=O)N4CC[C@H](F)C4)cc3F)nc2n1] |
| 108 | 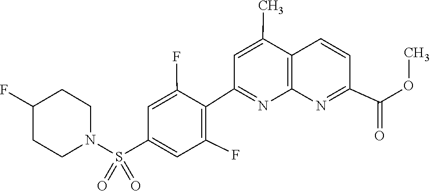 [SMILES: COC(=O)c1ccc2c(C)cc(-c3c(F)cc(S(=O)(=O)N4CCC(F)CC4)cc3F)nc2n1] |
| 109 | 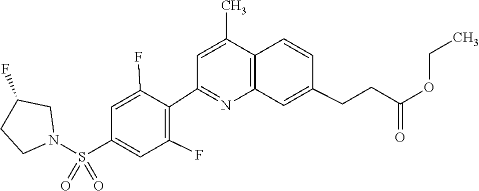 [SMILES: CCOC(=O)CCc1ccc2c(C)cc(-c3c(F)cc(S(=O)(=O)N4CC[C@H](F)C4)cc3F)nc2c1] |
| 110 | 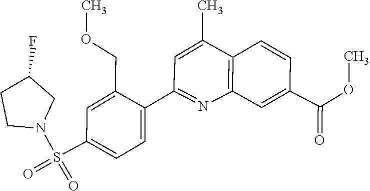 [SMILES: COCc1cc(S(=O)(=O)N2CC[C@H](F)C2)ccc1-c1cc(C)c2ccc(C(=O)OC)cc2n1] |
| 111 | 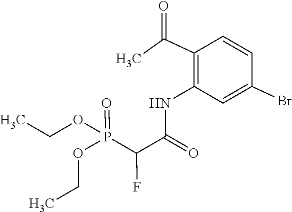 [SMILES: CCOP(=O)(OCC)C(F)C(=O)Nc1cc(Br)ccc1C(C)=O] |

TABLE 25

| PEx | Str |
|---|---|
| 112 | 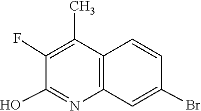 |
| 113 | 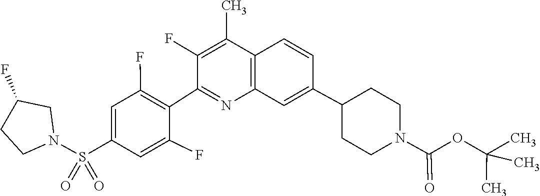 [SMILES: Cc1c(F)c(-c2c(F)cc(S(=O)(=O)N3CC[C@H](F)C3)cc2F)nc2cc(C3CCN(C(=O)OC(C)(C)C)CC3)ccc12] |
| 114 | 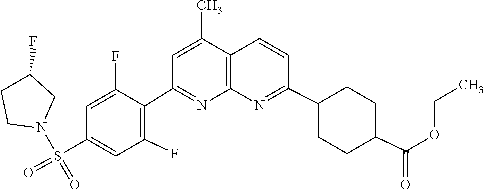 [SMILES: CCOC(=O)C1CCC(c2ccc3c(C)cc(-c4c(F)cc(S(=O)(=O)N5CC[C@H](F)C5)cc4F)nc3n2)CC1] |
| 115 | 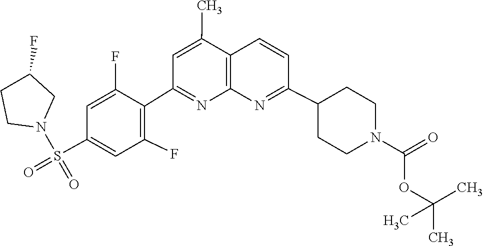 [SMILES: Cc1cc(-c2c(F)cc(S(=O)(=O)N3CC[C@H](F)C3)cc2F)nc2nc(C3CCN(C(=O)OC(C)(C)C)CC3)ccc12] |
| 116 | 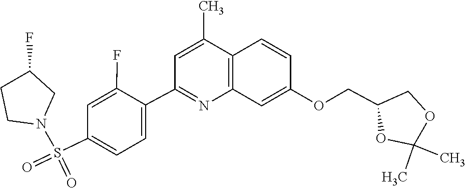 [SMILES: Cc1cc(-c2ccc(S(=O)(=O)N3CC[C@H](F)C3)cc2F)nc2cc(OC[C@@H]3COC(C)(C)O3)ccc12] |

TABLE 26

| PEx | Str |
|---|---|
| 117 | 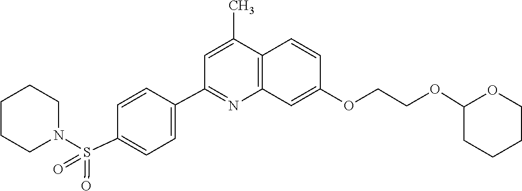 [SMILES: Cc1cc(-c2ccc(S(=O)(=O)N3CCCCC3)cc2)nc2cc(OCCOC3CCCCO3)ccc12] |
| 118 | 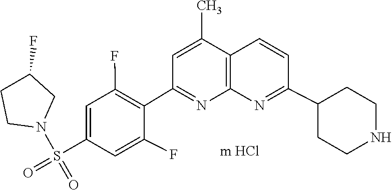 [SMILES: Cc1cc(-c2c(F)cc(S(=O)(=O)N3CC[C@H](F)C3)cc2F)nc2nc(C3CCNCC3)ccc12] |
| 119 | 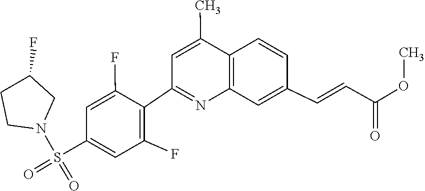 [SMILES: COC(=O)/C=C/c1ccc2c(C)cc(-c3c(F)cc(S(=O)(=O)N4CC[C@H](F)C4)cc3F)nc2c1] |
| 120 | 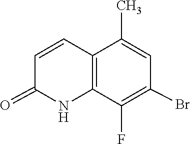 |
| 121 | 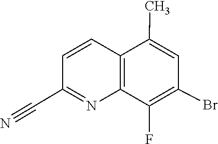 |

TABLE 27

| PEx | Str |
|---|---|
| 122 | 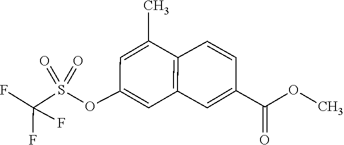 [SMILES: COC(=O)c1ccc2c(C)cc(OS(=O)(=O)C(F)(F)F)cc2c1] |
| 123 | 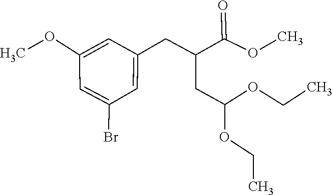 [SMILES: CCOC(CC(Cc1cc(Br)cc(OC)c1)C(=O)OC)OCC] |
| 124 | 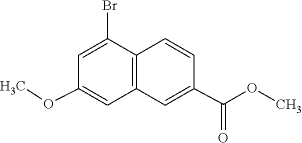 |
| 125 | 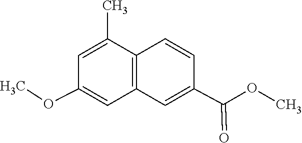 |

TABLE 28

| PEx | Str |
|---|---|
| 126 | 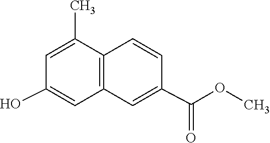 [SMILES: COC(=O)c1ccc2c(C)cc(O)cc2c1] |
| 127 | 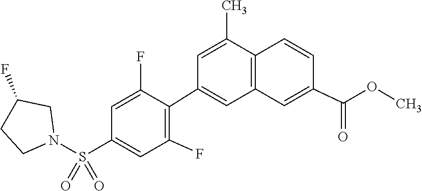 [SMILES: COC(=O)c1ccc2c(C)cc(-c3c(F)cc(S(=O)(=O)N4CC[C@H](F)C4)cc3F)cc2c1] |

TABLE 29

| PEx | PSyn | DAT |
|---|---|---|
| 1 | P1 | ESI−; 313.1 |
| 2 | P2 | ESI+; 454.1 |
| 3 | P3 | ESI+; 606.2 |
| 4 | P4 | ESI+; 625.3 |
| 5 | P5 | 1H NMR (500 MHz, DMSO-d6) δ ppm 1.32 (12H, s), 1.73-1.99 (4H, |
| | | m), 2.93-2.99 (2H, m), 3.05-3.12 (2H, m), 4.66-4.83 (1H, m), 7.52 (1H, |
| | | dd, J = 8.4, 1.4 Hz), 7.60 (1H, dd, J = 7.7, 1.5 Hz), 7.90 (1H, dd, J = 8.0, |
| | | 5.8 Hz). |
| 6 | P6 | ESI+; 454.3 |
| 7 | P7 | ESI+; 394.2 |
| 8 | P8 | ESI+; 368.1 |
| 9 | P9 | ESI+; 526.2 |
| 10 | P10 | ESI+; 436.2 |
| 11 | P11 | ESI+; 304.0 |
| 12 | P12 | ESI+; 358.0 |
| 13 | P13 | ESI+; 483.1 |
| 14 | P14 | ESI+; 479.2 |
| 15 | P15 | ESI+; 236.1 |
| 16 | P16 | ESI+; 303.0 [M + Na]+ |
| 17 | P17 | ESI+; 390.1 |
| 18 | P18 | ESI+; 236.1 |
| 19 | P19 | ESI+; 455.1 |
| 20 | P20 | ESI+; 481.1 |
| 21 | P21 | ESI+; 453.1 |
| 22 | P22 | ESI+; 476.1 |

TABLE 30

| PEx | PSyn | DAT |
|---|---|---|
| 23 | P23 | ESI+; 478.2 |
| 24 | P24 | 1H NMR (400 MHz, CDCl₃) δ ppm 1.53 (9H, s), 1.89-2.08 (1H, m), |
| | | 2.15-2.30 (1H, m), 2.80 (3H, s), 3.33-3.45 (2H, m), 3.50-3.68 (4H, m), |
| | | 3.80-3.95 (2H, m), 4.36 (2H, s), 5.11-5.28 (1H, m), 7.54 (1H, dd, J = |
| | | 8.8, 6.8 Hz), 7.70 (1H, dd, J = 10.4, 1.6 Hz), 7.79 (1H, dd, J = 8.0, 1.6 |
| | | Hz), 7.84-7.90 (2H, m), 8.35 (1H, t, J = 7.6 Hz). |
| 25 | P25 | ESI+; 548.3 |
| 26 | P26 | ESI+; 444.1, 446.1 [M + Na]+ |
| 27 | P27 | ESI+; 459.2 |
| 28 | P28 | ESI+; 338.0 |
| 29 | P29 | ESI+; 479.2 |
| 30 | P30 | ESI+; 437.2 |
| 31 | P31 | ESI+; 404.1 |
| 32 | P32 | ESI+; 195.0 |
| 33 | P33 | ESI+; 371.2, 373.2 |
| 34 | P34 | ESI+; 255.1 |
| 35 | P35 | ESI+; 538.2 |
| 36 | P36 | ESI+; 589.3 |
| 37 | P37 | ESI+; 408.2 |
| 38 | P38 | ESI+; 249.0 |
| 39 | P39 | ESI+; 273.9, 275.8 |
| 40 | P40 | ESI+; 365.9 |
| 41 | P41 | ESI+; 465.3 |
| 42 | P42 | ESI+; 497.1 |
| 44 | E3 | ESI+; 645.6 |
| 45 | E3 | ESI+; 621.3 |

TABLE 31

| PEx | PSyn | DAT |
|---|---|---|
| 46 | E3 | ESI+; 622.3 |
| 47 | E3 | ESI+; 621.2 |
| 48 | E3 | ESI+; 608.4 |
| 49 | E3 | ESI+; 440.2 |
| 50 | E3 | ESI+; 617.4 |
| 51 | E8 | ESI+; 284.0 |
| 52 | P1 | ESI−; 295.0 |
| 53 | P1 | ESI−; 301.9, 303.9 |
| 54 | P2 | 1H NMR (400 MHz, CDCl3) δ ppm 1.93-2.10 (1H, m), 2.16-2.30 (1H, |
| | | m), 2.80 (3H, s), 3.34-3.45 (1H, m), 3.50-3.70 (3H, m), 5.10-5.30 (1H, |
| | | m), 7.68-7.74 (3H, m), 7.79 (1H, dd, J = 8.0, 1.6 Hz), 7.86 (1H, d, J = |
| | | 1.6 Hz), 8.36 (1H, t, J = 8.0 Hz). |
| 55 | P2 | ESI+; 482.9 |
| 56 | P2 | ESI+; 474.1, 476.1 |
| 57 | P2 | ESI+; 383.3 |
| 58 | P2 | ESI+; 437.1, 439.1 |
| 59 | P2 | ESI+; 413.1, 415.1 |
| 60 | P2 | ESI+; 401.2, 403.2 |
| 61 | P2 | ESI+; 458.1 |
| 62 | P3 | ESI+; 560.3 |
| 63 | P3 | ESI+; 568.3 |
| 64 | P4 | ESI+; 591.2 |
| 65 | P4 | ESI+; 432.2 |

TABLE 32

| PEx | PSyn | DAT |
|---|---|---|
| 66 | P5 | 1H NMR (400 MHz, CDCl3) δ ppm 1.38 (12H, s), 1.89-2.04 (1H, m), |
| | | 2.07-2.25 (1H, m), 3.25-3.32 (1H, m), 3.44-3.57 (3H, m), 5.05-5.28 |
| | | (1H, m), 7.48 (1H, dd, J = 8.0, 1.6 Hz), 7.59 (1H, dd, J = 8.0, 1.6 Hz), |
| | | 7.90 (1H, dd, J = 8.8, 2.0 Hz). |
| 67 | P5 | ESI+; 370.2 |
| 68 | P6 | ESI+; 295.1 |
| 69 | P6 | ESI+; 488.3 |
| 70 | P7 | ESI+; 423.2, 425.2 |
| 71 | P7 | ESI+; 376.2 |
| 72 | P7 | ESI+; 383.2 |
| 73 | P7 | ESI+; 543.3 |
| 74 | P7 | ESI+; 405.1 |
| 75 | P8 | ESI+; 389.8 |
| 76 | P8 | ESI+; 388.0 |
| 77 | P8 | ESI+; 372.0 |
| 78 | P8 | ESI+; 327.0 |
| 79 | P9 | ESI+; 508.1 |
| 80 | P9 | ESI+; 515.2 |
| 81 | P10 | ESI+; 447.3 |
| 82 | P10 | ESI+; 425.2 |
| 83 | P10 | ESI+; 461.3 |
| 84 | P10 | ESI+; 418.2 |
| 85 | P10 | ESI+; 425.2 |
| 86 | P12 | ESI+; 328.0 |
| 87 | P12 | ESI+; 324.0 |
| 88 | P12 | ESI+; 342.0 |

TABLE 33

| PEx | PSyn | DAT |
|---|---|---|
| 89 | P13 | ESI+; 465.2 |
| 90 | P13 | ESI+; 477.2 |
| 91 | P13 | ESI+; 477.2 |
| 92 | P12 | ESI+; 352.1 |
| 93 | P12 | ESI+; 346.0 |
| 94 | P13 | ESI+; 459.2 |
| 95 | P13 | ESI+; 463.2 |
| 96 | P13 | ESI+; 459.2 |
| 97 | P13 | ESI+; 454.3 |
| 98 | P13 | ESI+; 442.1, 444.0 |
| 99 | P13 | ESI+; 503.0, 505.0 |
| 100 | P13 | ESI+; 447.2 |
| 101 | P13 | ESI+; 465.3 |
| 102 | P13 | ESI+; 465.2 |
| 103 | P13 | ESI+; 479.1 |
| 104 | P13 | ESI+; 491.2 |
| 105 | P13 | ESI+; 493.2 |
| 106 | P13 | ESI+; 487.0 |
| 107 | P13 | ESI+; 466.1 |
| 108 | P13 | ESI+; 480.2 |
| 109 | P14 | ESI+; 507.2 |
| 110 | P110 | ESI+; 473.2 |
| 111 | P17 | ESI+; 412.0 |
| 112 | P18 | ESI+; 258.0 |
| 113 | P23 | ESI+; 608.3 |
| 114 | P23 | ESI+; 562.3 |

TABLE 34

| PEx | PSyn | DAT |
|---|---|---|
| 115 | P23 | ESI+; 591.3 |
| 116 | P25 | ESI+; 519.3 |
| 117 | P25 | ESI+; 511.4 |
| 118 | E13 | ESI+; 491.2 |
| 119 | P29 | ESI+; 491.2 |
| 120 | P120 | ESI+; 256.0 |
| 121 | P38 | ESI+; 265.0 |
| 122 | P8 | ESI+; 349.3 |
| 123 | P123 | ESI+; 413.2 [M + Na]+ |
| 124 | P124 | ESI+; 295.1, 297.1 |
| 125 | P125 | ESI+; 231.2 |
| 126 | P126 | ESI+; 217.2 |
| 127 | P127 | ESI+; 464.4 |

TABLE 35

| Ex | Str |
|---|---|
| 1 | 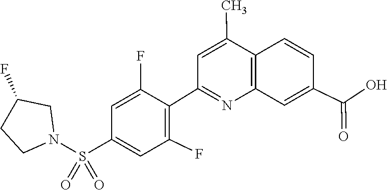 [SMILES: Cc1cc(-c2c(F)cc(S(=O)(=O)N3CC[C@H](F)C3)cc2F)nc2cc(C(=O)O)ccc12] |
| 2 | 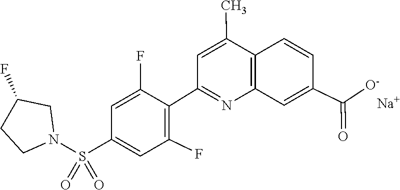 [SMILES: Cc1cc(-c2c(F)cc(S(=O)(=O)N3CC[C@H](F)C3)cc2F)nc2cc(C(=O)[O-])ccc12 ; [Na+]] |
| 3 | 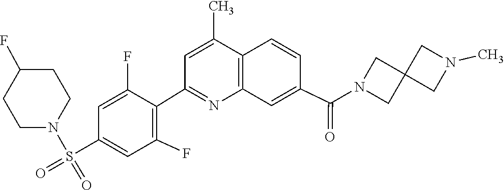 [SMILES: Cc1cc(-c2c(F)cc(S(=O)(=O)N3CCC(F)CC3)cc2F)nc2cc(C(=O)N3CC4(CN(C)C4)C3)ccc12] |
| 4 | 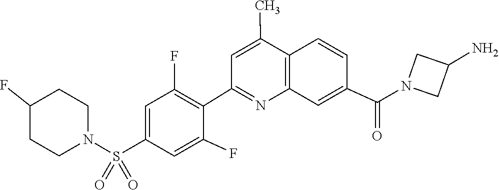 [SMILES: Cc1cc(-c2c(F)cc(S(=O)(=O)N3CCC(F)CC3)cc2F)nc2cc(C(=O)N3CC(N)C3)ccc12] |

TABLE 36

| Ex | Str |
|---|---|
| 5 | 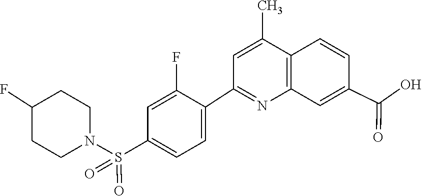 [SMILES: Cc1cc(-c2ccc(S(=O)(=O)N3CCC(F)CC3)cc2F)nc2cc(C(=O)O)ccc12] |
| 6 | 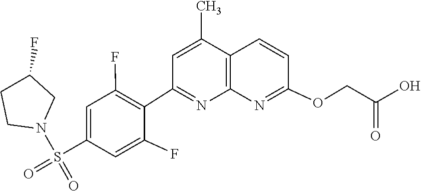 [SMILES: Cc1cc(-c2c(F)cc(S(=O)(=O)N3CC[C@H](F)C3)cc2F)nc2nc(OCC(=O)O)ccc12] |
| 7 | 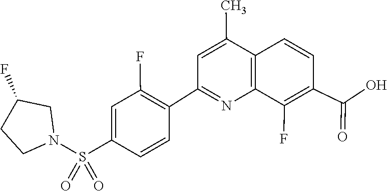 [SMILES: Cc1cc(-c2ccc(S(=O)(=O)N3CC[C@H](F)C3)cc2F)nc2c(F)c(C(=O)O)ccc12] |
| 8 | 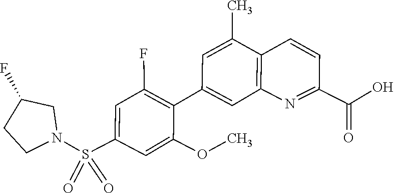 [SMILES: COc1cc(S(=O)(=O)N2CC[C@H](F)C2)cc(F)c1-c1cc(C)c2ccc(C(=O)O)nc2c1] |

TABLE 37

| Ex | Str |
|---|---|
| 9 | 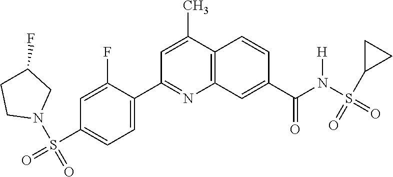 [SMILES: Cc1cc(-c2ccc(S(=O)(=O)N3CC[C@H](F)C3)cc2F)nc2cc(C(=O)NS(=O)(=O)C3CC3)ccc12] |
| 10 | 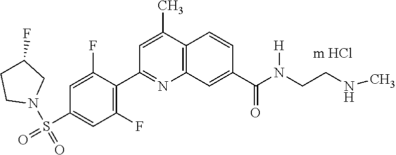 [SMILES: CNCCNC(=O)c1ccc2c(C)cc(-c3c(F)cc(S(=O)(=O)N4CC[C@H](F)C4)cc3F)nc2c1] |
| 11 | 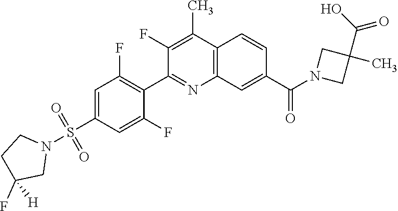 [SMILES: Cc1c(F)c(-c2c(F)cc(S(=O)(=O)N3CC[C@H](F)C3)cc2F)nc2cc(C(=O)N3CC(C)(C(=O)O)C3)ccc12] |
| 12 | 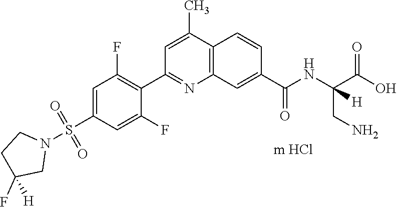 [SMILES: Cc1cc(-c2c(F)cc(S(=O)(=O)N3CC[C@H](F)C3)cc2F)nc2cc(C(=O)N[C@@H](CN)C(=O)O)ccc12] |

TABLE 38

| Ex | Str |
|---|---|
| 13 | 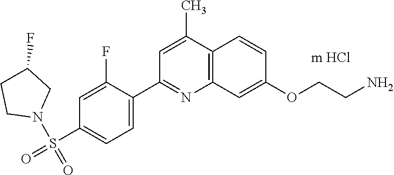 [SMILES: Cc1cc(-c2ccc(S(=O)(=O)N3CC[C@H](F)C3)cc2F)nc2cc(OCCN)ccc12] |
| 14 | 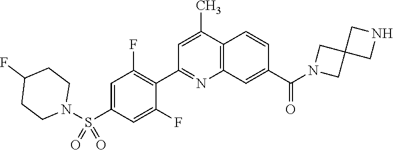 [SMILES: Cc1cc(-c2c(F)cc(S(=O)(=O)N3CCC(F)CC3)cc2F)nc2cc(C(=O)N3CC4(CNC4)C3)ccc12] |
| 15 | 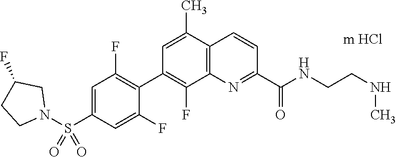 [SMILES: CNCCNC(=O)c1ccc2c(C)cc(-c3c(F)cc(S(=O)(=O)N4CC[C@H](F)C4)cc3F)c(F)c2n1] |
| 16 | 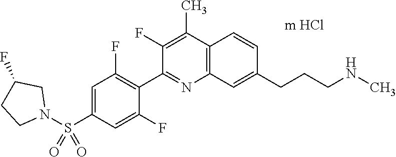 [SMILES: CNCCCc1ccc2c(C)c(F)c(-c3c(F)cc(S(=O)(=O)N4CC[C@H](F)C4)cc3F)nc2c1] |

TABLE 39

| Ex | Str |
|---|---|
| 17 | 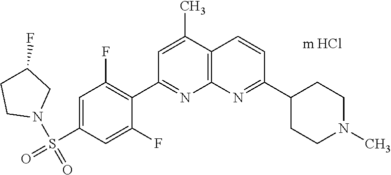 [SMILES: Cc1cc(-c2c(F)cc(S(=O)(=O)N3CC[C@H](F)C3)cc2F)nc2nc(C3CCN(C)CC3)ccc12] |
| 18 | 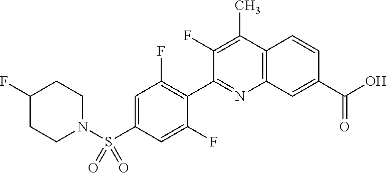 [SMILES: Cc1c(F)c(-c2c(F)cc(S(=O)(=O)N3CCC(F)CC3)cc2F)nc2cc(C(=O)O)ccc12] |
| 19 | 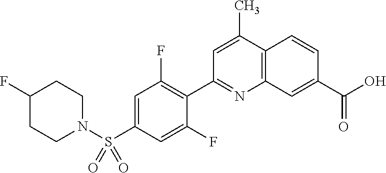 [SMILES: Cc1cc(-c2c(F)cc(S(=O)(=O)N3CCC(F)CC3)cc2F)nc2cc(C(=O)O)ccc12] |
| 20 | 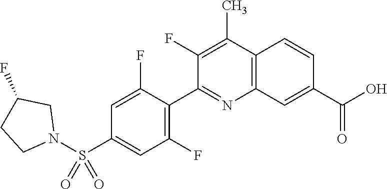 [SMILES: Cc1c(F)c(-c2c(F)cc(S(=O)(=O)N3CC[C@H](F)C3)cc2F)nc2cc(C(=O)O)ccc12] |

TABLE 40

| Ex | Str |
|---|---|
| 21 | 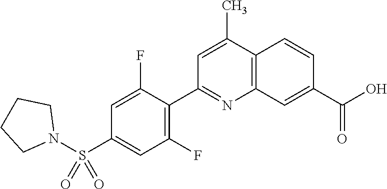 [SMILES: Cc1cc(-c2c(F)cc(S(=O)(=O)N3CCCC3)cc2F)nc2cc(C(=O)O)ccc12] |
| 22 | 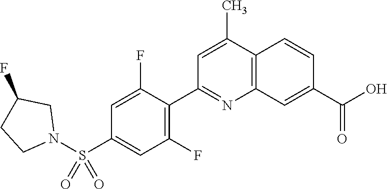 [SMILES: Cc1cc(-c2c(F)cc(S(=O)(=O)N3CC[C@@H](F)C3)cc2F)nc2cc(C(=O)O)ccc12] |
| 23 | 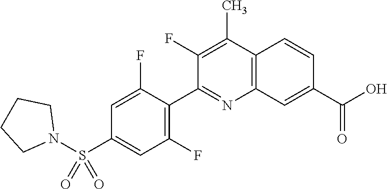 [SMILES: Cc1c(F)c(-c2c(F)cc(S(=O)(=O)N3CCCC3)cc2F)nc2cc(C(=O)O)ccc12] |
| 24 | 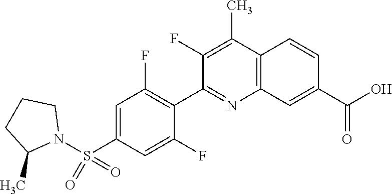 [SMILES: Cc1c(F)c(-c2c(F)cc(S(=O)(=O)N3CCC[C@@H]3C)cc2F)nc2cc(C(=O)O)ccc12] |

TABLE 41

| Ex | Str |
|---|---|
| 25 | 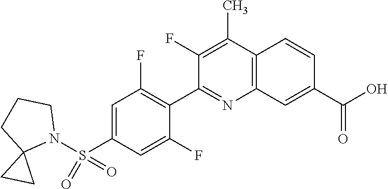 [SMILES: Cc1c(F)c(-c2c(F)cc(S(=O)(=O)N3CCCC34CC4)cc2F)nc2cc(C(=O)O)ccc12] |
| 26 | 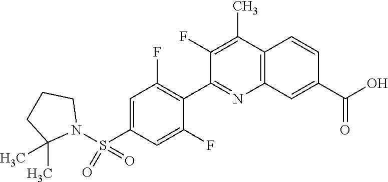 [SMILES: Cc1c(F)c(-c2c(F)cc(S(=O)(=O)N3CCCC3(C)C)cc2F)nc2cc(C(=O)O)ccc12] |
| 27 | 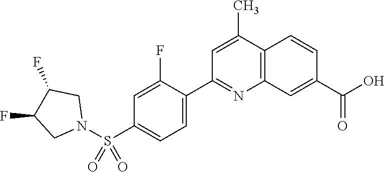 [SMILES: Cc1cc(-c2ccc(S(=O)(=O)N3C[C@@H](F)[C@H](F)C3)cc2F)nc2cc(C(=O)O)ccc12] |
| 28 | 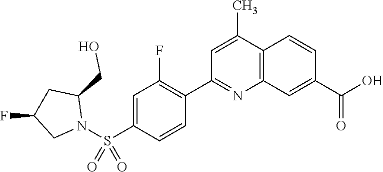 [SMILES: Cc1cc(-c2ccc(S(=O)(=O)N3C[C@@H](F)C[C@H]3CO)cc2F)nc2cc(C(=O)O)ccc12] |

TABLE 42

| Ex | Str |
|---|---|
| 29 | 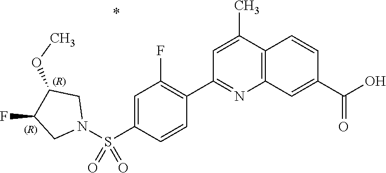 |
| 30 | 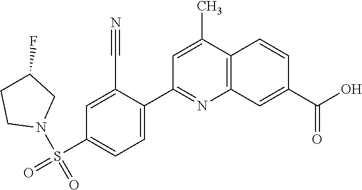 [SMILES: Cc1cc(-c2ccc(S(=O)(=O)N3CC[C@H](F)C3)cc2C#N)nc2cc(C(=O)O)ccc12] |
| 31 | 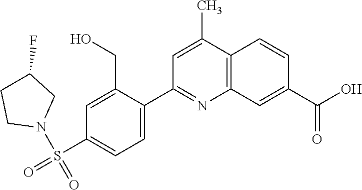 [SMILES: Cc1cc(-c2ccc(S(=O)(=O)N3CC[C@H](F)C3)cc2CO)nc2cc(C(=O)O)ccc12] |
| 32 | 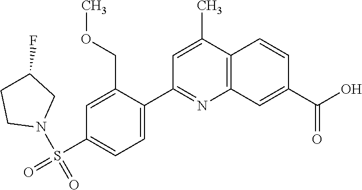 [SMILES: COCc1cc(S(=O)(=O)N2CC[C@H](F)C2)ccc1-c1cc(C)c2ccc(C(=O)O)cc2n1] |

TABLE 43

| Ex | Str |
|---|---|
| 33 | 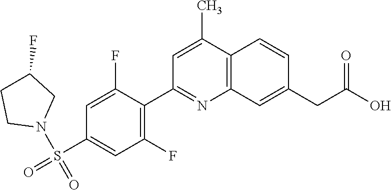 [SMILES: Cc1cc(-c2c(F)cc(S(=O)(=O)N3CC[C@H](F)C3)cc2F)nc2cc(CC(=O)O)ccc12] |
| 34 | 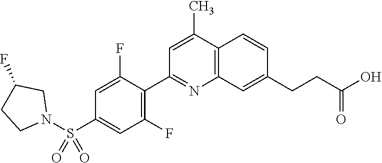 [SMILES: Cc1cc(-c2c(F)cc(S(=O)(=O)N3CC[C@H](F)C3)cc2F)nc2cc(CCC(=O)O)ccc12] |
| 35 | 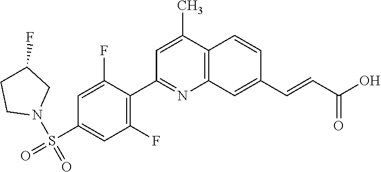 [SMILES: Cc1cc(-c2c(F)cc(S(=O)(=O)N3CC[C@H](F)C3)cc2F)nc2cc(/C=C/C(=O)O)ccc12] |
| 36 | 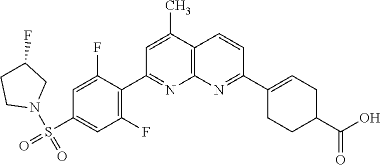 [SMILES: Cc1cc(-c2c(F)cc(S(=O)(=O)N3CC[C@H](F)C3)cc2F)nc2nc(C3=CCC(C(=O)O)CC3)ccc12] |

TABLE 44

| Ex | Str |
|---|---|
| 37 | 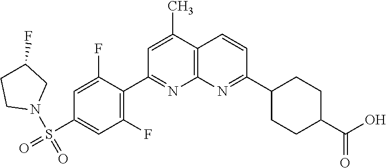 [SMILES: Cc1cc(-c2c(F)cc(S(=O)(=O)N3CC[C@H](F)C3)cc2F)nc2nc(C3CCC(C(=O)O)CC3)ccc12] |
| 38 | 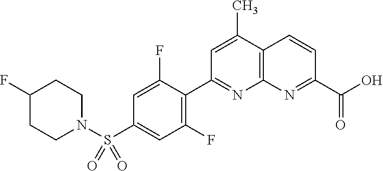 [SMILES: Cc1cc(-c2c(F)cc(S(=O)(=O)N3CCC(F)CC3)cc2F)nc2nc(C(=O)O)ccc12] |
| 39 | 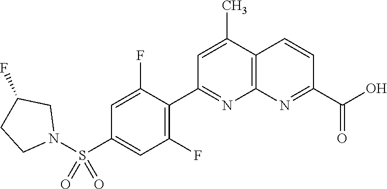 [SMILES: Cc1cc(-c2c(F)cc(S(=O)(=O)N3CC[C@H](F)C3)cc2F)nc2nc(C(=O)O)ccc12] |
| 40 | 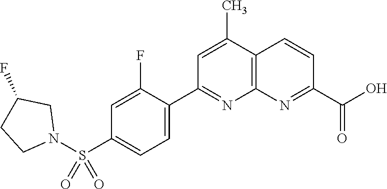 [SMILES: Cc1cc(-c2ccc(S(=O)(=O)N3CC[C@H](F)C3)cc2F)nc2nc(C(=O)O)ccc12] |

TABLE 45

| Ex | Str |
|---|---|
| 41 | 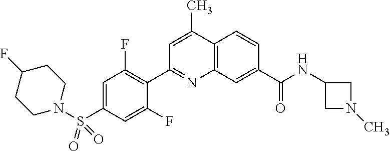 [SMILES: Cc1cc(-c2c(F)cc(S(=O)(=O)N3CCC(F)CC3)cc2F)nc2cc(C(=O)NC3CN(C)C3)ccc12] |
| 42 | 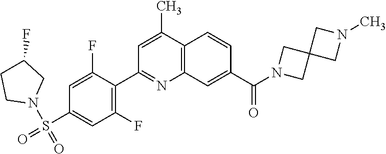 [SMILES: Cc1cc(-c2c(F)cc(S(=O)(=O)N3CC[C@H](F)C3)cc2F)nc2cc(C(=O)N3CC4(CN(C)C4)C3)ccc12] |
| 43 | 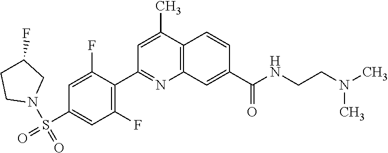 [SMILES: Cc1cc(-c2c(F)cc(S(=O)(=O)N3CC[C@H](F)C3)cc2F)nc2cc(C(=O)NCCN(C)C)ccc12] |
| 44 | 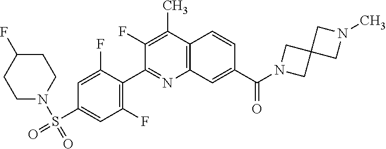 [SMILES: Cc1c(F)c(-c2c(F)cc(S(=O)(=O)N3CCC(F)CC3)cc2F)nc2cc(C(=O)N3CC4(CN(C)C4)C3)ccc12] |

TABLE 46

| Ex | Str |
|---|---|
| 45 | 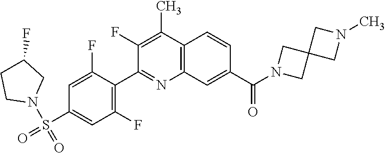 [SMILES: Cc1c(F)c(-c2c(F)cc(S(=O)(=O)N3CC[C@H](F)C3)cc2F)nc2cc(C(=O)N3CC4(CN(C)C4)C3)ccc12] |
| 46 | 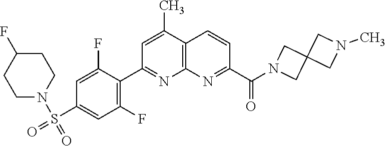 [SMILES: Cc1cc(-c2c(F)cc(S(=O)(=O)N3CCC(F)CC3)cc2F)nc2nc(C(=O)N3CC4(CN(C)C4)C3)ccc12] |
| 47 | 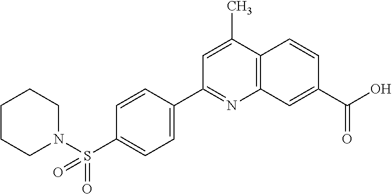 [SMILES: Cc1cc(-c2ccc(S(=O)(=O)N3CCCCC3)cc2)nc2cc(C(=O)O)ccc12] |
| 48 | 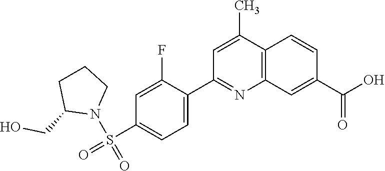 [SMILES: Cc1cc(-c2ccc(S(=O)(=O)N3CCC[C@H]3CO)cc2F)nc2cc(C(=O)O)ccc12] |

TABLE 47

| Ex | Str |
|---|---|
| 49 | 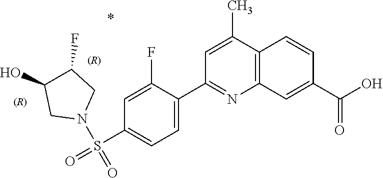 |
| 50 | 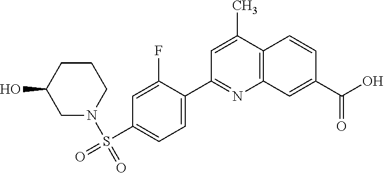 [SMILES: Cc1cc(-c2ccc(S(=O)(=O)N3CCC[C@H](O)C3)cc2F)nc2cc(C(=O)O)ccc12] |
| 51 | 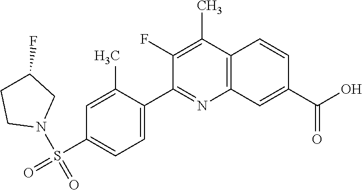 [SMILES: Cc1cc(S(=O)(=O)N2CC[C@H](F)C2)ccc1-c1nc2cc(C(=O)O)ccc2c(C)c1F] |
| 52 | 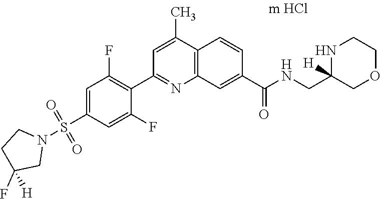 [SMILES: Cc1cc(-c2c(F)cc(S(=O)(=O)N3CC[C@H](F)C3)cc2F)nc2cc(C(=O)NC[C@H]3COCCN3)ccc12] |

TABLE 48

| Ex | Str |
|---|---|
| 53 | 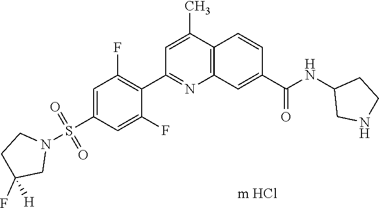 [SMILES: Cc1cc(-c2c(F)cc(S(=O)(=O)N3CC[C@H](F)C3)cc2F)nc2cc(C(=O)NC3CCNC3)ccc12] |
| 54 | 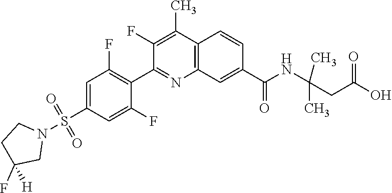 [SMILES: Cc1c(F)c(-c2c(F)cc(S(=O)(=O)N3CC[C@H](F)C3)cc2F)nc2cc(C(=O)NC(C)(C)CC(=O)O)ccc12] |
| 55 | 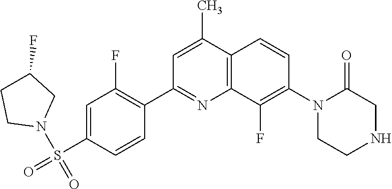 [SMILES: Cc1cc(-c2ccc(S(=O)(=O)N3CC[C@H](F)C3)cc2F)nc2c(F)c(N3CCNCC3=O)ccc12] |
| 56 | 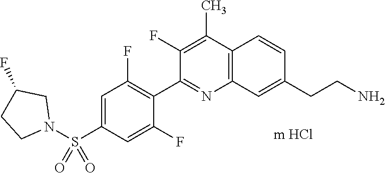 [SMILES: Cc1c(F)c(-c2c(F)cc(S(=O)(=O)N3CC[C@H](F)C3)cc2F)nc2cc(CCN)ccc12] |

TABLE 49

| Ex | Str |
|---|---|
| 57 | 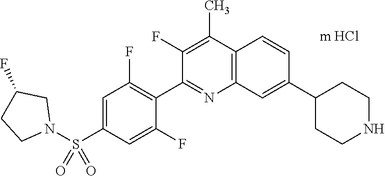 [SMILES: Cc1c(F)c(-c2c(F)cc(S(=O)(=O)N3CC[C@H](F)C3)cc2F)nc2cc(C3CCNCC3)ccc12] |
| 58 | 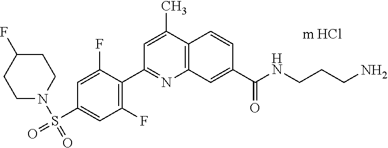 [SMILES: Cc1cc(-c2c(F)cc(S(=O)(=O)N3CCC(F)CC3)cc2F)nc2cc(C(=O)NCCCN)ccc12] |
| 59 | 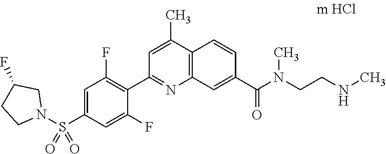 [SMILES: CNCCN(C)C(=O)c1ccc2c(C)cc(-c3c(F)cc(S(=O)(=O)N4CC[C@H](F)C4)cc3F)nc2c1] |
| 60 | 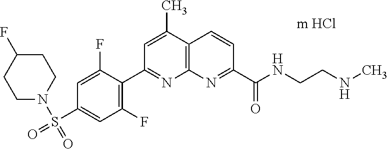 [SMILES: CNCCNC(=O)c1ccc2c(C)cc(-c3c(F)cc(S(=O)(=O)N4CCC(F)CC4)cc3F)nc2n1] |

TABLE 50

| Ex | Str |
|---|---|
| 61 | 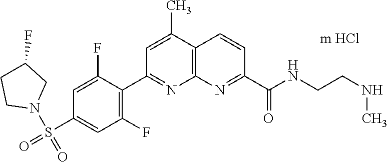 [SMILES: CNCCNC(=O)c1ccc2c(C)cc(-c3c(F)cc(S(=O)(=O)N4CC[C@H](F)C4)cc3F)nc2n1] |
| 62 | 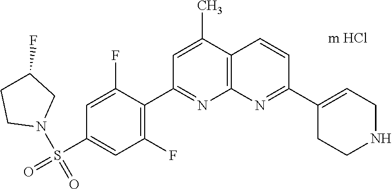 [SMILES: Cc1cc(-c2c(F)cc(S(=O)(=O)N3CC[C@H](F)C3)cc2F)nc2nc(C3=CCNCC3)ccc12] |
| 63 | 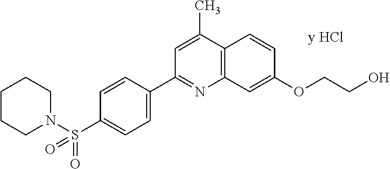 [SMILES: Cc1cc(-c2ccc(S(=O)(=O)N3CCCCC3)cc2)nc2cc(OCCO)ccc12] |
| 64 | 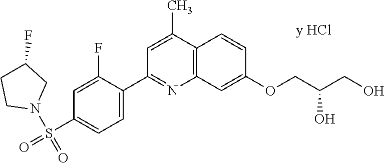 [SMILES: Cc1cc(-c2ccc(S(=O)(=O)N3CC[C@H](F)C3)cc2F)nc2cc(OC[C@@H](O)CO)ccc12] |

TABLE 51

| Ex | Str |
|---|---|
| 65 | 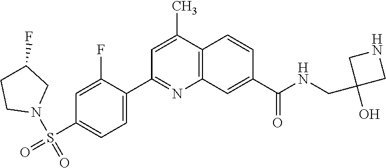 [SMILES: Cc1cc(-c2ccc(S(=O)(=O)N3CC[C@H](F)C3)cc2F)nc2cc(C(=O)NCC3(O)CNC3)ccc12] |
| 66 | 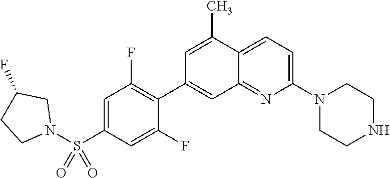 [SMILES: Cc1cc(-c2c(F)cc(S(=O)(=O)N3CC[C@H](F)C3)cc2F)cc2nc(N3CCNCC3)ccc12] |
| 67 | 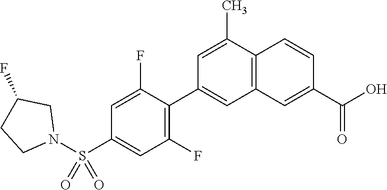 [SMILES: Cc1cc(-c2c(F)cc(S(=O)(=O)N3CC[C@H](F)C3)cc2F)cc2cc(C(=O)O)ccc12] |

TABLE 52

| Ex | Syn | DAT |
|---|---|---|
| 1 | E1 | ESI+; 451.3 |
| | | 1H NMR (500 MHz, DMSO-d₆) δ ppm 1.95-2.20 (2H, m), 2.77-2.82 (3H, |
| | | m), 3.15-3.72 (4H, m), 5.21-5.36 (1H, m), 7.73-7.82 (3H, m), 8.18 (1H, dd, |
| | | J = 8.7, 1.8 Hz), 8.25-8.32 (1H, m), 8.58 (1H, s), 13.15-13.64 (1H, m) |
| 2 | E2 | ESI+; 451.1 |
| | | 1H NMR (500 MHz, DMSO-d6) δ ppm 1.95-2.21 (2H, m), 2.73-2.76 (3H, |
| | | m), 3.19-3.41 (1H, m), 3.41-3.58 (2H, m), 3.60-3.71 (1H, m), 5.20-5.37 (1H, |
| | | m), 7.54 (1H, s), 7.71-7.81 (2H, m), 8.03 (1H, d, J = 8.6 Hz), 8.18 (1H, dd, |
| | | J = 8.6, 1.5 Hz), 8.41 (1H, d, J = 1.1 Hz) |
| 3 | E3 | ESI+; 559.4 |
| | | 1H NMR (500 MHz, DMSO-d6) δ ppm 1.78-1.89 (2H, m), 1.89-2.03 (2H, |
| | | m), 2.15 (3H, s), 2.76-2.81 (3H, m), 3.05-3.44 (8H, m), 4.17 (2H, s), 4.46 (2H, |
| | | s), 4.72-4.90 (1H, m), 7.70-7.77 (3H, m), 7.91 (1H, dd, J = 8.7, 1.7 Hz), 8.23 |
| | | (1H, d, J = 1.4 Hz ), 8.26 (1H, d, J = 8.7 Hz ) |
| 4 | E4 | ESI+; 519.3 |
| | | 1H NMR (500 MHz, DMSO-d6) δ ppm 1.74-2.04 (4H, m), 2.04-2.30 (2H, |
| | | m), 2.74-2.83 (3H, m), 3.06-3.37 (4H, m), 3.68-3.81 (2H, m), 3.91-4.03 (1H, |
| | | m), 4.19-4.34 (1H, m), 4.45-4.54 (1H, m), 4.73-4.88 (1H, m), 7.67-7.80 (3H, |
| | | m), 7.91 (1H, dd, J = 8.7, 1.7 Hz), 8.21 (1H, d, J = 1.4 Hz), 8.26 (1H, d, J = |
| | | 8.7 Hz) |
| 5 | E5 | ESI+; 447.2 |
| 6 | E6 | ESI+; 482.1 |
| 7 | E7 | ESI+; 451.2 |
| 8 | E8 | ESI+; 463.1 |
| 9 | E9 | ESI+; 536.2 |
| 10 | E10 | ESI+; 507.2 |

TABLE 53

| Ex | Syn | DAT |
|---|---|---|
| 11 | E11 | ESI+; 566.2 |
| 12 | E12 | ESI+; 537.2 |
| 13 | E13 | ESI+; 448.2 |
| 14 | E14 | ESI+; 545.2 |
| 15 | E15 | ESI+; 525.2 |
| 16 | E16 | ESI+; 496.2 |
| 17 | E17 | ESI+; 505.3 |
| 18 | E18 | ESI+; 483.1 |
| | | 1H NMR (500 MHz, DMSO-d6) δ ppm 1.76-1.89 (2H, m), 1.89-2.05 (2H, |
| | | m), 2.73 (3H, d, J = 2.1 Hz), 3.09-3.52 (4H, m), 4.71-4.91 (1H, m), 7.77-7.84 |
| | | (2H, m), 8.23 (1H, dd, J = 8.8, 1.6 Hz), 8.34 (1H, d, J = 8.9 Hz), 8.62 (1H, d, |
| | | J = 1.4 Hz), 13.48 (1H, brs) |
| 19 | E19 | ESI+; 465.2 |
| | | 1H NMR (500 MHz, DMSO-d6) δ ppm 1.77-1.89 (2H, m), 1.89-2.04 (2H, |
| | | m), 2.75-2.86 (3H, m), 3.02-3.54 (4H, m), 4.71-4.91 (1H, m), 7.70-7.76 (2H, |
| | | m), 7.78 (1H, s), 8.18 (1H, dd, J = 8.7, 1.7 Hz), 8.28-8.34 (1H, m), 8.59 (1H, d, |
| | | J = 1.5 Hz), 12.83-13.95 (1H, m) |
| 20 | E20 | ESI+; 469.2 |
| | | 1H NMR (500 MHz, DMSO-d6) δ ppm 1.95-2.20 (2H, m), 2.73 (3H, d, J = 2.1 |
| | | Hz), 3.19-3.44 (1H, m), 3.44-3.59 (2H, m), 3.62-3.76 (1H, m), 5.19-5.38 (1H, |
| | | m), 7.86 (2H, d, J = 6.6 Hz), 8.23 (1H, dd, J = 8.7, 1.5 Hz), 8.34 (1H, d, J = 8.7 |
| | | Hz), 8.61 (1H, d, J = 1.4 Hz), 13.47 (1H, brs) |
| 21 | E1 | ESI+; 433.3 |
| 22 | E1 | ESI+; 451.2 |
| 23 | E1 | ESI+; 451.2 |
| 24 | E1 | ESI+; 465.2 |

TABLE 54

| Ex | Syn | DAT |
|---|---|---|
| 25 | E1 | ESI+; 477.2 |
| 26 | E1 | ESI+; 479.2 |
| 27 | E1 | ESI+; 451.2 |
| 28 | E1 | ESI+; 463.2 |
| 29 | E1 | ESI+; 463.2 |
| 30 | E1 | ESI+; 440.3 |
| 31 | E1 | ESI+; 445.2 |
| 32 | E1 | ESI+; 459.2 |
| 33 | E1 | ESI+; 465.2 |
| 34 | E1 | ESI+; 479.2 |
| 35 | E1 | ESI+; 477.2 |
| 36 | E1 | ESI+; 532.4 |
| 37 | E1 | ESI+; 534.2 |
| 38 | E1 | ESI+; 466.2 |
| 39 | E1 | ESI+; 452.2 |
| 40 | E5 | ESI+; 433.2 |
| 41 | E3 | ESI+; 533.4 |
| 42 | E3 | ESI+; 545.3 |
| 43 | E3 | ESI+; 521.3 |
| 44 | E3 | ESI+; 577.3 |
| 45 | E3 | ESI+; 563.3 |
| 46 | E3 | ESI+; 560.2 |
| 47 | E5 | ESI+; 411.2 |
| 48 | E5 | ESI+; 445.2 |
| 49 | E5 | ESI+; 449.2 |
| 50 | E5 | ESI+; 445.2 |

TABLE 55

| Ex | Syn | DAT |
|---|---|---|
| 51 | E7 | ESI+; 447.2 |
| 52 | E10 | ESI+; 549.2 |
| 53 | E10 | ESI+; 519.2 |
| 54 | E11 | ESI+; 568.3 |
| 55 | E13 | ESI+; 505.3 |
| 56 | E13 | ESI+; 468.2 |
| 57 | E13 | ESI+; 508.2 |
| 58 | E13 | ESI+; 521.2 |
| 59 | E13 | ESI+; 521.2 |
| 60 | E13 | ESI+; 522.3 |
| 61 | E13 | ESI+; 508.2 |
| 62 | E13 | ESI+; 489.2 |
| 63 | E13 | ESI+; 427.3 |
| 64 | E13 | ESI+; 479.3 |
| 65 | E14 | ESI+; 517.3 |
| 66 | E14 | ESI+; 491.2 |
| 67 | E1 | ESI+; 450.3 |

Apart from the present invention, compound S1 can be produced by using the methods described in the above-mentioned production methods and examples, synthetic methods of sulfonimide compounds (refer to supporting information of Organic. Letters, 22: pp 2702-2706 (2020)), methods that are obvious to a person skilled in the art, or modifications thereof and can be expected to have an inducing action of acetylcholine receptor clustering and to be used for preventing and/or treating neuromuscular diseases.

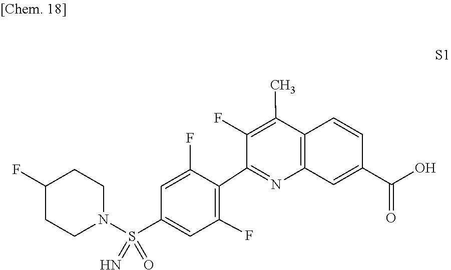 [SMILES: Cc1c(F)c(-c2c(F)cc(S(=N)(=O)N3CCC(F)CC3)cc2F)nc2cc(C(=O)O)ccc12]

While the invention has been described in detail and with reference to specific embodiments thereof, it will be apparent to one skill in the art that various changes and modifications can be made therein without departing from the spirit and scope thereof. All references cited herein are incorporated in their entirety. This application is based on International application No. PCT/CN2021/141500 filed on Dec. 27, 2021, the entire contents of which are incorporated hereinto by reference.

### INDUSTRIAL APPLICABILITY

The compound of formula (I) or a salt thereof has an acetylcholine receptor clustering-inducing action and can be useful as an agent for preventing and/or treating neuromuscular diseases.

1. A compound of formula (I) or a salt thereof: 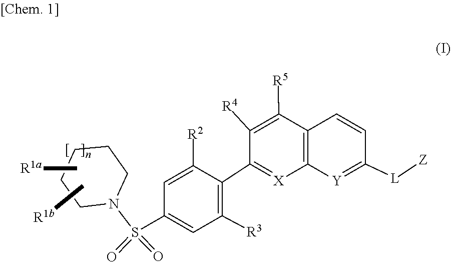
wherein,
R^{1a} and R^{1b} each are the same or different, and are H, optionally substituted C₁₋₆ alkyl, halogen, hydroxy, or —O-(optionally substituted C₁₋₆ alkyl), and when R^{1a} and R^{1b} are attached to the same carbon atom, R^{1a} and R^{1b} may be linked to each other to form C₃₋₈ cycloalkyl group together with the carbon atom to which R^{1a} and R^{1b} are attached,
R² is H, optionally substituted C₁₋₆ alkyl, halogen, cyano, or —O-(optionally substituted C₁₋₆ alkyl),
R³ is H or halogen,
R⁴ is H, methyl, or halogen,
R⁵ is methyl, ethyl, or fluoromethyl,
X is N or CR^{X} and Y is N or CR^{Y},
R^{X} is H or halogen,
R^{Y} is H or halogen,
L is a bond, C₁₋₆ alkylene, —O—(C₁₋₆ alkylene), C₂₋₆ alkenylene, C₃₋₈ cycloalkylene, or C₄₋₈ cycloalkenylene,
Z is —COOH or —CONR^{Z1}R^{Z2}, or
L and Z may together form optionally substituted C₁₋₆ alkyl, —O-(optionally substituted C₁₋₆ alkyl), or optionally substituted 3- to 8-membered heterocyclic ring group comprising 1 to 2 nitrogen atoms,
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₁₋₆ alkyl or C₃₋₈ cycloalkyl,
R^{Z2} is H or C₁₋₆ alkyl, or
R^{Z1} and R^{Z2} may be linked to each other to form optionally substituted 3- to 8-membered heterocyclic ring group comprising 1 to 2 nitrogen atoms together with the nitrogen atom to which R^{Z1} and R^{Z2} are attached, and,
n is 0 or 1.

2. The compound or a salt thereof according to claim 1, wherein
R^{1a} and R^{1b} each are the same or different, and are H, methyl, hydroxymethyl, fluoro, hydroxy, or methoxy, and when R^{1a} and R^{1b} are attached to the same carbon atom, R^{1a} and R^{1b} may be linked to each other to form cyclopropyl group together with the carbon atom to which R^{1a} and R^{1b} are attached,
R² is H, methyl, hydroxymethyl, methoxymethyl, fluoro, cyano, or methoxy,
R³ is H or fluoro,
R⁴ is H or fluoro,
R⁵ is methyl,
X is N or CR^{X} and Y is N or CR^{Y},
R^{X} is H or fluoro,
R^{Y} is H or fluoro,
L is a bond, methylene, ethylene, —OCH₂—, ethenylene, cyclohexanediyl, or cyclohexenediyl,
Z is —COOH or —CONR^{Z1}R^{Z2}, or
L and Z may together form C₁₋₃ alkyl optionally substituted with a substituent selected from the group consisting of —NH₂ and —NH-methyl, —O—(C₁₋₃ alkyl optionally substituted with 1 to 2 substituents selected from the group consisting of hydroxy and —NH₂), or 3- to 8-membered heterocyclic ring group comprising 1 to 2 nitrogen atoms optionally substituted with a substituent selected from the group consisting of methyl and oxo,
R^{Z1} is C₁₋₃ alkyl optionally substituted with 1 to 2 substituents selected from the group consisting of —NH₂, —NH-methyl, —N(methyl)₂ and morpholinyl, 3- to 8-membered heterocyclic ring group comprising 1 to 2 nitrogen atoms optionally substituted with a methyl, or —SO₂—R^{Z3},
or R^{Z1} is the substituent selected from the group consisting of the following formula (i), formula (ii) and formula (iii), 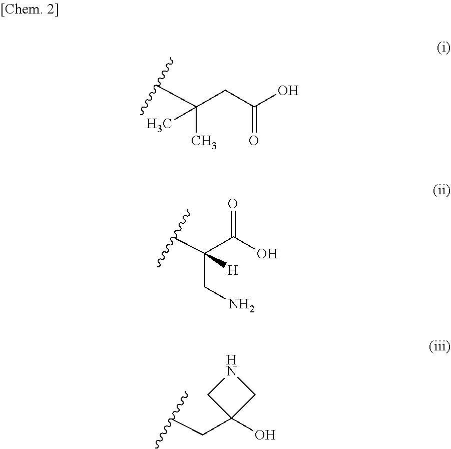
R^{Z3} is a cyclopropyl,
R^{Z2} is H or methyl, or
R^{Z1} and R^{Z2} may be linked to each other to form structure(s) of the following formula (iv), or formula (v) together with the nitrogen atom to which R^{Z1} and R^{Z2} are attached, and 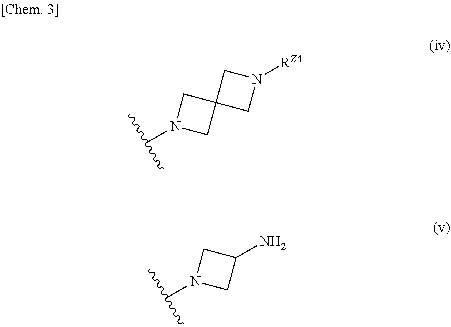
R^{Z4} is H or methyl.

3. A compound of formula (I) or a salt thereof: 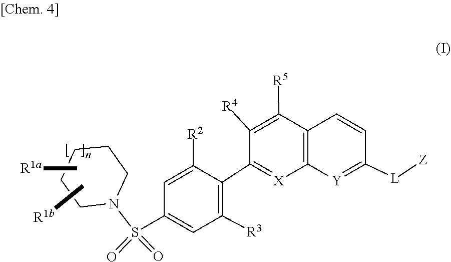
wherein,
R^{1a} and R^{1b} each are the same or different, and are H, optionally substituted C₁₋₆ alkyl, halogen, hydroxy, or —O-(optionally substituted C₁₋₆ alkyl), and when R^{1a} and R^{1b} are attached to the same carbon atom, R^{1a} and R^{1b} may be linked to each other to form C₃₋₈ cycloalkyl group together with the carbon atom to which R^{1a} and R^{1b} are attached,
R² is H, optionally substituted C₁₋₆ alkyl, halogen, cyano, or —O-(optionally substituted C₁₋₆ alkyl),
R³ is H or halogen,
R⁴ is H, methyl, or halogen,
R⁵ is methyl, ethyl, or fluoromethyl,
X is N or CR^{X} and Y is N or CR^{Y}, provided that X and Y are not CR^{X} and CR^{Y} at the same time,
R^{X} is H or halogen,
R^{Y} is H or halogen,
L is a bond, C₁₋₆ alkylene, —O—(C₁₋₆ alkylene), C₂₋₆ alkenylene, C₃₋₈ cycloalkylene, or C₄₋₈ cycloalkenylene,
Z is —COOH or —CONR^{Z1}R^{Z2}, or
L and Z may together form optionally substituted C₁₋₆ alkyl, —O-(optionally substituted C₁₋₆ alkyl), or optionally substituted 3- to 8-membered heterocyclic ring group comprising 1 to 2 nitrogen atoms,
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₁₋₆ alkyl or C₃₋₈ Cycloalkyl,
R^{Z2} is H or C₁₋₆ alkyl, or
R^{Z1} and R⁷² may be linked to each other to form optionally substituted 3- to 8-membered heterocyclic ring group comprising 1 to 2 nitrogen atoms together with the nitrogen atom to which R^{Z1} and R^{Z2} are attached, and,
n is 0 or 1.

4. The compound or a salt thereof according to claim 3, wherein
R⁴ is H, or halogen,
R⁵ is methyl,
L is a bond, C₁₋₆ alkylene, —O—(C₁₋₆ alkylene), C₂₋₆ alkenylene, C₃₋₈ cycloalkylene, or C₄₋₈ cycloalkenylene,
Z is —COOH or —CONR^{Z1}R^{Z2}, or
L and Z may together form C₁₋₆ alkyl optionally substituted with the 1 to 2 substituents selected from the group consisting of hydroxy and —NHR^{LZ}, —O—(C₁₋₆ alkyl optionally substituted with 1 to 2 substituents selected from the group consisting of hydroxy and —NHR^{LZ}), or 3- to 8-membered heterocyclic ring group comprising 1 to 2 nitrogen atoms optionally substituted with the 1 to 2 substituents selected from the group consisting of C₁₋₆ alkyl and oxo and
R^{LZ} is H or C₁₋₆ alkyl,
R^{Z1} is optionally substituted C₁₋₆ alkyl, optionally substituted 3- to 8-membered heterocyclic ring group comprising 1 to 2 nitrogen atoms, or —SO₂—R^{Z3},
R^{Z3} is C₃₋₈ cycloalkyl,
R^{Z2} is H or C₁₋₆ alkyl, or
R^{Z1} and R^{Z2} may be linked to each other to form optionally substituted 3- to 8-membered heterocyclic ring group comprising 1 to 2 nitrogen atoms together with the nitrogen atom to which R^{Z1} and R^{Z2} are attached.

5. The compound or a salt thereof according to claim 4, wherein
R^{1a} and R^{1b} each are the same or different, and are H, methyl, hydroxymethyl, fluoro, hydroxy, or methoxy, and when R^{1a} and R^{1b} are attached to the same carbon atom, R^{1a} and R^{1b} may be linked to each other to form cyclopropyl group together with the carbon atom to which R^{1a} and R^{1b} are attached,
R² is H, methyl, hydroxymethyl, methoxymethyl, fluoro, cyano, or methoxy,
R³ is H or fluoro,
R⁴ is H or fluoro,
R^{X} is H or fluoro,
R^{Y} is H or fluoro,
L is a bond, methylene, ethylene, —OCH₂—, ethenylene, cyclohexanediyl, or cyclohexenediyl,
Z is —COOH or —CONR^{Z1}R^{Z2}, or
L and Z may together form C₁₋₃ alkyl optionally substituted with a substituent selected from the group consisting of —NH₂ and —NH-methyl, —O—(C₁₋₃ alkyl optionally substituted with 1 to 2 substituents selected from the group consisting of hydroxy and —NH₂), or 3- to 8-membered heterocyclic ring group comprising 1 to 2 nitrogen atoms optionally substituted with a substituent selected from the group consisting of methyl and oxo,
R^{Z1} is C₁₋₃ alkyl optionally substituted with 1 to 2 substituents selected from the group consisting of —NH₂, —NH-methyl, —N(methyl)₂ and morpholinyl, 3- to 8-membered heterocyclic ring group comprising 1 to 2 nitrogen atoms optionally substituted with a methyl, or —SO₂—R^{Z3},
or R^{Z1} is the substituent selected from the group consisting of the following formula (i), formula (ii) and formula (iii), 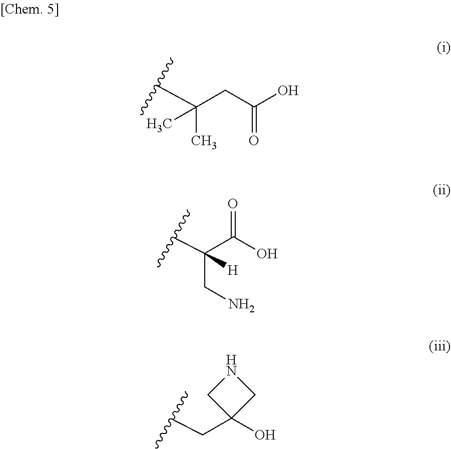
R^{Z3} is a cyclopropyl,
R^{Z2} is H or methyl, or
R^{Z1} and R^{Z2} may be linked to each other to form structure(s) of the following formula (iv), or formula (v) together with the nitrogen atom to which R^{Z1} and R^{Z2} are attached, and 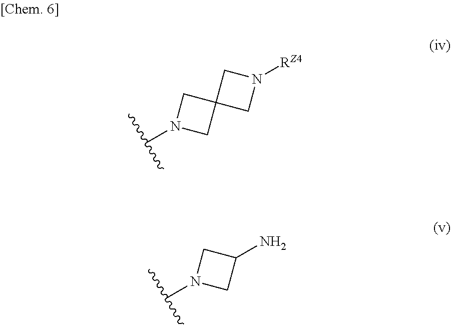
R^{Z4} is H or methyl.

6. The compound or a salt thereof according to claim 5, wherein R^{1a} and R^{1b} each are the same or different and, are H or fluoro,
R² is fluoro,
R³ is fluoro,
X is N and Y is CR^{Y},
R^{Y} is H,
L is a bond,
Z is —COOH or —CONR^{Z1}R^{Z2},
R^{Z1} and R^{Z2} are linked to each other to form structure(s) of the following formula (vi) or formula (v) together with the nitrogen atom to which R^{Z1} and R^{Z2} are attached.

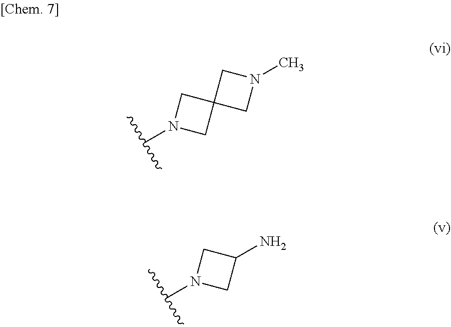

7. The compound or a salt thereof according to claim 1, wherein the compound is selected from the group consisting of
2-{2,6-Difluoro-4-[(3S)-3-fluoropyrrolidine-1-sulfonyl]phenyl}-4-meth ylquinoline-7-carboxylic acid,
Sodium 2-{2,6-difluoro-4-[(3S)-3-fluoropyrrolidine-1-sulfonyl]phenyl}-4-meth ylquinoline-7-carboxylate,
{2-[2,6-Difluoro-4-(4-fluoropiperidine-1-sulfonyl)phenyl]-4-methylquinolin-7-yl}(6-methyl-2,6-diazaspiro[3.3]heptan-2-yl) methanone,
(3-Aminoazetidin-1-yl) {2-[2,6-difluoro-4-(4-fluoropiperidine-1-sulfonyl)phenyl]-4-methylquinolin-7-yl}methanone,
2-[2,6-Difluoro-4-(4-fluoropiperidine-1-sulfonyl)phenyl]-3-fluoro-4-methylquinoline-7-carboxylic acid,
2-[2,6-Difluoro-4-(4-fluoropiperidine-1-sulfonyl)phenyl]-4-methylquinoline-7-carboxylic acid, and
2-{2,6-Difluoro-4-[(3S)-3-fluoropyrrolidine-1-sulfonyl]phenyl}-3-fluoro-4-methylquinoline-7-carboxylic acid.

8. A pharmaceutical composition comprising the compound or a salt thereof according to claim 1 and a pharmaceutically acceptable excipient.

9. The pharmaceutical composition according to claim 8, which is a pharmaceutical composition for preventing and/or treating neuromuscular diseases.

10. Use of the compound or a salt thereof according to claim 1 for the manufacture of a pharmaceutical composition for preventing and/or treating neuromuscular diseases.

11. Use of the compound or a salt thereof according to claim 1 for preventing and/or treating neuromuscular diseases.

12. The compound or a salt thereof according to claim 1 for use in preventing and/or treating of neuromuscular diseases.

13. A method for preventing and/or treating neuromuscular diseases, comprising administering an effective amount of the compound or a salt thereof according to claim 1 to a subject.
